SEQUENCE LISTING (190 sequences)

SEQ ID NO: 1 - RNA, 23 nt, Homo sapiens
ugugcaaaucuaugcaaaacuga

SEQ ID NO: 2 - RNA, 22 nt, Homo sapiens
aguuuugcauaguugcacuaca

SEQ ID NO: 3 - RNA, 23 nt, Homo sapiens
ugugcaaauccaugcaaaacuga

SEQ ID NO: 4 - RNA, 23 nt, Homo sapiens
aguuuugcagguuugcauccagc

SEQ ID NO: 5 - RNA, 22 nt, Homo sapiens
aguuuugcagguuugcauuuca

SEQ ID NO: 6 - RNA, 23 nt, Mus sp.
ugugcaaaucuaugcaaaacuga

SEQ ID NO: 7 - RNA, 22 nt, Mus sp.
uaguuuugcauaguugcacuac

SEQ ID NO: 8 - RNA, 23 nt, Mus sp.
ugugcaaauccaugcaaaacuga

SEQ ID NO: 9 - RNA, 23 nt, Mus sp.
aguuuugcagguuugcauccagc

SEQ ID NO: 10 - RNA, 24 nt, Mus sp.
aguuuugcagauuugcaguucagc

SEQ ID NO: 11 - DNA, 16 bp, Artificial Sequence
  19b_LNA_DNA_PS_16 miR-19 inhibitor
  misc_feature (1)..(16): May be joined by phosphorothioate bonds
  modified_base (1)..(1): May be a locked nucleic acid thymidine
  modified_base (5)..(5): May be a locked nucleic acid adenosine
  modified_base (6)..(6): May be a locked nucleic acid thymidine
  modified_base (8)..(8): May be a locked nucleic acid guanosine
  modified_base (10)..(11): May be a locked nucleic acid thymidine
  modified_base (13)..(13): May be a locked nucleic acid guanosine
  modified_base (15)..(15): May be a locked nucleic acid adenosine
  modified_base (16)..(16): May be a locked nucleic acid cytidine
ttgcatggatttgcac

SEQ ID NO: 12 - DNA, 16 bp, Artificial Sequence
  19a_LNA_DNA_PS_16 miR-19 inhibitor
  misc_feature (1)..(16): May be joined by phosphorothioate bonds
  modified_base (1)..(1): May be a locked nucleic acid thymidine
  modified_base (5)..(5): May be a locked nucleic acid adenosine
  modified_base (6)..(6): May be a locked nucleic acid thymidine
  modified_base (8)..(8): May be a locked nucleic acid guanosine
  modified_base (10)..(11): May be a locked nucleic acid thymidine
  modified_base (13)..(13): May be a locked nucleic acid guanosine
  modified_base (15)..(15): May be a locked nucleic acid adenosine
  modified_base (16)..(16): May be a locked nucleic acid cytidine
ttgcatagatttgcac

SEQ ID NO: 13 - RNA, 22 nt, Homo sapiens
uauugcacuugucccggccugu

SEQ ID NO: 14 - RNA, 23 nt, Homo sapiens
agguugggaucgguugcaaugcu

SEQ ID NO: 15 - RNA, 22 nt, Homo sapiens
ggguggggauuuguugcauuac

SEQ ID NO: 16 - RNA, 21 nt, Mus sp.
uauugcacuugucccggccug

SEQ ID NO: 17 - RNA, 23 nt, Mus sp.
agguugggauuugucgcaaugcu

SEQ ID NO: 18 - RNA, 22 nt, Mus sp.
agguggggauugguggcauuac

SEQ ID NO: 19 - RNA, 21 nt, Rattus sp.
uauugcacuugucccggccug

SEQ ID NO: 20 - RNA, 23 nt, Rattus sp.
agguugggauuugucgcaaugcu

SEQ ID NO: 21 - RNA, 22 nt, Rattus sp.
agguggggauuagugccauuac

SEQ ID NO: 22 - DNA, 16 bp, Artificial Sequence
  92a_LNA_16_PS miR-92 inhibitor
  misc_feature (1)..(16): May be joined by phosphorothioate bonds
  modified_base (1)..(1): May be a locked nucleic acid cytidine
  modified_base (3)..(3): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 23 - DNA, 16 bp, Artificial Sequence
  92a_LNA_16_1 miR-92 inhibitor
  misc_feature (1)..(16): May be joined by phosphorothioate bonds
  modified_base (1)..(1): May be a locked nucleic acid cytidine
  modified_base (5)..(5): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (15)..(15): May be a locked nucleic acid adenosine
  modified_base (16)..(16): May be a locked nucleic acid thymidine
ccgggacaagtgcaat

SEQ ID NO: 24 - DNA, 16 bp, Artificial Sequence
  92a_LNA_16_4 miR-92 inhibitor
  misc_feature (1)..(16): May be joined by phosphorothioate bonds
  modified_base (1)..(1): May be a locked nucleic acid cytidine
  modified_base (3)..(3): May be a locked nucleic acid guanosine
  modified_base (6)..(6): May be a locked nucleic acid adenosine
  modified_base (9)..(9): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 25 - DNA, 8 bp, Artificial Sequence
  92a_Tiny_LNA miR-92 inhibitor
  misc_feature (1)..(8): May be joined by phosphorothioate bonds
  modified_base (1)..(1): May be a locked nucleic acid adenosine
  modified_base (2)..(2): May be a locked nucleic acid guanosine
  modified_base (3)..(3): May be a locked nucleic acid thymidine
  modified_base (4)..(4): May be a locked nucleic acid guanosine
  modified_base (5)..(5): May be a locked nucleic acid cytidine
  modified_base (6)..(7): May be a locked nucleic acid adenosine
  modified_base (8)..(8): May be a locked nucleic acid thymidine
agtgcaat

SEQ ID NO: 26 - DNA, 16 bp, Artificial Sequence
  92a_LNA_16_2 miR-92 inhibitor
  misc_feature (1)..(16): May be joined by phosphorothioate bonds
  modified_base (1)..(1): May be a locked nucleic acid cytidine
  modified_base (3)..(4): May be a locked nucleic acid guanosine
  modified_base (7)..(7): May be a locked nucleic acid cytidine
  modified_base (8)..(8): May be a locked nucleic acid adenosine
  modified_base (10)..(10): May be a locked nucleic acid guanosine
  modified_base (12)..(12): May be a locked nucleic acid guanosine
  modified_base (14)..(14): May be a locked nucleic acid adenosine
  modified_base (16)..(16): May be a locked nucleic acid thymidine
ccgggacaagtgcaat

SEQ ID NO: 27 - DNA, 16 bp, Artificial Sequence
  92a_LNA_16_3 miR-92 inhibitor
  misc_feature (1)..(16): May be joined by phosphorothioate bonds
  modified_base (1)..(1): May be a locked nucleic acid cytidine
  modified_base (4)..(5): May be a locked nucleic acid guanosine
  modified_base (7)..(7): May be a locked nucleic acid cytidine
  modified_base (9)..(9): May be a locked nucleic acid adenosine
  modified_base (10)..(10): May be a locked nucleic acid guanosine
  modified_base (12)..(12): May be a locked nucleic acid guanosine
  modified_base (14)..(14): May be a locked nucleic acid adenosine
  modified_base (16)..(16): May be a locked nucleic acid thymidine
ccgggacaagtgcaat

SEQ ID NO: 28 - DNA, 16 bp, Artificial Sequence
  92a_LNA_16_5 miR-92 inhibitor
  misc_feature (1)..(16): May be joined by phosphorothioate bonds
  modified_base (1)..(1): May be a locked nucleic acid cytidine
  modified_base (3)..(3): May be a locked nucleic acid guanosine
  modified_base (5)..(5): May be a locked nucleic acid guanosine
  modified_base (7)..(7): May be a locked nucleic acid cytidine
  modified_base (9)..(9): May be a locked nucleic acid adenosine
  modified_base (11)..(11): May be a locked nucleic acid thymidine
  modified_base (13)..(13): May be a locked nucleic acid cytidine
  modified_base (15)..(15): May be a locked nucleic acid adenosine
  modified_base (16)..(16): May be a locked nucleic acid thymidine
ccgggacaagtgcaat

SEQ ID NO: 29 - DNA, 16 bp, Artificial Sequence
  92a_LNA_16_6 miR-92 inhibitor
  misc_feature (1)..(16): May be joined by phosphorothioate bonds
  modified_base (1)..(2): May be a locked nucleic acid cytidine
  modified_base (4)..(4): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (12)..(12): May be a locked nucleic acid guanosine
  modified_base (14)..(14): May be a locked nucleic acid adenosine
  modified_base (16)..(16): May be a locked nucleic acid thymidine
ccgggacaagtgcaat

SEQ ID NO: 30 - DNA, 16 bp, Artificial Sequence
  92a_LNA_16_7 miR-92 inhibitor
  misc_feature (1)..(16): May be joined by phosphorothioate bonds
  modified_base (1)..(1): May be a locked nucleic acid cytidine
  modified_base (5)..(5): May be a locked nucleic acid guanosine
  modified_base (8)..(8): May be a locked nucleic acid adenosine
  modified_base (11)..(11): May be a locked nucleic acid thymidine
  modified_base (12)..(12): May be a locked nucleic acid guanosine
  modified_base (13)..(13): May be a locked nucleic acid cytidine
  modified_base (14)..(15): May be a locked nucleic acid adenosine
  modified_base (16)..(16): May be a locked nucleic acid thymidine
ccgggacaagtgcaat

SEQ ID NO: 31 - DNA, 16 bp, Artificial Sequence
  92a_LNA_16_8 miR-92 inhibitor
  misc_feature (1)..(16): May be joined by phosphorothioate bonds
  modified_base (1)..(1): May be a locked nucleic acid cytidine
  modified_base (4)..(4): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 32 - DNA, 16 bp, Artificial Sequence
  92a_LNA_16_9 miR-92 inhibitor
  misc_feature (1)..(16): May be joined by phosphorothioate bonds
  modified_base (1)..(1): May be a locked nucleic acid cytidine
  modified_base (3)..(3): May be a locked nucleic acid guanosine
  modified_base (5)..(5): May be a locked nucleic acid guanosine
  modified_base (8)..(8): May be a locked nucleic acid adenosine
  modified_base (10)..(10): May be a locked nucleic acid guanosine
  modified_base (12)..(12): May be a locked nucleic acid guanosine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 33 - DNA, 16 bp, Artificial Sequence
  92a_LNA_16_10 miR-92 inhibitor
  misc_feature (1)..(16): May be joined by phosphorothioate bonds
  modified_base (1)..(1): May be a locked nucleic acid cytidine
  modified_base (3)..(3): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (14)..(15): May be a locked nucleic acid adenosine
  modified_base (16)..(16): May be a locked nucleic acid thymidine
ccgggacaagtgcaat

SEQ ID NO: 34 - DNA, 16 bp, Artificial Sequence
  92a_LNA_16_11 miR-92 inhibitor
  misc_feature (1)..(16): May be joined by phosphorothioate bonds
  modified_base (1)..(2): May be a locked nucleic acid cytidine
  modified_base (3)..(3): May be a locked nucleic acid guanosine
  modified_base (6)..(6): May be a locked nucleic acid adenosine
  modified_base (8)..(8): May be a locked nucleic acid adenosine
  modified_base (11)..(11): May be a locked nucleic acid thymidine
  modified_base (14)..(15): May be a locked nucleic acid adenosine
  modified_base (16)..(16): May be a locked nucleic acid thymidine
ccgggacaagtgcaat

SEQ ID NO: 35 - DNA, 16 bp, Artificial Sequence
  92a_LNA_16_12 miR-92 inhibitor
  misc_feature (1)..(16): May be joined by phosphorothioate bonds
  modified_base (1)..(2): May be a locked nucleic acid cytidine
  modified_base (3)..(3): May be a locked nucleic acid guanosine
  modified_base (5)..(5): May be a locked nucleic acid guanosine
  modified_base (8)..(8): May be a locked nucleic acid adenosine
  modified_base (11)..(11): May be a locked nucleic acid thymidine
  modified_base (14)..(15): May be a locked nucleic acid adenosine
  modified_base (16)..(16): May be a locked nucleic acid thymidine
ccgggacaagtgcaat

SEQ ID NO: 36 - DNA, 16 bp, Artificial Sequence
  92a_LNA_16_13 miR-92 inhibitor
  misc_feature (1)..(16): May be joined by phosphorothioate bonds
  modified_base (1)..(2): May be a locked nucleic acid cytidine
  modified_base (3)..(3): May be a locked nucleic acid guanosine
  modified_base (6)..(6): May be a locked nucleic acid adenosine
  modified_base (8)..(8): May be a locked nucleic acid adenosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 37 - DNA, 16 bp, Artificial Sequence
  92a_LNA_16_14 miR-92 inhibitor
  misc_feature (1)..(16): May be joined by phosphorothioate bonds
  modified_base (1)..(1): May be a locked nucleic acid cytidine
  modified_base (3)..(3): May be a locked nucleic acid guanosine
  modified_base (5)..(5): May be a locked nucleic acid guanosine
  modified_base (7)..(7): May be a locked nucleic acid cytidine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (15)..(15): May be a locked nucleic acid adenosine
  modified_base (16)..(16): May be a locked nucleic acid thymidine
ccgggacaagtgcaat

SEQ ID NO: 38 - DNA, 16 bp, Artificial Sequence
  92a_LNA_16_15 miR-92 inhibitor
  misc_feature (1)..(16): May be joined by phosphorothioate bonds
  modified_base (1)..(2): May be a locked nucleic acid cytidine
  modified_base (5)..(5): May be a locked nucleic acid guanosine
  modified_base (7)..(7): May be a locked nucleic acid cytidine
  modified_base (9)..(9): May be a locked nucleic acid adenosine
  modified_base (10)..(10): May be a locked nucleic acid guanosine
  modified_base (12)..(12): May be a locked nucleic acid guanosine
  modified_base (13)..(13): May be a locked nucleic acid cytidine
  modified_base (16)..(16): May be a locked nucleic acid thymidine
ccgggacaagtgcaat

SEQ ID NO: 39 - DNA, 16 bp, Artificial Sequence
  92a_LNA_16_16 miR-92 inhibitor
  misc_feature (1)..(16): May be joined by phosphorothioate bonds
  modified_base (1)..(1): May be a locked nucleic acid cytidine
  modified_base (3)..(3): May be a locked nucleic acid guanosine
  modified_base (6)..(6): May be a locked nucleic acid adenosine
  modified_base (8)..(9): May be a locked nucleic acid adenosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 40 - DNA, 16 bp, Artificial Sequence
  92a_LNA_16_17 miR-92 inhibitor
  misc_feature (1)..(16): May be joined by phosphorothioate bonds
  modified_base (1)..(1): May be a locked nucleic acid cytidine
  modified_base (3)..(3): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (12)..(12): May be a locked nucleic acid guanosine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 41 - DNA, 16 bp, Artificial Sequence
  92a_LNA_16_18 miR-92 inhibitor
  misc_feature (1)..(16): May be joined by phosphorothioate bonds
  modified_base (1)..(1): May be a locked nucleic acid cytidine
  modified_base (3)..(3): May be a locked nucleic acid guanosine
  modified_base (5)..(5): May be a locked nucleic acid guanosine
  modified_base (7)..(7): May be a locked nucleic acid cytidine
  modified_base (10)..(10): May be a locked nucleic acid guanosine
  modified_base (11)..(11): May be a locked nucleic acid thymidine
  modified_base (14)..(15): May be a locked nucleic acid adenosine
  modified_base (16)..(16): May be a locked nucleic acid thymidine
ccgggacaagtgcaat

SEQ ID NO: 42 - DNA, 16 bp, Artificial Sequence
  92a_LNA_16_19 miR-92 inhibitor
  misc_feature (1)..(16): May be joined by phosphorothioate bonds
  modified_base (1)..(1): May be a locked nucleic acid cytidine
  modified_base (3)..(3): May be a locked nucleic acid guanosine
  modified_base (6)..(6): May be a locked nucleic acid adenosine
  modified_base (8)..(8): May be a locked nucleic acid adenosine
  modified_base (11)..(11): May be a locked nucleic acid thymidine
  modified_base (13)..(13): May be a locked nucleic acid cytidine
  modified_base (14)..(15): May be a locked nucleic acid adenosine
  modified_base (16)..(16): May be a locked nucleic acid thymidine
ccgggacaagtgcaat

SEQ ID NO: 43 - DNA, 16 bp, Artificial Sequence
  92a_LNA_16_20 miR-92 inhibitor
  misc_feature (1)..(16): May be joined by phosphorothioate bonds
  modified_base (1)..(1): May be a locked nucleic acid cytidine
  modified_base (3)..(3): May be a locked nucleic acid guanosine
  modified_base (5)..(5): May be a locked nucleic acid guanosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 44 - DNA, 16 bp, Artificial Sequence
  92a_LNA_16_21 miR-92 inhibitor
  misc_feature (1)..(16): May be joined by phosphorothioate bonds
  modified_base (1)..(2): May be a locked nucleic acid cytidine
  modified_base (5)..(5): May be a locked nucleic acid guanosine
  modified_base (7)..(7): May be a locked nucleic acid cytidine
  modified_base (8)..(8): May be a locked nucleic acid adenosine
  modified_base (10)..(10): May be a locked nucleic acid guanosine
  modified_base (13)..(13): May be a locked nucleic acid cytidine
  modified_base (15)..(15): May be a locked nucleic acid adenosine
  modified_base (16)..(16): May be a locked nucleic acid thymidine
ccgggacaagtgcaat

SEQ ID NO: 45 - DNA, 16 bp, Artificial Sequence
  92a_LNA_16_22 miR-92 inhibitor
  misc_feature (1)..(16): May be joined by phosphorothioate bonds
  modified_base (1)..(1): May be a locked nucleic acid cytidine
  modified_base (3)..(4): May be a locked nucleic acid guanosine
  modified_base (7)..(7): May be a locked nucleic acid cytidine
  modified_base (9)..(9): May be a locked nucleic acid adenosine
  modified_base (10)..(10): May be a locked nucleic acid guanosine
  modified_base (12)..(12): May be a locked nucleic acid guanosine
  modified_base (14)..(14): May be a locked nucleic acid adenosine
  modified_base (16)..(16): May be a locked nucleic acid thymidine
ccgggacaagtgcaat

SEQ ID NO: 46 - DNA, 16 bp, Artificial Sequence
  92a_LNA_16_23 miR-92 inhibitor
  misc_feature (1)..(16): May be joined by phosphorothioate bonds
  modified_base (1)..(1): May be a locked nucleic acid cytidine
  modified_base (5)..(5): May be a locked nucleic acid guanosine
  modified_base (6)..(6): May be a locked nucleic acid adenosine
  modified_base (7)..(7): May be a locked nucleic acid cytidine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (15)..(15): May be a locked nucleic acid adenosine
  modified_base (16)..(16): May be a locked nucleic acid thymidine
ccgggacaagtgcaat

SEQ ID NO: 47 - DNA, 16 bp, Artificial Sequence
  92a_LNA_16_24 miR-92 inhibitor
  misc_feature (1)..(16): May be joined by phosphorothioate bonds
  modified_base (1)..(1): May be a locked nucleic acid cytidine
  modified_base (4)..(5): May be a locked nucleic acid guanosine
  modified_base (7)..(7): May be a locked nucleic acid cytidine
  modified_base (10)..(10): May be a locked nucleic acid guanosine
  modified_base (12)..(12): May be a locked nucleic acid guanosine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 48 - DNA, 16 bp, Artificial Sequence
  92a_LNA_16_25 miR-92 inhibitor
  misc_feature (1)..(16): May be joined by phosphorothioate bonds
  modified_base (1)..(1): May be a locked nucleic acid cytidine
  modified_base (4)..(5): May be a locked nucleic acid guanosine
  modified_base (7)..(7): May be a locked nucleic acid cytidine
  modified_base (8)..(8): May be a locked nucleic acid adenosine
  modified_base (10)..(10): May be a locked nucleic acid guanosine
  modified_base (12)..(12): May be a locked nucleic acid guanosine
  modified_base (14)..(14): May be a locked nucleic acid adenosine
  modified_base (16)..(16): May be a locked nucleic acid thymidine
ccgggacaagtgcaat

SEQ ID NO: 49 - DNA, 15 bp, Artificial Sequence
  92a_LNA_15_1 miR-92 inhibitor
  misc_feature (1)..(15): May be joined by phosphorothioate bonds
  modified_base (1)..(1): May be a locked nucleic acid cytidine
  modified_base (5)..(5): May be a locked nucleic acid adenosine
  modified_base (7)..(7): May be a locked nucleic acid adenosine
  modified_base (9)..(9): May be a locked nucleic acid guanosine
  modified_base (10)..(10): May be a locked nucleic acid thymidine
  modified_base (12)..(12): May be a locked nucleic acid cytidine
  modified_base (13)..(13): May be a locked nucleic acid adenosine
  modified_base (15)..(15): May be a locked nucleic acid thymidine
cgggacaagtgcaat

SEQ ID NO: 50 - DNA, 15 bp, Artificial Sequence
  92a_LNA_15_2 miR-92 inhibitor
  misc_feature (1)..(15): May be joined by phosphorothioate bonds
  modified_base (1)..(1): May be a locked nucleic acid cytidine
  modified_base (5)..(5): May be a locked nucleic acid adenosine
  modified_base (7)..(7): May be a locked nucleic acid adenosine
  modified_base (9)..(9): May be a locked nucleic acid guanosine
  modified_base (10)..(10): May be a locked nucleic acid thymidine
  modified_base (12)..(12): May be a locked nucleic acid cytidine
  modified_base (14)..(14): May be a locked nucleic acid adenosine
  modified_base (15)..(15): May be a locked nucleic acid thymidine
cgggacaagtgcaat

SEQ ID NO: 51 - DNA, 15 bp, Artificial Sequence
  92a_LNA_15_3 miR-92 inhibitor
  misc_feature (1)..(15): May be joined by phosphorothioate bonds
  modified_base (1)..(1): May be a locked nucleic acid cytidine
  modified_base (3)..(3): May be a locked nucleic acid guanosine
  modified_base (6)..(6): May be a locked nucleic acid cytidine
  modified_base (7)..(7): May be a locked nucleic acid adenosine
  modified_base (9)..(9): May be a locked nucleic acid guanosine
  modified_base (11)..(11): May be a locked nucleic acid guanosine
  modified_base (13)..(13): May be a locked nucleic acid adenosine
  modified_base (15)..(15): May be a locked nucleic acid thymidine
cgggacaagtgcaat

SEQ ID NO: 52 - DNA, 15 bp, Artificial Sequence
  92a_LNA_15_4 miR-92 inhibitor
  misc_feature (1)..(15): May be joined by phosphorothioate bonds
  modified_base (1)..(1): May be a locked nucleic acid cytidine
  modified_base (4)..(4): May be a locked nucleic acid guanosine
  modified_base (6)..(6): May be a locked nucleic acid cytidine
  modified_base (8)..(8): May be a locked nucleic acid adenosine
  modified_base (9)..(9): May be a locked nucleic acid guanosine
  modified_base (11)..(11): May be a locked nucleic acid guanosine
  modified_base (13)..(13): May be a locked nucleic acid adenosine
  modified_base (15)..(15): May be a locked nucleic acid thymidine
cgggacaagtgcaat

SEQ ID NO: 53 - DNA, 15 bp, Artificial Sequence
  92a_LNA_15_5 miR-92 inhibitor
  misc_feature (1)..(15): May be joined by phosphorothioate bonds
  modified_base (1)..(1): May be a locked nucleic acid cytidine
  modified_base (5)..(5): May be a locked nucleic acid adenosine
  modified_base (8)..(8): May be a locked nucleic acid adenosine
  modified_base (9)..(9): May be a locked nucleic acid guanosine
  modified_base (10)..(10): May be a locked nucleic acid thymidine
  modified_base (12)..(12): May be a locked nucleic acid cytidine
  modified_base (13)..(13): May be a locked nucleic acid adenosine
  modified_base (15)..(15): May be a locked nucleic acid thymidine
cgggacaagtgcaat

SEQ ID NO: 54 - DNA, 15 bp, Artificial Sequence
  92a_LNA_15_6 miR-92 inhibitor
  misc_feature (1)..(15): May be joined by phosphorothioate bonds
  modified_base (1)..(1): May be a locked nucleic acid cytidine
  modified_base (2)..(2): May be a locked nucleic acid guanosine
  modified_base (4)..(4): May be a locked nucleic acid guanosine
  modified_base (6)..(6): May be a locked nucleic acid cytidine
  modified_base (8)..(8): May be a locked nucleic acid adenosine
  modified_base (10)..(10): May be a locked nucleic acid thymidine
  modified_base (12)..(12): May be a locked nucleic acid cytidine
  modified_base (15)..(15): May be a locked nucleic acid thymidine
cgggacaagtgcaat

SEQ ID NO: 55 - DNA, 15 bp, Artificial Sequence
  92a_LNA_15_7 miR-92 inhibitor
  misc_feature (1)..(15): May be joined by phosphorothioate bonds
  modified_base (1)..(1): May be a locked nucleic acid cytidine
  modified_base (3)..(3): May be a locked nucleic acid guanosine
  modified_base (5)..(5): May be a locked nucleic acid adenosine
  modified_base (7)..(7): May be a locked nucleic acid adenosine
  modified_base (9)..(9): May be a locked nucleic acid guanosine
  modified_base (11)..(11): May be a locked nucleic acid guanosine
  modified_base (13)..(13): May be a locked nucleic acid adenosine
  modified_base (15)..(15): May be a locked nucleic acid thymidine
cgggacaagtgcaat

SEQ ID NO: 56 - DNA, 15 bp, Artificial Sequence
  92a_LNA_15_8 miR-92 inhibitor
  misc_feature (1)..(15): May be joined by phosphorothioate bonds
  modified_base (1)..(1): May be a locked nucleic acid cytidine
  modified_base (4)..(4): May be a locked nucleic acid guanosine
  modified_base (7)..(7): May be a locked nucleic acid adenosine
  modified_base (11)..(11): May be a locked nucleic acid guanosine
  modified_base (12)..(12): May be a locked nucleic acid cytidine
  modified_base (13)..(14): May be a locked nucleic acid adenosine
  modified_base (15)..(15): May be a locked nucleic acid thymidine
cgggacaagtgcaat

SEQ ID NO: 57 - DNA, 15 bp, Artificial Sequence
  92a_LNA_15_9 miR-92 inhibitor
  misc_feature (1)..(15): May be joined by phosphorothioate bonds
  modified_base (1)..(1): May be a locked nucleic acid cytidine
  modified_base (2)..(2): May be a locked nucleic acid guanosine
  modified_base (5)..(5): May be a locked nucleic acid adenosine
  modified_base (7)..(7): May be a locked nucleic acid adenosine
  modified_base (9)..(9): May be a locked nucleic acid guanosine
  modified_base (13)..(14): May be a locked nucleic acid adenosine
  modified_base (15)..(15): May be a locked nucleic acid thymidine
cgggacaagtgcaat

SEQ ID NO: 58 - DNA, 15 bp, Artificial Sequence
  92a_LNA_15_10 miR-92 inhibitor
  misc_feature (1)..(15): May be joined by phosphorothioate bonds
  modified_base (1)..(1): May be a locked nucleic acid cytidine
  modified_base (2)..(2): May be a locked nucleic acid guanosine
  modified_base (5)..(5): May be a locked nucleic acid adenosine
  modified_base (7)..(7): May be a locked nucleic acid adenosine
  modified_base (10)..(10): May be a locked nucleic acid thymidine
  modified_base (13)..(14): May be a locked nucleic acid adenosine
  modified_base (15)..(15): May be a locked nucleic acid thymidine
cgggacaagtgcaat

SEQ ID NO: 59 - DNA, 15 bp, Artificial Sequence
  92a_LNA_15_11 miR-92 inhibitor
  misc_feature (1)..(15): May be joined by phosphorothioate bonds
  modified_base (1)..(1): May be a locked nucleic acid cytidine
  modified_base (2)..(2): May be a locked nucleic acid guanosine
  modified_base (4)..(4): May be a locked nucleic acid guanosine
  modified_base (7)..(7): May be a locked nucleic acid adenosine
  modified_base (10)..(10): May be a locked nucleic acid thymidine
  modified_base (13)..(14): May be a locked nucleic acid adenosine
  modified_base (15)..(15): May be a locked nucleic acid thymidine
cgggacaagtgcaat

SEQ ID NO: 60 - DNA, 15 bp, Artificial Sequence
  92a_LNA_15_12 miR-92 inhibitor
  misc_feature (1)..(15): May be joined by phosphorothioate bonds
  modified_base (1)..(1): May be a locked nucleic acid cytidine
  modified_base (2)..(2): May be a locked nucleic acid guanosine
  modified_base (5)..(5): May be a locked nucleic acid adenosine
  modified_base (7)..(7): May be a locked nucleic acid adenosine
  modified_base (10)..(10): May be a locked nucleic acid thymidine
  modified_base (12)..(12): May be a locked nucleic acid cytidine
  modified_base (13)..(13): May be a locked nucleic acid adenosine
  modified_base (15)..(15): May be a locked nucleic acid thymidine
cgggacaagtgcaat

SEQ ID NO: 61 - DNA, 15 bp, Artificial Sequence
  92a_LNA_15_13 miR-92 inhibitor
  misc_feature (1)..(15): May be joined by phosphorothioate bonds
  modified_base (1)..(1): May be a locked nucleic acid cytidine
  modified_base (2)..(2): May be a locked nucleic acid guanosine
  modified_base (4)..(4): May be a locked nucleic acid guanosine
  modified_base (6)..(6): May be a locked nucleic acid cytidine
  modified_base (9)..(9): May be a locked nucleic acid guanosine
  modified_base (12)..(12): May be a locked nucleic acid cytidine
  modified_base (14)..(14): May be a locked nucleic acid adenosine
  modified_base (15)..(15): May be a locked nucleic acid thymidine
cgggacaagtgcaat

SEQ ID NO: 62 - DNA, 15 bp, Artificial Sequence
  92a_LNA_15_14 miR-92 inhibitor
  misc_feature (1)..(15): May be joined by phosphorothioate bonds
  modified_base (1)..(1): May be a locked nucleic acid cytidine
  modified_base (4)..(4): May be a locked nucleic acid guanosine
  modified_base (6)..(6): May be a locked nucleic acid cytidine
  modified_base (8)..(8): May be a locked nucleic acid adenosine
  modified_base (9)..(9): May be a locked nucleic acid guanosine
  modified_base (11)..(11): May be a locked nucleic acid guanosine
  modified_base (12)..(12): May be a locked nucleic acid cytidine
  modified_base (15)..(15): May be a locked nucleic acid thymidine
cgggacaagtgcaat

SEQ ID NO: 63 - DNA, 15 bp, Artificial Sequence
  92a_LNA_15_15 miR-92 inhibitor
  misc_feature (1)..(15): May be joined by phosphorothioate bonds
  modified_base (1)..(1): May be a locked nucleic acid cytidine
  modified_base (2)..(2): May be a locked nucleic acid guanosine
  modified_base (5)..(5): May be a locked nucleic acid adenosine
  modified_base (8)..(8): May be a locked nucleic acid adenosine
  modified_base (10)..(10): May be a locked nucleic acid thymidine
  modified_base (12)..(12): May be a locked nucleic acid cytidine
  modified_base (13)..(13): May be a locked nucleic acid adenosine
  modified_base (15)..(15): May be a locked nucleic acid thymidine
cgggacaagtgcaat

SEQ ID NO: 64 - DNA, 15 bp, Artificial Sequence
  92a_LNA_15_16 miR-92 inhibitor
  misc_feature (1)..(15): May be joined by phosphorothioate bonds
  modified_base (1)..(1): May be a locked nucleic acid cytidine
  modified_base (2)..(2): May be a locked nucleic acid guanosine
  modified_base (5)..(5): May be a locked nucleic acid adenosine
  modified_base (7)..(7): May be a locked nucleic acid adenosine
  modified_base (9)..(9): May be a locked nucleic acid guanosine
  modified_base (11)..(11): May be a locked nucleic acid guanosine
  modified_base (13)..(13): May be a locked nucleic acid adenosine
  modified_base (15)..(15): May be a locked nucleic acid thymidine
cgggacaagtgcaat

SEQ ID NO: 65 - DNA, 15 bp, Artificial Sequence
  92a_LNA_15_17 miR-92 inhibitor
  misc_feature (1)..(15): May be joined by phosphorothioate bonds
  modified_base (1)..(1): May be a locked nucleic acid cytidine
  modified_base (2)..(2): May be a locked nucleic acid guanosine
  modified_base (6)..(6): May be a locked nucleic acid cytidine
  modified_base (9)..(9): May be a locked nucleic acid guanosine
  modified_base (10)..(10): May be a locked nucleic acid thymidine
  modified_base (13)..(14): May be a locked nucleic acid adenosine
  modified_base (15)..(15): May be a locked nucleic acid thymidine
cgggacaagtgcaat

SEQ ID NO: 66 - DNA, 15 bp, Artificial Sequence
  92a_LNA_15_18 miR-92 inhibitor
  misc_feature (1)..(15): May be joined by phosphorothioate bonds
  modified_base (1)..(1): May be a locked nucleic acid cytidine
  modified_base (2)..(2): May be a locked nucleic acid guanosine
  modified_base (5)..(5): May be a locked nucleic acid adenosine
  modified_base (7)..(7): May be a locked nucleic acid adenosine
  modified_base (10)..(10): May be a locked nucleic acid thymidine
  modified_base (12)..(12): May be a locked nucleic acid cytidine
  modified_base (14)..(14): May be a locked nucleic acid adenosine
  modified_base (15)..(15): May be a locked nucleic acid thymidine
cgggacaagtgcaat

SEQ ID NO: 67 - DNA, 15 bp, Artificial Sequence
  92a_LNA_15_19 miR-92 inhibitor
  misc_feature (1)..(15): May be joined by phosphorothioate bonds
  modified_base (1)..(1): May be a locked nucleic acid cytidine
  modified_base (2)..(2): May be a locked nucleic acid guanosine
  modified_base (4)..(4): May be a locked nucleic acid guanosine
  modified_base (7)..(7): May be a locked nucleic acid adenosine
  modified_base (9)..(9): May be a locked nucleic acid guanosine
  modified_base (12)..(12): May be a locked nucleic acid cytidine
  modified_base (13)..(13): May be a locked nucleic acid adenosine
  modified_base (15)..(15): May be a locked nucleic acid thymidine
cgggacaagtgcaat

SEQ ID NO: 68 - DNA, 15 bp, Artificial Sequence
  92a_LNA_15_20 miR-92 inhibitor
  misc_feature (1)..(15): May be joined by phosphorothioate bonds
  modified_base (1)..(1): May be a locked nucleic acid cytidine
  modified_base (4)..(4): May be a locked nucleic acid guanosine
  modified_base (6)..(6): May be a locked nucleic acid cytidine
  modified_base (7)..(7): May be a locked nucleic acid adenosine
  modified_base (9)..(9): May be a locked nucleic acid guanosine
  modified_base (12)..(12): May be a locked nucleic acid cytidine
  modified_base (14)..(14): May be a locked nucleic acid adenosine
  modified_base (15)..(15): May be a locked nucleic acid thymidine
cgggacaagtgcaat

SEQ ID NO: 69 - DNA, 15 bp, Artificial Sequence
  92a_LNA_15_21 miR-92 inhibitor
  misc_feature (1)..(15): May be joined by phosphorothioate bonds
  modified_base (1)..(1): May be a locked nucleic acid cytidine
  modified_base (3)..(3): May be a locked nucleic acid guanosine
  modified_base (6)..(6): May be a locked nucleic acid cytidine
  modified_base (8)..(8): May be a locked nucleic acid adenosine
  modified_base (9)..(9): May be a locked nucleic acid guanosine
  modified_base (11)..(11): May be a locked nucleic acid guanosine
  modified_base (13)..(13): May be a locked nucleic acid adenosine
  modified_base (15)..(15): May be a locked nucleic acid thymidine
cgggacaagtgcaat

SEQ ID NO: 70 - DNA, 15 bp, Artificial Sequence
  92a_LNA_15_22 miR-92 inhibitor
  misc_feature (1)..(15): May be joined by phosphorothioate bonds
  modified_base (1)..(1): May be a locked nucleic acid cytidine
  modified_base (4)..(4): May be a locked nucleic acid guanosine
  modified_base (5)..(5): May be a locked nucleic acid adenosine
  modified_base (6)..(6): May be a locked nucleic acid cytidine
  modified_base (9)..(9): May be a locked nucleic acid guanosine
  modified_base (12)..(12): May be a locked nucleic acid cytidine
  modified_base (14)..(14): May be a locked nucleic acid adenosine
  modified_base (15)..(15): May be a locked nucleic acid thymidine
cgggacaagtgcaat

SEQ ID NO: 71 - DNA, 15 bp, Artificial Sequence
  92a_LNA_15_23 miR-92 inhibitor
  misc_feature (1)..(15): May be joined by phosphorothioate bonds
  modified_base (1)..(1): May be a locked nucleic acid cytidine
  modified_base (4)..(4): May be a locked nucleic acid guanosine
  modified_base (6)..(6): May be a locked nucleic acid cytidine
  modified_base (9)..(9): May be a locked nucleic acid guanosine
  modified_base (11)..(11): May be a locked nucleic acid guanosine
  modified_base (12)..(12): May be a locked nucleic acid cytidine
  modified_base (13)..(13): May be a locked nucleic acid adenosine
  modified_base (15)..(15): May be a locked nucleic acid thymidine
cgggacaagtgcaat

SEQ ID NO: 72 - DNA, 15 bp, Artificial Sequence
  92a_LNA_15_24 miR-92 inhibitor
  misc_feature (1)..(15): May be joined by phosphorothioate bonds
  modified_base (1)..(1): May be a locked nucleic acid cytidine
  modified_base (4)..(4): May be a locked nucleic acid guanosine
  modified_base (6)..(6): May be a locked nucleic acid cytidine
  modified_base (7)..(7): May be a locked nucleic acid adenosine
  modified_base (9)..(9): May be a locked nucleic acid guanosine
  modified_base (11)..(11): May be a locked nucleic acid guanosine
  modified_base (13)..(13): May be a locked nucleic acid adenosine
  modified_base (15)..(15): May be a locked nucleic acid thymidine
cgggacaagtgcaat

SEQ ID NO: 73 - DNA, 16 bp, Artificial Sequence
  miR-92 inhibitor
  misc_feature (1)..(16): May be joined by phosphorothioate bonds
  modified_base (1)..(1): May be a locked nucleic acid cytidine
  modified_base (3)..(5): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (13)..(13): May be a locked nucleic acid cytidine
  modified_base (16)..(16): May be a locked nucleic acid thymidine
ccgggacaagtgcaat

SEQ ID NO: 74 - DNA, 16 bp, Artificial Sequence
  miR-92 inhibitor
  misc_feature (1)..(16): May be joined by phosphorothioate bonds
  modified_base (1)..(1): May be a locked nucleic acid cytidine
  modified_base (3)..(4): May be a locked nucleic acid guanosine
  modified_base (6)..(6): May be a locked nucleic acid adenosine
  modified_base (8)..(9): May be a locked nucleic acid adenosine
  modified_base (10)..(10): May be a locked nucleic acid guanosine
  modified_base (13)..(13): May be a locked nucleic acid cytidine
  modified_base (15)..(15): May be a locked nucleic acid adenosine
ccgggacaagtgcaat

SEQ ID NO: 75 - DNA, 16 bp, Artificial Sequence
  miR-92 inhibitor
  misc_feature (1)..(16): May be joined by phosphorothioate bonds
  modified_base (1)..(1): May be a locked nucleic acid cytidine
  modified_base (3)..(5): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (13)..(13): May be a locked nucleic acid cytidine
  modified_base (15)..(15): May be a locked nucleic acid adenosine
ccgggacaagtgcaat

SEQ ID NO: 76 - DNA, 16 bp, Artificial Sequence
  miR-92 inhibitor
  misc_feature (1)..(16): May be joined by phosphorothioate bonds
  modified_base (1)..(1): May be a locked nucleic acid cytidine
  modified_base (5)..(5): May be a locked nucleic acid guanosine
  modified_base (6)..(6): May be a locked nucleic acid adenosine
  modified_base (9)..(9): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (15)..(15): May be a locked nucleic acid adenosine
  modified_base (16)..(16): May be a locked nucleic acid thymidine
ccgggacaagtgcaat

SEQ ID NO: 77 - DNA, 16 bp, Artificial Sequence
  miR-92 inhibitor
  misc_feature (1)..(16): May be joined by phosphorothioate bonds
  modified_base (1)..(1): May be a locked nucleic acid cytidine
  modified_base (5)..(5): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 78 - DNA, 16 bp, Artificial Sequence
  miR-92 inhibitor
  misc_feature (1)..(16): May be joined by phosphorothioate bonds
  modified_base (1)..(1): May be a locked nucleic acid cytidine
  modified_base (2)..(2): May be a O-methyl deoxycytidine
  modified_base (5)..(5): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (15)..(15): May be a locked nucleic acid adenosine
  modified_base (16)..(16): May be a locked nucleic acid thymidine
ccgggacaagtgcaat

SEQ ID NO: 79 - DNA, 16 bp, Artificial Sequence
  miR-92 inhibitor
  misc_feature (1)..(16): May be joined by phosphorothioate bonds
  modified_base (1)..(1): May be a locked nucleic acid cytidine
  modified_base (2)..(2): May be a O-methyl deoxycytidine
  modified_base (3)..(3): May be a locked nucleic acid guanosine
  modified_base (6)..(6): May be a locked nucleic acid adenosine
  modified_base (9)..(9): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 80 - DNA, 16 bp, Artificial Sequence
  miR-92 inhibitor
  misc_feature (1)..(16): May be joined by phosphorothioate bonds
  modified_base (1)..(1): May be a locked nucleic acid cytidine
  modified_base (2)..(2): May be a O-methyl deoxycytidine
  modified_base (3)..(3): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 81 - DNA, 14 bp, Artificial Sequence
  92a_LNA_14_1 miR-92 inhibitor
  misc_feature (1)..(14): May be joined by phosphorothioate bonds
  modified_base (1)..(1): May be a locked nucleic acid guanosine
  modified_base (4)..(4): May be a locked nucleic acid adenosine
  modified_base (6)..(6): May be a locked nucleic acid adenosine
  modified_base (8)..(8): May be a locked nucleic acid guanosine
  modified_base (9)..(9): May be a locked nucleic acid thymidine
  modified_base (11)..(11): May be a locked nucleic acid cytidine
  modified_base (12)..(12): May be a locked nucleic acid adenosine
  modified_base (14)..(14): May be a locked nucleic acid thymidine
gggacaagtgcaat

SEQ ID NO: 82 - DNA, 14 bp, Artificial Sequence
  92a_LNA_14_2 miR-92 inhibitor
  misc_feature (1)..(14): May be joined by phosphorothioate bonds
  modified_base (1)..(1): May be a locked nucleic acid guanosine
  modified_base (4)..(4): May be a locked nucleic acid adenosine
  modified_base (6)..(6): May be a locked nucleic acid adenosine
  modified_base (8)..(8): May be a locked nucleic acid guanosine
  modified_base (9)..(9): May be a locked nucleic acid thymidine
  modified_base (11)..(11): May be a locked nucleic acid cytidine
  modified_base (13)..(13): May be a locked nucleic acid adenosine
  modified_base (14)..(14): May be a locked nucleic acid thymidine
gggacaagtgcaat

SEQ ID NO: 83 - DNA, 14 bp, Artificial Sequence
  92a_LNA_14_3 miR-92 inhibitor
  misc_feature (1)..(14): May be joined by phosphorothioate bonds
  modified_base (1)..(2): May be a locked nucleic acid guanosine
  modified_base (5)..(5): May be a locked nucleic acid cytidine
  modified_base (6)..(6): May be a locked nucleic acid adenosine
  modified_base (8)..(8): May be a locked nucleic acid guanosine
  modified_base (10)..(10): May be a locked nucleic acid guanosine
  modified_base (12)..(12): May be a locked nucleic acid adenosine
  modified_base (14)..(14): May be a locked nucleic acid thymidine
gggacaagtgcaat

SEQ ID NO: 84 - DNA, 14 bp, Artificial Sequence
  92a_LNA_14_4 miR-92 inhibitor
  misc_feature (1)..(14): May be joined by phosphorothioate bonds
  modified_base (1)..(1): May be a locked nucleic acid guanosine
  modified_base (3)..(3): May be a locked nucleic acid guanosine
  modified_base (5)..(5): May be a locked nucleic acid cytidine
  modified_base (7)..(7): May be a locked nucleic acid adenosine
  modified_base (8)..(8): May be a locked nucleic acid guanosine
  modified_base (10)..(10): May be a locked nucleic acid guanosine
  modified_base (12)..(12): May be a locked nucleic acid adenosine
  modified_base (14)..(14): May be a locked nucleic acid thymidine
gggacaagtgcaat

SEQ ID NO: 85 - DNA, 14 bp, Artificial Sequence
  92a_LNA_14_5 miR-92 inhibitor
  misc_feature (1)..(14): May be joined by phosphorothioate bonds
  modified_base (1)..(1): May be a locked nucleic acid guanosine
  modified_base (4)..(4): May be a locked nucleic acid adenosine
  modified_base (7)..(7): May be a locked nucleic acid adenosine
  modified_base (8)..(8): May be a locked nucleic acid guanosine
  modified_base (9)..(9): May be a locked nucleic acid thymidine
  modified_base (11)..(11): May be a locked nucleic acid cytidine
  modified_base (12)..(12): May be a locked nucleic acid adenosine
  modified_base (14)..(14): May be a locked nucleic acid thymidine
gggacaagtgcaat

SEQ ID NO: 86 - DNA, 14 bp, Artificial Sequence
  92a_LNA_14_6 miR-92 inhibitor
  misc_feature (1)..(14): May be joined by phosphorothioate bonds
  modified_base (1)..(1): May be a locked nucleic acid guanosine
  modified_base (3)..(3): May be a locked nucleic acid guanosine
  modified_base (5)..(5): May be a locked nucleic acid cytidine
  modified_base (7)..(7): May be a locked nucleic acid adenosine
  modified_base (9)..(9): May be a locked nucleic acid thymidine
  modified_base (11)..(11): May be a locked nucleic acid cytidine
  modified_base (14)..(14): May be a locked nucleic acid thymidine
gggacaagtgcaat

SEQ ID NO: 87 - DNA, 14 bp, Artificial Sequence
  92a_LNA_14_7 miR-92 inhibitor
  misc_feature (1)..(14): May be joined by phosphorothioate bonds
  modified_base (1)..(2): May be a locked nucleic acid guanosine
  modified_base (4)..(4): May be a locked nucleic acid adenosine
  modified_base (6)..(6): May be a locked nucleic acid adenosine
  modified_base (8)..(8): May be a locked nucleic acid guanosine
  modified_base (10)..(10): May be a locked nucleic acid guanosine
  modified_base (12)..(12): May be a locked nucleic acid adenosine
  modified_base (14)..(14): May be a locked nucleic acid thymidine
gggacaagtgcaat

SEQ ID NO: 88 - DNA, 14 bp, Artificial Sequence
  92a_LNA_14_8 miR-92 inhibitor
  misc_feature (1)..(14): May be joined by phosphorothioate bonds
  modified_base (1)..(1): May be a locked nucleic acid guanosine
  modified_base (3)..(3): May be a locked nucleic acid guanosine
  modified_base (6)..(6): May be a locked nucleic acid adenosine
  modified_base (10)..(10): May be a locked nucleic acid guanosine
  modified_base (11)..(11): May be a locked nucleic acid cytidine
  modified_base (12)..(13): May be a locked nucleic acid adenosine
  modified_base (14)..(14): May be a locked nucleic acid thymidine
gggacaagtgcaat

SEQ ID NO: 89 - DNA, 14 bp, Artificial Sequence
  92a_LNA_14_9 miR-92 inhibitor
  misc_feature (1)..(14): May be joined by phosphorothioate bonds
  modified_base (1)..(1): May be a locked nucleic acid guanosine
  modified_base (4)..(4): May be a locked nucleic acid adenosine
  modified_base (6)..(6): May be a locked nucleic acid adenosine
  modified_base (8)..(8): May be a locked nucleic acid guanosine
  modified_base (12)..(13): May be a locked nucleic acid adenosine
  modified_base (14)..(14): May be a locked nucleic acid thymidine
gggacaagtgcaat

SEQ ID NO: 90 - DNA, 14 bp, Artificial Sequence
  92a_LNA_14_10 miR-92 inhibitor
  misc_feature (1)..(14): May be joined by phosphorothioate bonds
  modified_base (1)..(1): May be a locked nucleic acid guanosine
  modified_base (4)..(4): May be a locked nucleic acid adenosine
  modified_base (6)..(6): May be a locked nucleic acid adenosine
  modified_base (9)..(9): May be a locked nucleic acid thymidine
  modified_base (12)..(13): May be a locked nucleic acid adenosine
  modified_base (14)..(14): May be a locked nucleic acid thymidine
gggacaagtgcaat

SEQ ID NO: 91 - DNA, 14 bp, Artificial Sequence
  92a_LNA_14_11 miR-92 inhibitor
  misc_feature (1)..(14): May be joined by phosphorothioate bonds
  modified_base (1)..(1): May be a locked nucleic acid guanosine
  modified_base (3)..(3): May be a locked nucleic acid guanosine
  modified_base (6)..(6): May be a locked nucleic acid adenosine
  modified_base (9)..(9): May be a locked nucleic acid thymidine
  modified_base (12)..(13): May be a locked nucleic acid adenosine
  modified_base (14)..(14): May be a locked nucleic acid thymidine
gggacaagtgcaat

SEQ ID NO: 92 - DNA, 14 bp, Artificial Sequence
  92a_LNA_14_12 miR-92 inhibitor
  misc_feature (1)..(14): May be joined by phosphorothioate bonds
  modified_base (1)..(1): May be a locked nucleic acid guanosine
  modified_base (4)..(4): May be a locked nucleic acid adenosine
  modified_base (6)..(6): May be a locked nucleic acid adenosine
  modified_base (9)..(9): May be a locked nucleic acid thymidine
  modified_base (11)..(11): May be a locked nucleic acid cytidine
  modified_base (12)..(12): May be a locked nucleic acid adenosine
  modified_base (14)..(14): May be a locked nucleic acid thymidine
gggacaagtgcaat

SEQ ID NO: 93 - DNA, 14 bp, Artificial Sequence
  92a_LNA_14_13 miR-92 inhibitor
  misc_feature (1)..(14): May be joined by phosphorothioate bonds
  modified_base (1)..(1): May be a locked nucleic acid guanosine
  modified_base (3)..(3): May be a locked nucleic acid guanosine
  modified_base (5)..(5): May be a locked nucleic acid cytidine
  modified_base (8)..(8): May be a locked nucleic acid guanosine
  modified_base (11)..(11): May be a locked nucleic acid cytidine
  modified_base (13)..(13): May be a locked nucleic acid adenosine
  modified_base (14)..(14): May be a locked nucleic acid thymidine
gggacaagtgcaat

SEQ ID NO: 94 - DNA, 14 bp, Artificial Sequence
  92a_LNA_14_14 miR-92 inhibitor
  misc_feature (1)..(14): May be joined by phosphorothioate bonds
  modified_base (1)..(1): May be a locked nucleic acid guanosine
  modified_base (3)..(3): May be a locked nucleic acid guanosine
  modified_base (5)..(5): May be a locked nucleic acid cytidine
  modified_base (7)..(7): May be a locked nucleic acid adenosine
  modified_base (8)..(8): May be a locked nucleic acid guanosine
  modified_base (10)..(10): May be a locked nucleic acid guanosine
  modified_base (11)..(11): May be a locked nucleic acid cytidine
  modified_base (14)..(14): May be a locked nucleic acid thymidine
gggacaagtgcaat

SEQ ID NO: 95 - DNA, 14 bp, Artificial Sequence
  92a_LNA_14_15 miR-92 inhibitor
  misc_feature (1)..(14): May be joined by phosphorothioate bonds
  modified_base (1)..(1): May be a locked nucleic acid guanosine
  modified_base (4)..(4): May be a locked nucleic acid adenosine
  modified_base (7)..(7): May be a locked nucleic acid adenosine
  modified_base (9)..(9): May be a locked nucleic acid thymidine
  modified_base (11)..(11): May be a locked nucleic acid cytidine
  modified_base (12)..(12): May be a locked nucleic acid adenosine
  modified_base (14)..(14): May be a locked nucleic acid thymidine
gggacaagtgcaat

SEQ ID NO: 96 - DNA, 14 bp, Artificial Sequence
  92a_LNA_14_16 miR-92 inhibitor
  misc_feature (1)..(14): May be joined by phosphorothioate bonds
  modified_base (1)..(1): May be a locked nucleic acid guanosine
  modified_base (4)..(4): May be a locked nucleic acid adenosine
  modified_base (6)..(6): May be a locked nucleic acid adenosine
  modified_base (8)..(8): May be a locked nucleic acid guanosine
  modified_base (10)..(10): May be a locked nucleic acid guanosine
  modified_base (12)..(12): May be a locked nucleic acid adenosine
  modified_base (14)..(14): May be a locked nucleic acid thymidine
gggacaagtgcaat

SEQ ID NO: 97 - DNA, 14 bp, Artificial Sequence
  92a_LNA_14_17 miR-92 inhibitor
  misc_feature (1)..(14): May be joined by phosphorothioate bonds
  modified_base (1)..(1): May be a locked nucleic acid guanosine
  modified_base (5)..(5): May be a locked nucleic acid cytidine
  modified_base (8)..(8): May be a locked nucleic acid guanosine
  modified_base (9)..(9): May be a locked nucleic acid thymidine
  modified_base (12)..(13): May be a locked nucleic acid adenosine
  modified_base (14)..(14): May be a locked nucleic acid thymidine
gggacaagtgcaat

SEQ ID NO: 98 - DNA, 14 bp, Artificial Sequence
  92a_LNA_14_18 miR-92 inhibitor
  misc_feature (1)..(14): May be joined by phosphorothioate bonds
  modified_base (1)..(1): May be a locked nucleic acid guanosine
  modified_base (4)..(4): May be a locked nucleic acid adenosine
  modified_base (6)..(6): May be a locked nucleic acid adenosine
  modified_base (9)..(9): May be a locked nucleic acid thymidine
  modified_base (11)..(11): May be a locked nucleic acid cytidine
  modified_base (13)..(13): May be a locked nucleic acid adenosine
  modified_base (14)..(14): May be a locked nucleic acid thymidine
gggacaagtgcaat

SEQ ID NO: 99 - DNA, 14 bp, Artificial Sequence
  92a_LNA_14_19 miR-92 inhibitor
  misc_feature (1)..(14): May be joined by phosphorothioate bonds
  modified_base (1)..(1): May be a locked nucleic acid guanosine
  modified_base (3)..(3): May be a locked nucleic acid guanosine
  modified_base (6)..(6): May be a locked nucleic acid adenosine
  modified_base (8)..(8): May be a locked nucleic acid guanosine
  modified_base (11)..(11): May be a locked nucleic acid cytidine
  modified_base (12)..(12): May be a locked nucleic acid adenosine
  modified_base (14)..(14): May be a locked nucleic acid thymidine
gggacaagtgcaat

SEQ ID NO: 100 - DNA, 14 bp, Artificial Sequence
  92a_LNA_14_20 miR-92 inhibitor
  misc_feature (1)..(14): May be joined by phosphorothioate bonds
  modified_base (1)..(1): May be a locked nucleic acid guanosine
  modified_base (3)..(3): May be a locked nucleic acid guanosine
  modified_base (5)..(5): May be a locked nucleic acid cytidine
  modified_base (6)..(6): May be a locked nucleic acid adenosine
  modified_base (8)..(8): May be a locked nucleic acid guanosine
  modified_base (11)..(11): May be a locked nucleic acid cytidine
  modified_base (13)..(13): May be a locked nucleic acid adenosine
  modified_base (14)..(14): May be a locked nucleic acid thymidine
gggacaagtgcaat

SEQ ID NO: 101 - DNA, 14 bp, Artificial Sequence
  92a_LNA_14_21 miR-92 inhibitor
  misc_feature (1)..(14): May be joined by phosphorothioate bonds
  modified_base (1)..(2): May be a locked nucleic acid guanosine
  modified_base (5)..(5): May be a locked nucleic acid cytidine
  modified_base (7)..(7): May be a locked nucleic acid adenosine
  modified_base (8)..(8): May be a locked nucleic acid guanosine
  modified_base (10)..(10): May be a locked nucleic acid guanosine
  modified_base (12)..(12): May be a locked nucleic acid adenosine
  modified_base (14)..(14): May be a locked nucleic acid thymidine
gggacaagtgcaat

SEQ ID NO: 102 - DNA, 14 bp, Artificial Sequence
  92a_LNA_14_22 miR-92 inhibitor
  misc_feature (1)..(14): May be joined by phosphorothioate bonds
  modified_base (1)..(1): May be a locked nucleic acid guanosine
  modified_base (3)..(3): May be a locked nucleic acid guanosine
  modified_base (4)..(4): May be a locked nucleic acid adenosine
  modified_base (5)..(5): May be a locked nucleic acid cytidine
  modified_base (8)..(8): May be a locked nucleic acid guanosine
  modified_base (11)..(11): May be a locked nucleic acid cytidine
  modified_base (13)..(13): May be a locked nucleic acid adenosine
  modified_base (14)..(14): May be a locked nucleic acid thymidine
gggacaagtgcaat

SEQ ID NO: 103 - DNA, 14 bp, Artificial Sequence
  92a_LNA_14_23 miR-92 inhibitor
  misc_feature (1)..(14): May be joined by phosphorothioate bonds
  modified_base (1)..(1): May be a locked nucleic acid guanosine
  modified_base (3)..(3): May be a locked nucleic acid guanosine
  modified_base (5)..(5): May be a locked nucleic acid cytidine
  modified_base (8)..(8): May be a locked nucleic acid guanosine
  modified_base (10)..(10): May be a locked nucleic acid guanosine
  modified_base (11)..(11): May be a locked nucleic acid cytidine
  modified_base (12)..(12): May be a locked nucleic acid adenosine
  modified_base (14)..(14): May be a locked nucleic acid thymidine
gggacaagtgcaat

SEQ ID NO: 104 - DNA, 14 bp, Artificial Sequence
  92a_LNA_14_24 miR-92 inhibitor
  misc_feature (1)..(14): May be joined by phosphorothioate bonds
  modified_base (1)..(1): May be a locked nucleic acid guanosine
  modified_base (3)..(3): May be a locked nucleic acid guanosine
  modified_base (5)..(5): May be a locked nucleic acid cytidine
  modified_base (6)..(6): May be a locked nucleic acid adenosine
  modified_base (8)..(8): May be a locked nucleic acid guanosine
  modified_base (10)..(10): May be a locked nucleic acid guanosine
  modified_base (12)..(12): May be a locked nucleic acid adenosine
  modified_base (14)..(14): May be a locked nucleic acid thymidine
gggacaagtgcaat

SEQ ID NO: 105 - DNA, 13 bp, Artificial Sequence
  92a_LNA_13_1 miR-92 inhibitor
  misc_feature (1)..(13): May be joined by phosphorothioate bonds
  modified_base (1)..(1): May be a locked nucleic acid guanosine
  modified_base (3)..(3): May be a locked nucleic acid adenosine
  modified_base (5)..(5): May be a locked nucleic acid adenosine
  modified_base (7)..(7): May be a locked nucleic acid guanosine
  modified_base (8)..(8): May be a locked nucleic acid thymidine
  modified_base (10)..(10): May be a locked nucleic acid cytidine
  modified_base (11)..(11): May be a locked nucleic acid adenosine
  modified_base (13)..(13): May be a locked nucleic acid thymidine
ggacaagtgcaat

SEQ ID NO: 106 - DNA, 13 bp, Artificial Sequence
  92a_LNA_13_2 miR-92 inhibitor
  misc_feature (1)..(13): May be joined by phosphorothioate bonds
  modified_base (1)..(1): May be a locked nucleic acid guanosine
  modified_base (3)..(3): May be a locked nucleic acid adenosine
  modified_base (5)..(5): May be a locked nucleic acid adenosine
  modified_base (7)..(7): May be a locked nucleic acid guanosine
  modified_base (8)..(8): May be a locked nucleic acid thymidine
  modified_base (10)..(10): May be a locked nucleic acid cytidine
  modified_base (12)..(12): May be a locked nucleic acid adenosine
  modified_base (13)..(13): May be a locked nucleic acid thymidine
ggacaagtgcaat

SEQ ID NO: 107 - DNA, 13 bp, Artificial Sequence
  92a_LNA_13_3 miR-92 inhibitor
  misc_feature (1)..(13): May be joined by phosphorothioate bonds
  modified_base (1)..(1): May be a locked nucleic acid guanosine
  modified_base (4)..(4): May be a locked nucleic acid cytidine
  modified_base (5)..(5): May be a locked nucleic acid adenosine
  modified_base (7)..(7): May be a locked nucleic acid guanosine
  modified_base (9)..(9): May be a locked nucleic acid guanosine
  modified_base (11)..(11): May be a locked nucleic acid adenosine
  modified_base (13)..(13): May be a locked nucleic acid thymidine
ggacaagtgcaat

SEQ ID NO: 108 - DNA, 13 bp, Artificial Sequence
  92a_LNA_13_4 miR-92 inhibitor
  misc_feature (1)..(13): May be joined by phosphorothioate bonds
  modified_base (1)..(2): May be a locked nucleic acid guanosine
  modified_base (4)..(4): May be a locked nucleic acid cytidine
  modified_base (6)..(6): May be a locked nucleic acid adenosine
  modified_base (7)..(7): May be a locked nucleic acid guanosine
  modified_base (9)..(9): May be a locked nucleic acid guanosine
  modified_base (11)..(11): May be a locked nucleic acid adenosine
  modified_base (13)..(13): May be a locked nucleic acid thymidine
ggacaagtgcaat

SEQ ID NO: 109 - DNA, 13 bp, Artificial Sequence
  92a_LNA_13_5 miR-92 inhibitor
  misc_feature (1)..(13): May be joined by phosphorothioate bonds
  modified_base (1)..(1): May be a locked nucleic acid guanosine
  modified_base (3)..(3): May be a locked nucleic acid adenosine
  modified_base (6)..(6): May be a locked nucleic acid adenosine
  modified_base (7)..(7): May be a locked nucleic acid guanosine
  modified_base (8)..(8): May be a locked nucleic acid thymidine
  modified_base (10)..(10): May be a locked nucleic acid cytidine
  modified_base (11)..(11): May be a locked nucleic acid adenosine
  modified_base (13)..(13): May be a locked nucleic acid thymidine
ggacaagtgcaat

SEQ ID NO: 110 - DNA, 13 bp, Artificial Sequence
  92a_LNA_13_6 miR-92 inhibitor
  misc_feature (1)..(13): May be joined by phosphorothioate bonds
  modified_base (1)..(2): May be a locked nucleic acid guanosine
  modified_base (4)..(4): May be a locked nucleic acid cytidine
  modified_base (6)..(6): May be a locked nucleic acid adenosine
  modified_base (8)..(8): May be a locked nucleic acid thymidine
  modified_base (10)..(10): May be a locked nucleic acid cytidine
  modified_base (13)..(13): May be a locked nucleic acid thymidine
ggacaagtgcaat

SEQ ID NO: 111 - DNA, 13 bp, Artificial Sequence
  92a_LNA_13_7 miR-92 inhibitor
  misc_feature (1)..(13): May be joined by phosphorothioate bonds
  modified_base (1)..(1): May be a locked nucleic acid guanosine
  modified_base (3)..(3): May be a locked nucleic acid adenosine
  modified_base (5)..(5): May be a locked nucleic acid adenosine
  modified_base (7)..(7): May be a locked nucleic acid guanosine
  modified_base (9)..(9): May be a locked nucleic acid guanosine
  modified_base (11)..(11): May be a locked nucleic acid adenosine
  modified_base (13)..(13): May be a locked nucleic acid thymidine
ggacaagtgcaat

SEQ ID NO: 112 - DNA, 13 bp, Artificial Sequence
  92a_LNA_13_8 miR-92 inhibitor
  misc_feature (1)..(13): May be joined by phosphorothioate bonds
  modified_base (1)..(2): May be a locked nucleic acid guanosine
  modified_base (5)..(5): May be a locked nucleic acid adenosine
  modified_base (9)..(9): May be a locked nucleic acid guanosine
  modified_base (10)..(10): May be a locked nucleic acid cytidine
  modified_base (11)..(12): May be a locked nucleic acid adenosine
  modified_base (13)..(13): May be a locked nucleic acid thymidine
ggacaagtgcaat

SEQ ID NO: 113 - DNA, 13 bp, Artificial Sequence
  92a_LNA_13_9 miR-92 inhibitor
  misc_feature (1)..(13): May be joined by phosphorothioate bonds
  modified_base (1)..(1): May be a locked nucleic acid guanosine
  modified_base (3)..(3): May be a locked nucleic acid adenosine
  modified_base (5)..(5): May be a locked nucleic acid adenosine
  modified_base (7)..(7): May be a locked nucleic acid guanosine
  modified_base (11)..(12): May be a locked nucleic acid adenosine
  modified_base (13)..(13): May be a locked nucleic acid thymidine
ggacaagtgcaat

SEQ ID NO: 114 - DNA, 13 bp, Artificial Sequence
  92a_LNA_13_10 miR-92 inhibitor
  misc_feature (1)..(13): May be joined by phosphorothioate bonds
  modified_base (1)..(1): May be a locked nucleic acid guanosine
  modified_base (3)..(3): May be a locked nucleic acid adenosine
  modified_base (5)..(5): May be a locked nucleic acid adenosine
  modified_base (8)..(8): May be a locked nucleic acid thymidine
  modified_base (11)..(12): May be a locked nucleic acid adenosine
  modified_base (13)..(13): May be a locked nucleic acid thymidine
ggacaagtgcaat

SEQ ID NO: 115 - DNA, 13 bp, Artificial Sequence
  92a_LNA_13_11 miR-92 inhibitor
  misc_feature (1)..(13): May be joined by phosphorothioate bonds
  modified_base (1)..(2): May be a locked nucleic acid guanosine
  modified_base (5)..(5): May be a locked nucleic acid adenosine
  modified_base (8)..(8): May be a locked nucleic acid thymidine
  modified_base (11)..(12): May be a locked nucleic acid adenosine
  modified_base (13)..(13): May be a locked nucleic acid thymidine
ggacaagtgcaat

SEQ ID NO: 116 - DNA, 13 bp, Artificial Sequence
  92a_LNA_13_12 miR-92 inhibitor
  misc_feature (1)..(13): May be joined by phosphorothioate bonds
  modified_base (1)..(1): May be a locked nucleic acid guanosine
  modified_base (3)..(3): May be a locked nucleic acid adenosine
  modified_base (5)..(5): May be a locked nucleic acid adenosine
  modified_base (8)..(8): May be a locked nucleic acid thymidine
  modified_base (10)..(10): May be a locked nucleic acid cytidine
  modified_base (11)..(11): May be a locked nucleic acid adenosine
  modified_base (13)..(13): May be a locked nucleic acid thymidine
ggacaagtgcaat

SEQ ID NO: 117 - DNA, 13 bp, Artificial Sequence
  92a_LNA_13_13 miR-92 inhibitor
  misc_feature (1)..(13): May be joined by phosphorothioate bonds
  modified_base (1)..(2): May be a locked nucleic acid guanosine
  modified_base (4)..(4): May be a locked nucleic acid cytidine
  modified_base (7)..(7): May be a locked nucleic acid guanosine
  modified_base (10)..(10): May be a locked nucleic acid cytidine
  modified_base (12)..(12): May be a locked nucleic acid adenosine
  modified_base (13)..(13): May be a locked nucleic acid thymidine
ggacaagtgcaat

SEQ ID NO: 118 - DNA, 13 bp, Artificial Sequence
  92a_LNA_13_14 miR-92 inhibitor
  misc_feature (1)..(13): May be joined by phosphorothioate bonds
  modified_base (1)..(2): May be a locked nucleic acid guanosine
  modified_base (4)..(4): May be a locked nucleic acid cytidine
  modified_base (6)..(6): May be a locked nucleic acid adenosine
  modified_base (7)..(7): May be a locked nucleic acid guanosine
  modified_base (9)..(9): May be a locked nucleic acid guanosine
  modified_base (10)..(10): May be a locked nucleic acid cytidine
  modified_base (13)..(13): May be a locked nucleic acid thymidine
ggacaagtgcaat

SEQ ID NO: 119 - DNA, 13 bp, Artificial Sequence
  92a_LNA_13_15 miR-92 inhibitor
  misc_feature (1)..(13): May be joined by phosphorothioate bonds
  modified_base (1)..(1): May be a locked nucleic acid guanosine
  modified_base (3)..(3): May be a locked nucleic acid adenosine
  modified_base (6)..(6): May be a locked nucleic acid adenosine
  modified_base (8)..(8): May be a locked nucleic acid thymidine
  modified_base (10)..(10): May be a locked nucleic acid cytidine
  modified_base (11)..(11): May be a locked nucleic acid adenosine
  modified_base (13)..(13): May be a locked nucleic acid thymidine
ggacaagtgcaat

SEQ ID NO: 120 - DNA, 13 bp, Artificial Sequence
  92a_LNA_13_16 miR-92 inhibitor
  misc_feature (1)..(13): May be joined by phosphorothioate bonds
  modified_base (1)..(1): May be a locked nucleic acid guanosine
  modified_base (3)..(3): May be a locked nucleic acid adenosine
  modified_base (5)..(5): May be a locked nucleic acid adenosine
  modified_base (7)..(7): May be a locked nucleic acid guanosine
  modified_base (9)..(9): May be a locked nucleic acid guanosine
  modified_base (11)..(11): May be a locked nucleic acid adenosine
  modified_base (13)..(13): May be a locked nucleic acid thymidine
ggacaagtgcaat

SEQ ID NO: 121 - DNA, 13 bp, Artificial Sequence
  92a_LNA_13_17 miR-92 inhibitor
  misc_feature (1)..(13): May be joined by phosphorothioate bonds
  modified_base (1)..(1): May be a locked nucleic acid guanosine
  modified_base (4)..(4): May be a locked nucleic acid cytidine
  modified_base (7)..(7): May be a locked nucleic acid guanosine
  modified_base (8)..(8): May be a locked nucleic acid thymidine
  modified_base (11)..(12): May be a locked nucleic acid adenosine
  modified_base (13)..(13): May be a locked nucleic acid thymidine
ggacaagtgcaat

SEQ ID NO: 122 - DNA, 13 bp, Artificial Sequence
  92a_LNA_13_18 miR-92 inhibitor
  misc_feature (1)..(13): May be joined by phosphorothioate bonds
  modified_base (1)..(1): May be a locked nucleic acid guanosine
  modified_base (3)..(3): May be a locked nucleic acid adenosine
  modified_base (5)..(5): May be a locked nucleic acid adenosine
  modified_base (8)..(8): May be a locked nucleic acid thymidine
  modified_base (10)..(10): May be a locked nucleic acid cytidine
  modified_base (12)..(12): May be a locked nucleic acid adenosine
  modified_base (13)..(13): May be a locked nucleic acid thymidine
ggacaagtgcaat

SEQ ID NO: 123 - DNA, 13 bp, Artificial Sequence
  92a_LNA_13_19 miR-92 inhibitor
  misc_feature (1)..(13): May be joined by phosphorothioate bonds
  modified_base (1)..(2): May be a locked nucleic acid guanosine
  modified_base (5)..(5): May be a locked nucleic acid adenosine
  modified_base (7)..(7): May be a locked nucleic acid guanosine
  modified_base (10)..(10): May be a locked nucleic acid cytidine
  modified_base (11)..(11): May be a locked nucleic acid adenosine
  modified_base (13)..(13): May be a locked nucleic acid thymidine
ggacaagtgcaat

SEQ ID NO: 124 - DNA, 13 bp, Artificial Sequence
  92a_LNA_13_20 miR-92 inhibitor
  misc_feature (1)..(13): May be joined by phosphorothioate bonds
  modified_base (1)..(2): May be a locked nucleic acid guanosine
  modified_base (4)..(4): May be a locked nucleic acid cytidine
  modified_base (5)..(5): May be a locked nucleic acid adenosine
  modified_base (7)..(7): May be a locked nucleic acid guanosine
  modified_base (10)..(10): May be a locked nucleic acid cytidine
  modified_base (12)..(12): May be a locked nucleic acid adenosine
  modified_base (13)..(13): May be a locked nucleic acid thymidine
ggacaagtgcaat

SEQ ID NO: 125 - DNA, 13 bp, Artificial Sequence
  92a_LNA_13_21 miR-92 inhibitor
  misc_feature (1)..(13): May be joined by phosphorothioate bonds
  modified_base (1)..(1): May be a locked nucleic acid guanosine
  modified_base (4)..(4): May be a locked nucleic acid cytidine
  modified_base (6)..(6): May be a locked nucleic acid adenosine
  modified_base (7)..(7): May be a locked nucleic acid guanosine
  modified_base (9)..(9): May be a locked nucleic acid guanosine
  modified_base (11)..(11): May be a locked nucleic acid adenosine
  modified_base (13)..(13): May be a locked nucleic acid thymidine
ggacaagtgcaat

SEQ ID NO: 126 - DNA, 13 bp, Artificial Sequence
  92a_LNA_13_22 miR-92 inhibitor
  misc_feature (1)..(13): May be joined by phosphorothioate bonds
  modified_base (1)..(2): May be a locked nucleic acid guanosine
  modified_base (3)..(3): May be a locked nucleic acid adenosine
  modified_base (4)..(4): May be a locked nucleic acid cytidine
  modified_base (7)..(7): May be a locked nucleic acid guanosine
  modified_base (10)..(10): May be a locked nucleic acid cytidine
  modified_base (12)..(12): May be a locked nucleic acid adenosine
  modified_base (13)..(13): May be a locked nucleic acid thymidine
ggacaagtgcaat

SEQ ID NO: 127 - DNA, 13 bp, Artificial Sequence
  92a_LNA_13_23 miR-92 inhibitor
  misc_feature (1)..(13): May be joined by phosphorothioate bonds
  modified_base (1)..(2): May be a locked nucleic acid guanosine
  modified_base (4)..(4): May be a locked nucleic acid cytidine
  modified_base (7)..(7): May be a locked nucleic acid guanosine
  modified_base (9)..(9): May be a locked nucleic acid guanosine
  modified_base (10)..(10): May be a locked nucleic acid cytidine
  modified_base (11)..(11): May be a locked nucleic acid adenosine
  modified_base (13)..(13): May be a locked nucleic acid thymidine
ggacaagtgcaat

SEQ ID NO: 128 - DNA, 13 bp, Artificial Sequence
  92a_LNA_13_24 miR-92 inhibitor
  misc_feature (1)..(13): May be joined by phosphorothioate bonds
  modified_base (1)..(2): May be a locked nucleic acid guanosine
  modified_base (4)..(4): May be a locked nucleic acid cytidine
  modified_base (5)..(5): May be a locked nucleic acid adenosine
  modified_base (7)..(7): May be a locked nucleic acid guanosine
  modified_base (9)..(9): May be a locked nucleic acid guanosine
  modified_base (11)..(11): May be a locked nucleic acid adenosine
  modified_base (13)..(13): May be a locked nucleic acid thymidine
ggacaagtgcaat

SEQ ID NO: 129 - DNA, 12 bp, Artificial Sequence
  92a_LNA_12_1 miR-92 inhibitor
  misc_feature (1)..(12): May be joined by phosphorothioate bonds
  modified_base (1)..(1): May be a locked nucleic acid guanosine
  modified_base (2)..(2): May be a locked nucleic acid adenosine
  modified_base (4)..(4): May be a locked nucleic acid adenosine
  modified_base (6)..(6): May be a locked nucleic acid guanosine
  modified_base (7)..(7): May be a locked nucleic acid thymidine
  modified_base (9)..(9): May be a locked nucleic acid cytidine
  modified_base (10)..(10): May be a locked nucleic acid adenosine
  modified_base (12)..(12): May be a locked nucleic acid thymidine
gacaagtgcaat

SEQ ID NO: 130 - DNA, 12 bp, Artificial Sequence
  92a_LNA_12_2 miR-92 inhibitor
  misc_feature (1)..(12): May be joined by phosphorothioate bonds
  modified_base (1)..(1): May be a locked nucleic acid guanosine
  modified_base (2)..(2): May be a locked nucleic acid adenosine
  modified_base (4)..(4): May be a locked nucleic acid adenosine
  modified_base (6)..(6): May be a locked nucleic acid guanosine
  modified_base (7)..(7): May be a locked nucleic acid thymidine
  modified_base (9)..(9): May be a locked nucleic acid cytidine
  modified_base (11)..(11): May be a locked nucleic acid adenosine
  modified_base (12)..(12): May be a locked nucleic acid thymidine
gacaagtgcaat

SEQ ID NO: 131 - DNA, 12 bp, Artificial Sequence
  92a_LNA_12_3 miR-92 inhibitor
  misc_feature (1)..(12): May be joined by phosphorothioate bonds
  modified_base (1)..(1): May be a locked nucleic acid guanosine
  modified_base (3)..(3): May be a locked nucleic acid cytidine
  modified_base (4)..(4): May be a locked nucleic acid adenosine
  modified_base (6)..(6): May be a locked nucleic acid guanosine
  modified_base (8)..(8): May be a locked nucleic acid guanosine
  modified_base (10)..(10): May be a locked nucleic acid adenosine
  modified_base (12)..(12): May be a locked nucleic acid thymidine
gacaagtgcaat

SEQ ID NO: 132 - DNA, 12 bp, Artificial Sequence
  92a_LNA_12_4 miR-92 inhibitor
  misc_feature (1)..(12): May be joined by phosphorothioate bonds
  modified_base (1)..(1): May be a locked nucleic acid guanosine
  modified_base (3)..(3): May be a locked nucleic acid cytidine
  modified_base (5)..(5): May be a locked nucleic acid adenosine
  modified_base (6)..(6): May be a locked nucleic acid guanosine
  modified_base (8)..(8): May be a locked nucleic acid guanosine
  modified_base (10)..(10): May be a locked nucleic acid adenosine
  modified_base (12)..(12): May be a locked nucleic acid thymidine
gacaagtgcaat

SEQ ID NO: 133 - DNA, 12 bp, Artificial Sequence
  92a_LNA_12_5 miR-92 inhibitor
  misc_feature (1)..(12): May be joined by phosphorothioate bonds
  modified_base (1)..(1): May be a locked nucleic acid guanosine
  modified_base (2)..(2): May be a locked nucleic acid adenosine
  modified_base (5)..(5): May be a locked nucleic acid adenosine
  modified_base (6)..(6): May be a locked nucleic acid guanosine
  modified_base (7)..(7): May be a locked nucleic acid thymidine
  modified_base (9)..(9): May be a locked nucleic acid cytidine
  modified_base (10)..(10): May be a locked nucleic acid adenosine
  modified_base (12)..(12): May be a locked nucleic acid thymidine
gacaagtgcaat

SEQ ID NO: 134 - DNA, 12 bp, Artificial Sequence
  92a_LNA_12_6 miR-92 inhibitor
  misc_feature (1)..(12): May be joined by phosphorothioate bonds
  modified_base (1)..(1): May be a locked nucleic acid guanosine
  modified_base (3)..(3): May be a locked nucleic acid cytidine
  modified_base (5)..(5): May be a locked nucleic acid adenosine
  modified_base (7)..(7): May be a locked nucleic acid thymidine
  modified_base (9)..(9): May be a locked nucleic acid cytidine
  modified_base (12)..(12): May be a locked nucleic acid thymidine
gacaagtgcaat

SEQ ID NO: 135 - DNA, 12 bp, Artificial Sequence
  92a_LNA_12_7 miR-92 inhibitor
  misc_feature (1)..(12): May be joined by phosphorothioate bonds
  modified_base (1)..(1): May be a locked nucleic acid guanosine
  modified_base (2)..(2): May be a locked nucleic acid adenosine
  modified_base (4)..(4): May be a locked nucleic acid adenosine
  modified_base (6)..(6): May be a locked nucleic acid guanosine
  modified_base (8)..(8): May be a locked nucleic acid guanosine
  modified_base (10)..(10): May be a locked nucleic acid adenosine
  modified_base (12)..(12): May be a locked nucleic acid thymidine
gacaagtgcaat

SEQ ID NO: 136 - DNA, 12 bp, Artificial Sequence
  92a_LNA_12_8 miR-92 inhibitor
  misc_feature (1)..(12): May be joined by phosphorothioate bonds
  modified_base (1)..(1): May be a locked nucleic acid guanosine
  modified_base (4)..(4): May be a locked nucleic acid adenosine
  modified_base (8)..(8): May be a locked nucleic acid guanosine
  modified_base (9)..(9): May be a locked nucleic acid cytidine
  modified_base (10)..(11): May be a locked nucleic acid adenosine
  modified_base (12)..(12): May be a locked nucleic acid thymidine
gacaagtgcaat

SEQ ID NO: 137 - DNA, 12 bp, Artificial Sequence
  92a_LNA_12_9 miR-92 inhibitor
  misc_feature (1)..(12): May be joined by phosphorothioate bonds
  modified_base (1)..(1): May be a locked nucleic acid guanosine
  modified_base (2)..(2): May be a locked nucleic acid adenosine
  modified_base (4)..(4): May be a locked nucleic acid adenosine
  modified_base (6)..(6): May be a locked nucleic acid guanosine
  modified_base (10)..(11): May be a locked nucleic acid adenosine
  modified_base (12)..(12): May be a locked nucleic acid thymidine
gacaagtgcaat

SEQ ID NO: 138 - DNA, 12 bp, Artificial Sequence
  92a_LNA_12_10 miR-92 inhibitor
  misc_feature (1)..(12): May be joined by phosphorothioate bonds
  modified_base (1)..(1): May be a locked nucleic acid guanosine
  modified_base (2)..(2): May be a locked nucleic acid adenosine
  modified_base (4)..(4): May be a locked nucleic acid adenosine
  modified_base (7)..(7): May be a locked nucleic acid thymidine
  modified_base (10)..(11): May be a locked nucleic acid adenosine
  modified_base (12)..(12): May be a locked nucleic acid thymidine
gacaagtgcaat

SEQ ID NO: 139 - DNA, 12 bp, Artificial Sequence
  92a_LNA_12_11 miR-92 inhibitor
  misc_feature (1)..(12): May be joined by phosphorothioate bonds
  modified_base (1)..(1): May be a locked nucleic acid guanosine
  modified_base (4)..(4): May be a locked nucleic acid adenosine
  modified_base (7)..(7): May be a locked nucleic acid thymidine
  modified_base (10)..(11): May be a locked nucleic acid adenosine
  modified_base (12)..(12): May be a locked nucleic acid thymidine
gacaagtgcaat

SEQ ID NO: 140 - DNA, 12 bp, Artificial Sequence
  92a_LNA_12_12 miR-92 inhibitor
  misc_feature (1)..(12): May be joined by phosphorothioate bonds
  modified_base (1)..(1): May be a locked nucleic acid guanosine
  modified_base (2)..(2): May be a locked nucleic acid adenosine
  modified_base (4)..(4): May be a locked nucleic acid adenosine
  modified_base (7)..(7): May be a locked nucleic acid thymidine
  modified_base (9)..(9): May be a locked nucleic acid cytidine
  modified_base (10)..(10): May be a locked nucleic acid adenosine
  modified_base (12)..(12): May be a locked nucleic acid thymidine
gacaagtgcaat

SEQ ID NO: 141 - DNA, 12 bp, Artificial Sequence
  92a_LNA_12_13 miR-92 inhibitor
  misc_feature (1)..(12): May be joined by phosphorothioate bonds
  modified_base (1)..(1): May be a locked nucleic acid guanosine
  modified_base (3)..(3): May be a locked nucleic acid cytidine
  modified_base (6)..(6): May be a locked nucleic acid guanosine
  modified_base (9)..(9): May be a locked nucleic acid cytidine
  modified_base (11)..(11): May be a locked nucleic acid adenosine
  modified_base (12)..(12): May be a locked nucleic acid thymidine
gacaagtgcaat

SEQ ID NO: 142 - DNA, 12 bp, Artificial Sequence
  92a_LNA_12_14 miR-92 inhibitor
  misc_feature (1)..(12): May be joined by phosphorothioate bonds
  modified_base (1)..(1): May be a locked nucleic acid guanosine
  modified_base (3)..(3): May be a locked nucleic acid cytidine
  modified_base (5)..(5): May be a locked nucleic acid adenosine
  modified_base (6)..(6): May be a locked nucleic acid guanosine
  modified_base (8)..(8): May be a locked nucleic acid guanosine
  modified_base (9)..(9): May be a locked nucleic acid cytidine
  modified_base (12)..(12): May be a locked nucleic acid thymidine
gacaagtgcaat

SEQ ID NO: 143 - DNA, 12 bp, Artificial Sequence
  92a_LNA_12_15 miR-92 inhibitor
  misc_feature (1)..(12): May be joined by phosphorothioate bonds
  modified_base (1)..(1): May be a locked nucleic acid guanosine
  modified_base (2)..(2): May be a locked nucleic acid adenosine
  modified_base (5)..(5): May be a locked nucleic acid adenosine
  modified_base (7)..(7): May be a locked nucleic acid thymidine
  modified_base (9)..(9): May be a locked nucleic acid cytidine
  modified_base (10)..(10): May be a locked nucleic acid adenosine
  modified_base (12)..(12): May be a locked nucleic acid thymidine
gacaagtgcaat

SEQ ID NO: 144 - DNA, 12 bp, Artificial Sequence
  92a_LNA_12_16 miR-92 inhibitor
  misc_feature (1)..(12): May be joined by phosphorothioate bonds
  modified_base (1)..(1): May be a locked nucleic acid guanosine
  modified_base (2)..(2): May be a locked nucleic acid adenosine
  modified_base (4)..(4): May be a locked nucleic acid adenosine
  modified_base (6)..(6): May be a locked nucleic acid guanosine
  modified_base (8)..(8): May be a locked nucleic acid guanosine
  modified_base (10)..(10): May be a locked nucleic acid adenosine
  modified_base (12)..(12): May be a locked nucleic acid thymidine
gacaagtgcaat

SEQ ID NO: 145 - DNA, 12 bp, Artificial Sequence
  92a_LNA_12_17 miR-92 inhibitor
  misc_feature (1)..(12): May be joined by phosphorothioate bonds
  modified_base (1)..(1): May be a locked nucleic acid guanosine
  modified_base (3)..(3): May be a locked nucleic acid cytidine
  modified_base (6)..(6): May be a locked nucleic acid guanosine
  modified_base (7)..(7): May be a locked nucleic acid thymidine
  modified_base (10)..(11): May be a locked nucleic acid adenosine
  modified_base (12)..(12): May be a locked nucleic acid thymidine
gacaagtgcaat

SEQ ID NO: 146 - DNA, 12 bp, Artificial Sequence
  92a_LNA_12_18 miR-92 inhibitor
  misc_feature (1)..(12): May be joined by phosphorothioate bonds
  modified_base (1)..(1): May be a locked nucleic acid guanosine
  modified_base (2)..(2): May be a locked nucleic acid adenosine
  modified_base (4)..(4): May be a locked nucleic acid adenosine
  modified_base (7)..(7): May be a locked nucleic acid thymidine
  modified_base (9)..(9): May be a locked nucleic acid cytidine
  modified_base (11)..(11): May be a locked nucleic acid adenosine
  modified_base (12)..(12): May be a locked nucleic acid thymidine
gacaagtgcaat

SEQ ID NO: 147 - DNA, 12 bp, Artificial Sequence
  92a_LNA_12_19 miR-92 inhibitor
  misc_feature (1)..(12): May be joined by phosphorothioate bonds
  modified_base (1)..(1): May be a locked nucleic acid guanosine
  modified_base (4)..(4): May be a locked nucleic acid adenosine
  modified_base (6)..(6): May be a locked nucleic acid guanosine
  modified_base (9)..(9): May be a locked nucleic acid cytidine
  modified_base (10)..(10): May be a locked nucleic acid adenosine
  modified_base (12)..(12): May be a locked nucleic acid thymidine
gacaagtgcaat

SEQ ID NO: 148 - DNA, 12 bp, Artificial Sequence
  92a_LNA_12_20 miR-92 inhibitor
  misc_feature (1)..(12): May be joined by phosphorothioate bonds
  modified_base (1)..(1): May be a locked nucleic acid guanosine
  modified_base (3)..(3): May be a locked nucleic acid cytidine
  modified_base (4)..(4): May be a locked nucleic acid adenosine
  modified_base (6)..(6): May be a locked nucleic acid guanosine
  modified_base (9)..(9): May be a locked nucleic acid cytidine
  modified_base (11)..(11): May be a locked nucleic acid adenosine
  modified_base (12)..(12): May be a locked nucleic acid thymidine
gacaagtgcaat

SEQ ID NO: 149 - DNA, 12 bp, Artificial Sequence
  92a_LNA_12_21 miR-92 inhibitor
  misc_feature (1)..(12): May be joined by phosphorothioate bonds
  modified_base (1)..(1): May be a locked nucleic acid guanosine
  modified_base (3)..(3): May be a locked nucleic acid cytidine
  modified_base (5)..(5): May be a locked nucleic acid adenosine
  modified_base (6)..(6): May be a locked nucleic acid guanosine
  modified_base (8)..(8): May be a locked nucleic acid guanosine
  modified_base (10)..(10): May be a locked nucleic acid adenosine
  modified_base (12)..(12): May be a locked nucleic acid thymidine
gacaagtgcaat

SEQ ID NO: 150 - DNA, 12 bp, Artificial Sequence
  92a_LNA_12_22 miR-92 inhibitor
  misc_feature (1)..(12): May be joined by phosphorothioate bonds
  modified_base (1)..(1): May be a locked nucleic acid guanosine
  modified_base (2)..(2): May be a locked nucleic acid adenosine
  modified_base (3)..(3): May be a locked nucleic acid cytidine
  modified_base (6)..(6): May be a locked nucleic acid guanosine
  modified_base (9)..(9): May be a locked nucleic acid cytidine
  modified_base (11)..(11): May be a locked nucleic acid adenosine
  modified_base (12)..(12): May be a locked nucleic acid thymidine
gacaagtgcaat

SEQ ID NO: 151 - DNA, 12 bp, Artificial Sequence
  92a_LNA_12_23 miR-92 inhibitor
  misc_feature (1)..(12): May be joined by phosphorothioate bonds
  modified_base (1)..(1): May be a locked nucleic acid guanosine
  modified_base (3)..(3): May be a locked nucleic acid cytidine
  modified_base (6)..(6): May be a locked nucleic acid guanosine
  modified_base (8)..(8): May be a locked nucleic acid guanosine
  modified_base (9)..(9): May be a locked nucleic acid cytidine
  modified_base (10)..(10): May be a locked nucleic acid adenosine
  modified_base (12)..(12): May be a locked nucleic acid thymidine
gacaagtgcaat

SEQ ID NO: 152 - DNA, 12 bp, Artificial Sequence
  92a_LNA_12_24 miR-92 inhibitor
  misc_feature (1)..(12): May be joined by phosphorothioate bonds
  modified_base (1)..(1): May be a locked nucleic acid guanosine
  modified_base (3)..(3): May be a locked nucleic acid cytidine
  modified_base (4)..(4): May be a locked nucleic acid adenosine
  modified_base (6)..(6): May be a locked nucleic acid guanosine
  modified_base (8)..(8): May be a locked nucleic acid guanosine
  modified_base (10)..(10): May be a locked nucleic acid adenosine
  modified_base (12)..(12): May be a locked nucleic acid thymidine
gacaagtgcaat

SEQ ID NO: 153 - DNA, 16 bp, Artificial Sequence
  miR-92 inhibitor
  misc_feature (1)..(13): May be joined by phosphorothioate bonds
  modified_base (1)..(1): May be a locked nucleic acid cytidine
  modified_base (3)..(3): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 154 - DNA, 16 bp, Artificial Sequence
  miR-92 inhibitor
  misc_feature (1)..(12): May be joined by phosphorothioate bonds
  modified_base (1)..(1): May be a locked nucleic acid cytidine
  modified_base (3)..(3): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 155 - DNA, 16 bp, Artificial Sequence
  miR-92 inhibitor
  misc_feature (1)..(9): May be joined by phosphorothioate bonds
  modified_base (1)..(1): May be a locked nucleic acid cytidine
  modified_base (3)..(3): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 156 - DNA, 16 bp, Artificial Sequence
  miR-92 inhibitor
  misc_feature (1)..(2): May be joined by phosphorothioate bonds
  modified_base (1)..(1): May be a locked nucleic acid cytidine
  misc_feature (3)..(4): May be joined by phosphorothioate bonds
  modified_base (3)..(3): May be a locked nucleic acid guanosine
  misc_feature (5)..(6): May be joined by phosphorothioate bonds
  modified_base (6)..(6): May be a locked nucleic acid adenosine
  misc_feature (7)..(8): May be joined by phosphorothioate bonds
  modified_base (8)..(8): May be a locked nucleic acid adenosine
  misc_feature (9)..(10): May be joined by phosphorothioate bonds
  modified_base (10)..(10): May be a locked nucleic acid guanosine
  misc_feature (11)..(12): May be joined by phosphorothioate bonds
  modified_base (11)..(11): May be a locked nucleic acid thymidine
  misc_feature (13)..(14): May be joined by phosphorothioate bonds
  modified_base (13)..(13): May be a locked nucleic acid cytidine
  modified_base (14)..(14): May be a locked nucleic acid adenosine
  misc_feature (15)..(16): May be joined by phosphorothioate bonds
  modified_base (16)..(16): May be a locked nucleic acid thymidine
ccgggacaagtgcaat

SEQ ID NO: 157 - DNA, 16 bp, Artificial Sequence
  miR-92 inhibitor
  misc_feature (1)..(2): May be joined by phosphorothioate bonds
  modified_base (1)..(1): May be a locked nucleic acid cytidine
  modified_base (3)..(3): May be a locked nucleic acid guanosine
  misc_feature (4)..(5): May be joined by phosphorothioate bonds
  modified_base (6)..(6): May be a locked nucleic acid adenosine
  misc_feature (7)..(8): May be joined by phosphorothioate bonds
  modified_base (8)..(8): May be a locked nucleic acid adenosine
  misc_feature (10)..(11): May be joined by phosphorothioate bonds
  modified_base (10)..(10): May be a locked nucleic acid guanosine
  modified_base (11)..(11): May be a locked nucleic acid thymidine
  misc_feature (13)..(14): May be joined by phosphorothioate bonds
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 158 - DNA, 16 bp, Artificial Sequence
  miR-92 inhibitor
  modified_base (1)..(1): May be a locked nucleic acid cytidine
  modified_base (3)..(3): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 159 - DNA, 16 bp, Artificial Sequence
  miR-92 inhibitor
  misc_feature (1)..(13): May be joined by phosphorothioate bonds
  modified_base (1)..(1): May be a locked nucleic acid cytidine
  modified_base (2)..(2): May be a O-methyl deoxycytidine
  modified_base (3)..(3): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 160 - DNA, 16 bp, Artificial Sequence
  miR-92 inhibitor
  misc_feature (1)..(12): May be joined by phosphorothioate bonds
  modified_base (1)..(1): May be a locked nucleic acid cytidine
  modified_base (2)..(2): May be a O-methyl deoxycytidine
  modified_base (3)..(3): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 161 - DNA, 16 bp, Artificial Sequence
  miR-92 inhibitor
  misc_feature (1)..(9): May be joined by phosphorothioate bonds
  modified_base (1)..(1): May be a locked nucleic acid cytidine
  modified_base (2)..(2): May be a O-methyl deoxycytidine
  modified_base (3)..(3): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 162 - DNA, 16 bp, Artificial Sequence
  miR-92 inhibitor
  misc_feature (1)..(2): May be joined by phosphorothioate bonds
  modified_base (1)..(1): May be a locked nucleic acid cytidine
  modified_base (2)..(2): May be a O-methyl deoxycytidine
  misc_feature (3)..(4): May be joined by phosphorothioate bonds
  modified_base (3)..(3): May be a locked nucleic acid guanosine
  misc_feature (5)..(6): May be joined by phosphorothioate bonds
  modified_base (6)..(6): May be a locked nucleic acid adenosine
  misc_feature (7)..(8): May be joined by phosphorothioate bonds
  modified_base (8)..(8): May be a locked nucleic acid adenosine
  misc_feature (9)..(10): May be joined by phosphorothioate bonds
  modified_base (10)..(10): May be a locked nucleic acid guanosine
  misc_feature (11)..(12): May be joined by phosphorothioate bonds
  modified_base (11)..(11): May be a locked nucleic acid thymidine
  misc_feature (13)..(14): May be joined by phosphorothioate bonds
  modified_base (13)..(13): May be a locked nucleic acid cytidine
  modified_base (14)..(14): May be a locked nucleic acid adenosine
  misc_feature (15)..(16): May be joined by phosphorothioate bonds
  modified_base (16)..(16): May be a locked nucleic acid thymidine
ccgggacaagtgcaat

SEQ ID NO: 163 - DNA, 16 bp, Artificial Sequence
  miR-92 inhibitor
  misc_feature (1)..(2): May be joined by phosphorothioate bonds
  modified_base (1)..(1): May be a locked nucleic acid cytidine
  modified_base (2)..(2): May be a O-methyl deoxycytidine
  modified_base (3)..(3): May be a locked nucleic acid guanosine
  misc_feature (4)..(5): May be joined by phosphorothioate bonds
  modified_base (6)..(6): May be a locked nucleic acid adenosine
  misc_feature (7)..(8): May be joined by phosphorothioate bonds
  modified_base (8)..(8): May be a locked nucleic acid adenosine
  misc_feature (10)..(11): May be joined by phosphorothioate bonds
  modified_base (10)..(10): May be a locked nucleic acid guanosine
  modified_base (11)..(11): May be a locked nucleic acid thymidine
  misc_feature (13)..(14): May be joined by phosphorothioate bonds
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 164 - DNA, 16 bp, Artificial Sequence
  miR-92 inhibitor
  modified_base (1)..(1): May be a locked nucleic acid cytidine
  modified_base (2)..(2): May be a O-methyl deoxycytidine
  modified_base (3)..(3): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 165 - DNA, 16 bp, Artificial Sequence
  miR-92 inhibitor
  misc_feature (1)..(13): May be joined by phosphorothioate bonds
  modified_base (1)..(1): May be a locked nucleic acid cytidine
  modified_base (2)..(2): May be a O-methyl deoxycytidine
  modified_base (5)..(5): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (15)..(15): May be a locked nucleic acid adenosine
  modified_base (16)..(16): May be a locked nucleic acid thymidine
ccgggacaagtgcaat

SEQ ID NO: 166 - DNA, 16 bp, Artificial Sequence
  miR-92 inhibitor
  misc_feature (1)..(12): May be joined by phosphorothioate bonds
  modified_base (1)..(1): May be a locked nucleic acid cytidine
  modified_base (2)..(2): May be a O-methyl deoxycytidine
  modified_base (5)..(5): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (15)..(15): May be a locked nucleic acid adenosine
  modified_base (16)..(16): May be a locked nucleic acid thymidine
ccgggacaagtgcaat

SEQ ID NO: 167 - DNA, 16 bp, Artificial Sequence
  miR-92 inhibitor
  misc_feature (1)..(9): May be joined by phosphorothioate bonds
  modified_base (1)..(1): May be a locked nucleic acid cytidine
  modified_base (2)..(2): May be a O-methyl deoxycytidine
  modified_base (5)..(5): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (15)..(15): May be a locked nucleic acid adenosine
  modified_base (16)..(16): May be a locked nucleic acid thymidine
ccgggacaagtgcaat

SEQ ID NO: 168 - DNA, 16 bp, Artificial Sequence
  miR-92 inhibitor
  misc_feature (1)..(2): May be joined by phosphorothioate bonds
  modified_base (1)..(1): May be a locked nucleic acid cytidine
  modified_base (2)..(2): May be a O-methyl deoxycytidine
  misc_feature (3)..(4): May be joined by phosphorothioate bonds
  misc_feature (5)..(6): May be joined by phosphorothioate bonds
  modified_base (5)..(5): May be a locked nucleic acid guanosine
  modified_base (6)..(6): May be a locked nucleic acid adenosine
  misc_feature (7)..(8): May be joined by phosphorothioate bonds
  modified_base (8)..(8): May be a locked nucleic acid adenosine
  misc_feature (9)..(10): May be joined by phosphorothioate bonds
  modified_base (10)..(10): May be a locked nucleic acid guanosine
  misc_feature (11)..(12): May be joined by phosphorothioate bonds
  modified_base (11)..(11): May be a locked nucleic acid thymidine
  misc_feature (13)..(14): May be joined by phosphorothioate bonds
  modified_base (13)..(13): May be a locked nucleic acid cytidine
  misc_feature (15)..(16): May be joined by phosphorothioate bonds
  modified_base (15)..(15): May be a locked nucleic acid adenosine
  modified_base (16)..(16): May be a locked nucleic acid thymidine
ccgggacaagtgcaat

SEQ ID NO: 169 - DNA, 16 bp, Artificial Sequence
  miR-92 inhibitor
  misc_feature (1)..(2): May be joined by phosphorothioate bonds
  modified_base (1)..(1): May be a locked nucleic acid cytidine
  modified_base (2)..(2): May be a O-methyl deoxycytidine
  misc_feature (4)..(5): May be joined by phosphorothioate bonds
  modified_base (5)..(5): May be a locked nucleic acid guanosine
  modified_base (6)..(6): May be a locked nucleic acid adenosine
  misc_feature (7)..(8): May be joined by phosphorothioate bonds
  modified_base (8)..(8): May be a locked nucleic acid adenosine
  misc_feature (10)..(11): May be joined by phosphorothioate bonds
  modified_base (10)..(10): May be a locked nucleic acid guanosine
  modified_base (11)..(11): May be a locked nucleic acid thymidine
  misc_feature (13)..(14): May be joined by phosphorothioate bonds
  modified_base (13)..(13): May be a locked nucleic acid cytidine
  modified_base (15)..(15): May be a locked nucleic acid adenosine
  modified_base (16)..(16): May be a locked nucleic acid thymidine
ccgggacaagtgcaat

SEQ ID NO: 170 - DNA, 16 bp, Artificial Sequence
  miR-92 inhibitor
  modified_base (1)..(1): May be a locked nucleic acid cytidine
  modified_base (2)..(2): May be a O-methyl deoxycytidine
  modified_base (5)..(5): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (15)..(15): May be a locked nucleic acid adenosine
  modified_base (16)..(16): May be a locked nucleic acid thymidine
ccgggacaagtgcaat

SEQ ID NO: 171 - DNA, 16 bp, Artificial Sequence
  miR-92 inhibitor
  misc_feature (1)..(13): May be joined by phosphorothioate bonds
  modified_base (1)..(1): May be a locked nucleic acid cytidine
  modified_base (2)..(2): May be a O-methyl deoxycytidine
  modified_base (3)..(3): May be a locked nucleic acid guanosine
  modified_base (6)..(6): May be a locked nucleic acid adenosine
  modified_base (9)..(9): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 172 - DNA, 16 bp, Artificial Sequence
  miR-92 inhibitor
  misc_feature (1)..(12): May be joined by phosphorothioate bonds
  modified_base (1)..(1): May be a locked nucleic acid cytidine
  modified_base (2)..(2): May be a O-methyl deoxycytidine
  modified_base (3)..(3): May be a locked nucleic acid guanosine
  modified_base (6)..(6): May be a locked nucleic acid adenosine
  modified_base (9)..(9): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 173 - DNA, 16 bp, Artificial Sequence
  miR-92 inhibitor
  misc_feature (1)..(9): May be joined by phosphorothioate bonds
  modified_base (1)..(1): May be a locked nucleic acid cytidine
  modified_base (2)..(2): May be a O-methyl deoxycytidine
  modified_base (3)..(3): May be a locked nucleic acid guanosine
  modified_base (6)..(6): May be a locked nucleic acid adenosine
  modified_base (9)..(9): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 174 - DNA, 16 bp, Artificial Sequence
  miR-92 inhibitor
  misc_feature (1)..(2): May be joined by phosphorothioate bonds
  modified_base (1)..(1): May be a locked nucleic acid cytidine
  modified_base (2)..(2): May be a O-methyl deoxycytidine
  misc_feature (3)..(4): May be joined by phosphorothioate bonds
  modified_base (3)..(3): May be a locked nucleic acid guanosine
  misc_feature (5)..(6): May be joined by phosphorothioate bonds
  modified_base (6)..(6): May be a locked nucleic acid adenosine
  misc_feature (7)..(8): May be joined by phosphorothioate bonds
  misc_feature (9)..(10): May be joined by phosphorothioate bonds
  modified_base (9)..(9): May be a locked nucleic acid adenosine
  modified_base (10)..(10): May be a locked nucleic acid guanosine
  misc_feature (11)..(12): May be joined by phosphorothioate bonds
  modified_base (11)..(11): May be a locked nucleic acid thymidine
  misc_feature (13)..(14): May be joined by phosphorothioate bonds
  modified_base (13)..(13): May be a locked nucleic acid cytidine
  modified_base (14)..(14): May be a locked nucleic acid adenosine
  misc_feature (15)..(16): May be joined by phosphorothioate bonds
  modified_base (16)..(16): May be a locked nucleic acid thymidine
ccgggacaagtgcaat

SEQ ID NO: 175 - DNA, 16 bp, Artificial Sequence
  miR-92 inhibitor
  misc_feature (1)..(2): May be joined by phosphorothioate bonds
  modified_base (1)..(1): May be a locked nucleic acid cytidine
  modified_base (2)..(2): May be a O-methyl deoxycytidine
  modified_base (3)..(3): May be a locked nucleic acid guanosine
  misc_feature (4)..(5): May be joined by phosphorothioate bonds
  modified_base (6)..(6): May be a locked nucleic acid adenosine
  misc_feature (7)..(8): May be joined by phosphorothioate bonds
  modified_base (9)..(9): May be a locked nucleic acid adenosine
  misc_feature (10)..(11): May be joined by phosphorothioate bonds
  modified_base (10)..(10): May be a locked nucleic acid guanosine
  modified_base (11)..(11): May be a locked nucleic acid thymidine
  misc_feature (13)..(14): May be joined by phosphorothioate bonds
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 176 - DNA, 16 bp, Artificial Sequence
  miR-92 inhibitor
  modified_base (1)..(1): May be a locked nucleic acid cytidine
  modified_base (2)..(2): May be a O-methyl deoxycytidine
  modified_base (3)..(3): May be a locked nucleic acid guanosine
  modified_base (6)..(6): May be a locked nucleic acid adenosine
  modified_base (9)..(9): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 177 - DNA, 16 bp, Artificial Sequence
  miR-92 inhibitor
  misc_feature (1)..(16): May be joined by phosphorothioate bonds
  modified_base (1)..(1): May be a locked nucleic acid cytidine
  modified_base (5)..(5): May be a locked nucleic acid guanosine
  modified_base (6)..(6): May be a locked nucleic acid adenosine
  modified_base (9)..(9): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (15)..(15): May be a locked nucleic acid adenosine
  modified_base (16)..(16): May be a locked nucleic acid thymidine
ccgggacaagtgcaat

SEQ ID NO: 178 - DNA, 16 bp, Artificial Sequence
  miR-92 inhibitor
  misc_feature (1)..(16): May be joined by phosphorothioate bonds
  modified_base (1)..(1): May be a locked nucleic acid cytidine
  modified_base (5)..(5): May be a locked nucleic acid guanosine
  modified_base (6)..(6): May be a locked nucleic acid adenosine
  modified_base (8)..(8): May be a locked nucleic acid adenosine
  modified_base (10)..(10): May be a locked nucleic acid guanosine
  modified_base (11)..(11): May be a locked nucleic acid thymidine
  modified_base (13)..(13): May be a locked nucleic acid cytidine
  modified_base (14)..(14): May be a locked nucleic acid adenosine
  modified_base (16)..(16): May be a locked nucleic acid thymidine
ccgggacaagtgcaat

SEQ ID NO: 179 - DNA, 16 bp, Artificial Sequence
  miR-92 inhibitor
  misc_feature (1)..(16): May be joined by phosphorothioate bonds
  modified_base (1)..(1): May be a locked nucleic acid cytidine
  modified_base (4)..(4): May be a locked nucleic acid guanosine
  modified_base (5)..(5): May be a locked nucleic acid guanosine
  modified_base (7)..(7): May be a locked nucleic acid cytidine
  modified_base (9)..(9): May be a locked nucleic acid adenosine
  modified_base (10)..(10): May be a locked nucleic acid guanosine
  modified_base (12)..(12): May be a locked nucleic acid guanosine
  modified_base (14)..(14): May be a locked nucleic acid adenosine
  modified_base (16)..(16): May be a locked nucleic acid thymidine
ccgggacaagtgcaat

SEQ ID NO: 180 - RNA, 23 nt, Artificial Sequence
  miR-19b mimic
  modified_base (1)..(1): May be a O-methyl uridine
  modified_base (2)..(2): May be a O-methyl cytidine
  modified_base (5)..(7): May be a O-methyl uridine
  modified_base (10)..(10): May be a O-methyl cytidine
  modified_base (11)..(12): May be a O-methyl uridine
  modified_base (16)..(18): May be a O-methyl uridine
  modified_base (22)..(22): May be a O-methyl cytidine
  misc_feature (23)..(23): May have a  cholesterol conjugate with a 6 carbon linker attached
ucaguuuagcuuggauuuggaca

SEQ ID NO: 181 - RNA, 25 nt, Artificial Sequence
  miR-19b mimic
  modified_base (3)..(3): May be a fluoro uridine
  modified_base (5)..(5): May be a fluoro cytidine
  modified_base (9)..(9): May be a fluoro uridine
  modified_base (10)..(11): May be a fluoro cytidine
  modified_base (13)..(13): May be a fluoro uridine
  modified_base (15)..(15): May be a fluoro cytidine
  modified_base (20)..(20): May be a fluoro cytidine
  modified_base (21)..(21): May be a fluoro uridine
  misc_feature (23)..(25): May be joined by phosphorothioate bonds
ugugcaaauccaugcaaaacugauu

SEQ ID NO: 182 - DNA, 22 bp, Artificial Sequence
  mGAPDH primer
aatgtgtccgtcgtggatctga

SEQ ID NO: 183 - DNA, 22 bp, Artificial Sequence
  mGAPDH primer
agtgtagcccaagatgcccttc

SEQ ID NO: 184 - DNA, 20 bp, Artificial Sequence
  mFZD4 primer
agagagaagagggggaatgg

SEQ ID NO: 185 - DNA, 20 bp, Artificial Sequence
  mFZD4 primer
tgtgtgtgggctgaagtgtt

SEQ ID NO: 186 - DNA, 20 bp, Artificial Sequence
  mLrp6 primer
tgtggtaaaccccgagaaag

SEQ ID NO: 187 - DNA, 19 bp, Artificial Sequence
  mLrp6 primer
atcctgttggcacctgaga

SEQ ID NO: 188 - DNA, 30 bp, Homo sapiens
ataggauuuuuuuuuuuugcacugcctgca

SEQ ID NO: 189 - DNA, 30 bp, Homo sapiens
aaauuucuuggcaacuuugcauucacacag

SEQ ID NO: 190 - DNA, 30 bp, Homo sapiens
acuaaaaguuuuauuuuugcaaacuaaaua

(19) United States
(12) Patent Application Publication
(10) Pub. No.: US 2018/0250325 A1
(43) Pub. Date: Sep. 6, 2018

(54) MIR-19 MODULATORS AND USES THEREOF

(71) Applicant: MIRAGEN THERAPEUTICS, INC., Boulder, CO (US)

(72) Inventors: William C. SESSA, Madison, CT (US); Christina Marie DALBY, Boulder, CO (US); Corrie Lynn GALLANT-BEHM, Erie, CO (US)

(21) Appl. No.: 15/762,508

(22) PCT Filed: Sep. 22, 2016
(86) PCT No.: PCT/US16/53192

§ 371 Date: Mar. 22, 2018

Related U.S. Application Data
(60) Provisional application No. 62/222,079, filed on Sep. 22, 2015.

(51) Int. Cl. A61K 31/7105; C12N 15/113; G01N 33/50

(52) U.S. Cl. CPC A61K 31/7105; C12N 15/113; A61P 17/02; C12N 2310/113; C12N 2320/31; G01N 33/5008

(57) ABSTRACT

The present invention provides miR-19 modulators and uses thereof, such as for promoting angiogenesis and/or wound healing with miR-19 inhibitors alone or in combination with other agents. The present invention also provides methods of treating or preventing arterial and cardiac conditions with a miR-19 inhibitor. Also provided are oligonucleotides with chemical motifs that are miR-19 inhibitors, and methods of using the oligonucleotides for inhibiting the function or activity of miR-19 in a subject in need thereof.

### CROSS REFERENCE TO RELATED APPLICATIONS

The present application claims the benefit of priority to U.S. Provisional Application No. 62/222,079, filed on Sep. 22, 2015, the contents of which are hereby incorporated by reference in their entirety.

### STATEMENT AS TO FEDERALLY SPONSORED RESEARCH

This invention was made with U.S. government support under grant number HL096670 awarded by the National Institutes of Health. The U.S. government may have certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is MIRG_048_02WO_SeqList_ST25.txt. The text file is 174 KB, was created on Sep. 22, 2016, and is being submitted electronically via EFS-Web.

### FIELD OF THE INVENTION

The present invention relates generally to modulators of miR-19 function and/or activity, for example, oligonucleotides with chemical motifs that are miR-19 inhibitors, and uses thereof.

### BACKGROUND OF THE INVENTION

MicroRNAs (miRNAs) are a class of small, endogenous and non-coding RNAs able to negatively regulate gene expression by targeting specific messenger RNAs (mRNAs) and inducing their degradation or translational repression (Ambros, Nature 431:350-355 (2004); Bartel, Cell 136:215-233 (2009)). A recent study has defined mRNA degradation as the predominant mechanistic effect of miRNA:mRNA targets (Guo et al., Nature 2010; 466:835-840).

MicroRNAs have been implicated in a number of biological processes including regulation and maintenance of cardiac function, vascular inflammation and development of vascular pathologies (see Eva Van Rooij and Eric Olson, J. Clin. Invest. 117(9):2369-2376 (2007); Chien, Nature 447:389-390 (2007); Kartha and Subramanian, J. Cardiovasc. Transl. Res. 3:256-270 (2010); Urbich et al., Cardiovasc. Res. 79:581-588 (2008)). MiRNAs have also been reported to be involved in the development of organisms (Ambros, Cell 113:673-676 (2003)) and are differentially expressed in numerous tissues (Xu et al., Curr. Biol. 13:790-795 (2003); Landgraf et al., Cell 129:1401-14 (2007)), in viral infection processes (Pfeffer et al., Science 304:734-736 (2004)), and associated with oncogenesis (Calin et al., Proc. Natl. Acad. Sci. USA 101:2999-3004 (2004)); Calin et al., Proc. Natl. Acad. Sci. USA 99(24):15524-15529 (2002)).

Accordingly, modulating the function and/or activity of microRNAs present therapeutic targets in the development of effective treatments for a variety of conditions. However, delivery of an antisense-based therapeutic targeting a miRNA can pose several challenges. The binding affinity and specificity to a specific miRNA, efficiency of cellular uptake, and nuclease resistance are all factors in the delivery and activity of an oligonucleotide-based therapeutic. For example, when oligonucleotides are introduced into intact cells they are typically attacked and degraded by nucleases leading to a loss of activity. Thus, a useful antisense therapeutic should have good resistance to extra- and intracellular nucleases, as well as be able to penetrate the cell membrane.

Accordingly, there is a need for identifying miRNAs associated with disease and methods of treating diseases, injuries and/or conditions by modulating the activity of miRNAs associated with disease. The present invention meets these needs and provides related advantages as well.

The oligonucleotides provided herein can have advantages in potency, efficiency of delivery, target specificity, stability, and/or toxicity when administered to a subject.

### SUMMARY OF THE INVENTION

In one aspect, provided herein is a method for promoting wound healing in a subject in need thereof, comprising administering an oligonucleotide inhibitor of miR-19 comprising a sequence complementary to miR-19. In one embodiment, the administration of the oligonucleotide inhibitor of miR-19 reduces function or activity of miR-19. In one embodiment, the oligonucleotide inhibitor of miR-19 is selected from Table 1. In one embodiment, the method further comprises administering an additional agent for promoting wound healing. In one embodiment, the additional agent is an oligonucleotide inhibitor of miR-92 comprising a sequence complementary to miR-92. In one embodiment, the administration of the oligonucleotide inhibitor of miR-92 reduces function or activity of miR-92. In one embodiment, the oligonucleotide inhibitor of miR-92 is selected from Table 2. In one embodiment, the oligonucleotide inhibitor of miR-19 and the additional agent are administered sequentially. In one embodiment, the oligonucleotide inhibitor of miR-19 and the additional agent are administered simultaneously. In one embodiment, the method further comprises adding a growth factor. In one embodiment, the growth factor is platelet derived growth factor (PDGF) and/or vascular endothelial growth factor (VEGF). In one embodiment, the subject is human. In one embodiment, the subject suffers from diabetes. In one embodiment, the wound healing is for a chronic wound, diabetic foot ulcer, venous stasis leg ulcer or pressure sore. In one embodiment, the administration of the oligonucleotide inhibitor of miR-19 produces an increased rate of re-epithelialization, granulation, and/or neoangiogenesis during wound healing as compared to no treatment. In one embodiment, the administration of the oligonucleotide inhibitor of miR-19 and the oligonucleotide inhibitor of miR-92 produces an increased rate of re-epithelialization, granulation, and/or neoangiogenesis during wound healing as compared to no treatment or treatment with either the oligonucleotide inhibitor of miR-19 or the oligonucleotide inhibitor of miR-92 alone.

In a further aspect, provided herein is an oligonucleotide inhibitor comprising a sequence complementary to miR-19, wherein the sequence further comprises one or more locked nucleic acid (LNA) nucleotides and one or more non-locked nucleotides, wherein at least one of the non-locked nucleotides comprises a chemical modification. In one embodiment, the oligonucleotide inhibitor is complementary to miR-19a. In one embodiment, the oligonucleotide inhibitor is complementary to miR-19b. In one embodiment, the locked nucleic acid (LNA) nucleotide has a 2′ to 4′ methylene bridge. In one embodiment, the chemical modification is a 2′ O-alkyl or 2′ halo modification. In one embodiment, the oligonucleotide inhibitor has a 5′ cap structure, 3′ cap structure, or 5′ and 3′ cap structure. In one embodiment, the oligonucleotide inhibitor further comprises a pendent lipophilic group. In one embodiment, the sequence is selected from Table 1.

In a further aspect, provided herein is a pharmaceutical composition comprising an oligonucleotide inhibitor comprising a sequence complementary to miR-19, wherein the sequence further comprises one or more locked nucleic acid (LNA) nucleotides and one or more non-locked nucleotides, wherein at least one of the non-locked nucleotides comprises a chemical modification, or a pharmaceutically-acceptable salt thereof, and a pharmaceutically-acceptable carrier or diluent. In one embodiment, the oligonucleotide inhibitor is complementary to miR-19a. In one embodiment, the oligonucleotide inhibitor is complementary to miR-19b. In one embodiment, the locked nucleic acid (LNA) nucleotide has a 2′ to 4′ methylene bridge. In one embodiment, the chemical modification is a 2′ O-alkyl or 2′ halo modification. In one embodiment, the oligonucleotide inhibitor has a 5′ cap structure, 3′ cap structure, or 5′ and 3′ cap structure. In one embodiment, the oligonucleotide inhibitor further comprises a pendent lipophilic group. In one embodiment, the sequence is selected from Table 1. In one embodiment, the pharmaceutical composition further comprises an oligonucleotide inhibitor of miR-92 comprising a sequence complementary to miR-92. In one embodiment, the sequence is selected from Table 2. In one embodiment, a molar ratio of an amount of the oligonucleotide inhibitor of miR-19 to an amount of the oligonucleotide inhibitor of miR-92 in the composition is from about 1:99 to about 99:1. In one embodiment, the molar ratio of the oligonucleotide inhibitor of miR-19 to the oligonucleotide inhibitor of miR-92 is about 1:1. In one embodiment, the pharmaceutical composition is used in a method of treating a wound in a subject in need thereof, comprising administering the pharmaceutical composition to the subject. In one embodiment, the wound is a chronic wound, diabetic foot ulcer, venous stasis leg ulcer or pressure sore.

In yet another aspect, provided herein is a method for evaluating or monitoring the efficacy of a therapeutic for modulating wound healing in a subject receiving the therapeutic comprising: a.) measuring the expression of one or more genes that are targets of miR-19 from a sample from a subject; and b.) comparing the expression of the one or more genes that are targets of miR-19 to a pre-determined reference level or level of the one or more genes that are targets of miR-19 in a control sample, wherein the comparison is indicative of the efficacy of the therapeutic, wherein the therapeutic is an oligonucleotide comprising a sequence selected from Table 1. In one embodiment, the one or more genes that are targets of miR-19 are frizzled-4 (FZD4) or low-density lipoprotein receptor-related protein 6 (LRP6). In one embodiment, the therapeutic modulates miR-19 function and/or activity. In one embodiment, the subject suffers from ischemia, myocardial infarction, chronic ischemic heart disease, peripheral or coronary artery occlusion, ischemic infarction, stroke, atherosclerosis, acute coronary syndrome, coronary artery disease, carotid artery disease, diabetes, chronic wound(s), peripheral vascular disease or peripheral artery disease. In one embodiment, the subject is a human. In another aspect, provided herein is a method for evaluating an agent's ability to promote angiogenesis or wound healing comprising: a.) contacting a cell with the agent, wherein the agent is an oligonucleotide inhibitor comprising a sequence selected from Table 1; b.) measuring the expression of one or more genes that are targets of miR-19 in the cell contacted with the agent; and c.) comparing the expression of the one or more genes that are targets of miR-19 to a pre-determined reference level or level of the one or more genes that are targets of miR-19 in a control sample, wherein the comparison is indicative of the agent's ability to promote angiogenesis or wound healing. In one embodiment, the one or more genes that are targets of miR-19 are FZD4 or LRP6. In one embodiment, the method further comprises determining miR-19 function and/or activity in the cell contacted with the agent. In one embodiment, the cell is a mammalian cell. In one embodiment, the cell is a cardiac cell, muscle cell, fibrocyte, fibroblast, keratinocyte or endothelial cell. In one embodiment, the cell is in vitro, in vivo or ex vivo.

### BRIEF DESCRIPTION OF THE DRAWINGS

FIG. 1A illustrates perfusion quantified in mice injected daily subcutaneously with control or antimiR-19 at a dose of 12.5 mg/kg for 3 days prior to surgery then weekly thereafter by measuring gastrochnemius flow pre- and post-surgery, followed by weekly measurements using a deep penetrating laser doppler probe. Data in FIG. 1A is n=9 mice per group, *p<0.05, two way ANOVA. FIG. 1B illustrates antimiR-19, but not control, increased reporter gene expression in capillary EC surrounding regenerating muscle fibers in ischemic tissue using an LNA-antimiR approach and HLI in BAT gal mice.

FIG. 2A-C illustrates that treatment with antimiR-19 reduced miR-19 levels (FIG. 2A) and upregulated the mRNA levels of direct targets of miR-19, FRZD4 (FIG. 2B) and LRP6 (FIG. 2C) in the tissue of mice that were administered antimiR-19 as described in Example 1. Data are from n=4 mice per group; *p<0.05, two way ANOVA. All data are mean+/−SEM.

FIG. 3A illustrates a schematic representation of the sequences of miR-19 (SEQ ID NO: 1) predicted binding sites in the 3′UTR of FZD4 (SEQ ID NOs: 188 and 189) and LRP6 (SEQ ID NO: 190). Mutation sites are marked with an asterisk (*). Sequence difference between miR19a and miR19b is marked with _. FIG. 3B illustrates that the mutation (* in FIG. 3A) of predicted miR-19 target sites reduced miR-19 mediated repression of 3′UTR LUC activity of FZD4 and LRP6 in a luciferase assay. Data are mean+/−SEM from 3 independent experiments.

FIG. 4A illustrates results from Mouse lung endothelial cells (MLECs) transfected with either miR-19 mimic or mimic control. Following 48 hrs, cells were treated with Wnt Family Member 3A (WNT3a). miR-19 transfected cells resulted in reduced expression of several (3-catenin dependent genes in response to WNT3a treatment-including Axin2, Sox17 and Cyclin D1. FIG. 4B illustrates results from MLECs transfected with control or anti-miR-19 (60 nM of each) for 48 hours prior to WNT3a stimulation. Cells were starved for 4 hours then treated with WNT3a conditioned media for time points above. Lysates were collected and run on SDS-PAGE gel and immunoblotted for p-JNK, total JNK, and Hsp90.

FIG. 5A-D illustrates cutaneous wound healing parameters in diabetic mice injected intradermally with control, antimiR-92 (30 nmol and 60 nmol doses), antimiR-19 (30 nmol and 60 nmol doses) or a combination of antimiR-92 and antimiR-19 (30 nmol of each) at the site of a skin wound. FIG. 5A illustrates the percent re-epithelialization ({1−[epithelial gap divided by wound width]}×100), FIG. 5B illustrates the percent of each wound filled that was filled with granulation tissue ({1−[granulation tissue gap divided by wound width]}×100), FIG. 5C illustrates the granulation tissue area within the wound, and FIG. 5D illustrates the average thickness of granulation tissue within the wound. Data are from n=10 mice per group, 2 wounds per mouse; *p>0.05, **p<0.01, ***p<0.001, ****p<0.0001, Kruskal-Wallis test with Dunn's multiple comparison's test. All data are mean+/−SEM.

### DETAILED DESCRIPTION OF THE INVENTION

MiR-19 is located in the miR-17-92 cluster, which consists of miR-17-5p, miR-17-3p, miR-18a, miR-19a, miR-20a, miR-19b, and miR-92-1 (Venturini et al., Blood 109 10:4399-4405 (2007)). The pre-miRNA sequence for miR-19 is processed into a mature sequence (3p) and a star (i.e. minor or 5p) sequence. The star sequence is processed from the other arm of the stem loop structure. The mature and star miRNA sequences for human and mouse miR-19 are provided:

Human mature miR-19a (i.e. hsa-miR-19a-3p)
(SEQ ID NO: 1)
5′-UGUGCAAAUCUAUGCAAAACUGA-3′
Human miR-19a* (i.e. hsa-miR-19a-5p)
(SEQ ID NO: 2)
5′-AGUUUUGCAUAGUUGCACUACA-3′
Human mature miR-19b (i.e. hsa-miR-19b-3p)
(SEQ ID NO: 3)
5′-UGUGCAAAUCCAUGCAAAACUGA-3′
Human miR-19b-1* (i.e. hsa-miR-19b-1-5p)
(SEQ ID NO: 4)
5′-AGUUUUGCAGGUUUGCAUCCAGC-3′
Human miR-19b-2* (i.e. hsa-miR-19b-2-5p)
(SEQ ID NO: 5)
5′-AGUUUUGCAGGUUUGCAUUUCA-3′
Mouse mature miR-19a (i.e. mmu-miR-19a-3p)
(SEQ ID NO: 6)
5′-UGUGCAAAUCUAUGCAAAACUGA-3′
Mouse miR-19a* (i.e. mmu-miR-19a-5p)
(SEQ ID NO: 7)
5′-UAGUUUUGCAUAGUUGCACUAC-3′
Mouse mature miR-19b (i.e. mmu-miR-19b-3p)
(SEQ ID NO: 8)
5′-UGUGCAAAUCCAUGCAAAACUGA-3′
Mouse miR-19b-1* (i.e. mmu-miR-19b-1-5p)
(SEQ ID NO: 9)
5′-AGUUUUGCAGGUUUGCAUCCAGC-3′
Mouse miR-19b-2* (i.e. mmu-miR-19b-2-5p)
(SEQ ID NO: 10)
5′-AGUUUUGCAGAUUUGCAGUUCAGC-3′

The present invention provides oligonucleotide inhibitors that reduce or inhibit the activity or function of miR-19 (e.g., human miR-19) and compositions and uses thereof. Also provided herein are miR-19 agonists, such as a miR-19 mimic.

The term “miR-19” as used herein includes pri-miR-19, pre-miR-19, miR-19, miR-19a, miR-19b, miR-19a-3p, miR-19b-3p, hsa-miR-19a-3p and hsa-miR-19b-3p.

In one embodiment, the oligonucleotide inhibitor of miR-19 is an inhibitor of a miR-19 as described herein (e.g., miR-19a, miR-19b, miR-19a*, miR-19b-1*, miR-19b-2*). In another embodiment, the oligonucleotide inhibitor of miR-19 is an inhibitor of miR-19a, miR-19b, or both miR-19a and miR-19b. In yet another embodiment, the miR-19 inhibitor is a miR-19b inhibitor. In a further embodiment, the miR-19 inhibitor is a miR-19a inhibitor.

The sequence of an oligonucleotide inhibitor of miR-19 according to the present invention is sufficiently complementary to a sequence of miR-19 as to hybridize to miR-19 under physiological conditions and inhibit the activity or function of miR-19 in a cell or cells of a subject. For example, in some embodiments, the oligonucleotide inhibitor can consist of, consist essentially of or comprise a sequence that is at least partially complementary to a mature miR-19 (e.g., miR-19a or miR-19b) sequence, e.g. at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% complementary to a mature sequence of miR-19 (e.g., miR-19a or miR-19b). In one embodiment, the oligonucleotide inhibitor (also referred to as antisense oligonucleotide) consists of, consists essentially of or comprises a sequence that is 100% complementary to a mature miR-19 (e.g., miR-19a or miR-19b) sequence. In this context, “consists essentially of” includes the optional addition of nucleotides (e.g., one or two) on either or both of the 5′ and 3′ ends, so long as the additional nucleotide(s) do not substantially affect (as defined by an increase in IC50 of no more than 20%) the oligonucleotide's inhibition of miR-19 activity in a cell in a subject or an assay as provided herein. It is understood that the sequence of the oligonucleotide inhibitor is considered to be complementary to miR-19 even if the oligonucleotide inhibitor sequence includes a modified nucleotide instead of a naturally-occurring nucleotide. For example, if a mature sequence of miR-19 comprises a guanosine nucleotide at a specific position, the oligonucleotide inhibitor may comprise a modified cytidine nucleotide, such as a locked cytidine nucleotide or 2′-fluoro-cytidine, at the corresponding position. In certain embodiments, the oligonucleotide inhibitor may be designed to have a sequence containing from 1 to 5 (e.g., 1, 2, 3, or 4) mismatches relative to the fully complementary (mature) miR-19 (e.g., miR-19a or miR-19b) sequence. In certain embodiments, such antisense sequences may be incorporated into shRNAs or other RNA structures containing stem and loop portions, for example.

In some embodiments, the entire sequence of the oligonucleotide inhibitor of miR-19 is fully complementary to a mature sequence of human miR-19b-3p. In various embodiments, the mature sequence of human miR-19b-3p to which the sequence of the oligonucleotide inhibitor of the present invention is partially, substantially, or fully complementary to includes nucleotides 1-23 or nucleotides 2-15 from the 5′ end of SEQ ID NO: 3. In one embodiment, the mature sequence of human miR-19b-3p to which the sequence of the oligonucleotide inhibitor of the present invention is partially, substantially, or fully complementary to includes nucleotides 2-15 from the 5′ end of SEQ ID NO: 3.

In one embodiment, a oligonucleotide inhibitor of miR-19 as provided herein is administered with an inhibitor of another miRNA. Both inhibitors can be present in a single composition (e.g., pharmaceutical composition as provided herein) or in separate compositions (e.g., pharmaceutical compositions as provided herein). In one embodiment, the miR-19 inhibitor is administered with an inhibitor of an miRNA located in the miR-17-92 cluster. In one embodiment, the miR-19 inhibitor is administered with an oligonucleotide inhibitor of miR-92, such as, for example, a miR-92 inhibitor disclosed in US20160208258, the contents of which are herein incorporated by reference in their entirety for all purposes.

Accordingly, the present invention also provides oligonucleotide inhibitors that reduce or inhibit the activity or function of miR-92.

The term “miR-92” as used herein includes pri-miR-92, pre-miR-92, miR-92, miR-92a, miR-92b, miR-92a-3p, and hsa-miR-92a-3p.

The mature and star miRNA sequences for human, mouse, and rat miR-92 are provided:

Human mature miR-92 (i.e. hsa-miR-92a-3p)
(SEQ ID NO: 13)
5′-UAUUGCACUUGUCCCGGCCUGU-3′
Human miR-92a-1* (i.e. hsa-miR-92a-1-5p)
(SEQ ID NO: 14)
5′-AGGUUGGGAUCGGUUGCAAUGCU-3′
Human miR-92a-2* (i.e. hsa-miR-92a-2-5p)
(SEQ ID NO: 15)
5′-GGGUGGGGAUUUGUUGCAUUAC-3′
Mouse mature miR-92 (i.e. mmu-miR-92a-3p)
(SEQ ID NO: 16)
5′-UAUUGCACUUGUCCCGGCCUG-3′
Mouse miR-92a-1* (i.e. mmu-miR-92a-1-5p)
(SEQ ID NO: 17)
5′-AGGUUGGGAUUUGUCGCAAUGCU-3′
Mouse miR-92a-2* (i.e. mmu-miR-92a-2-5p)
(SEQ ID NO: 18)
5′-AGGUGGGGAUUGGUGGCAUUAC-3′
Rat mature miR-92 (i.e. rno-miR-92a-3p)
(SEQ ID NO: 19)
5′-UAUUGCACUUGUCCCGGCCUG-3′
Rat miR-92a-1* (i.e. rno-miR-92a-1-5p)
(SEQ ID NO: 20)
5′-AGGUUGGGAUUUGUCGCAAUGCU-3′
Rat miR-92a-2* (i.e. rno-miR-92a-2-5p)
(SEQ ID NO: 21)
5′-AGGUGGGGAUUAGUGCCAUUAC-3′

In some embodiments, an oligonucleotide inhibitor of miR-92 is an inhibitor of miR-92 (e.g., miR-92a-3p, miR-92a-1-5p, miR-92a-2-5p). In one embodiment, an oligonucleotide inhibitor of miR-92 is an inhibitor of mature miR-92 (e.g., hsa-miR-92a-3p).

The sequence of an oligonucleotide inhibitor of miR-92 according to the invention is sufficiently complementary to a sequence of miR-92 as to hybridize to miR-92 under physiological conditions and inhibit the activity or function of miR-92 in a cell or cells of a subject. For example, in some embodiments, the oligonucleotide inhibitor can consist of, consist essentially of or comprise a sequence that is at least partially complementary to a mature miR-92 sequence, e.g. at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% complementary to a mature sequence of miR-92. In one embodiment, the oligonucleotide inhibitor (also referred to as antisense oligonucleotide) consists of, consists essentially of or comprises a sequence that is 100% complementary to a mature miR-92 sequence. In this context, “consists essentially of” includes the optional addition of nucleotides (e.g., one or two) on either or both of the 5′ and 3′ ends, so long as the additional nucleotide(s) do not substantially affect (as defined by an increase in IC50 of no more than 20%) the oligonucleotide's inhibition of miR-92 activity in a cell in a subject or assay as provided herein. It is understood that the sequence of the oligonucleotide inhibitor is considered to be complementary to miR-92 even if the oligonucleotide inhibitor sequence includes a modified nucleotide instead of a naturally-occurring nucleotide. For example, if a mature sequence of miR-92 comprises a guanosine nucleotide at a specific position, the oligonucleotide inhibitor may comprise a modified cytidine nucleotide, such as a locked cytidine nucleotide or 2′-fluoro-cytidine, at the corresponding position. In certain embodiments, the oligonucleotide inhibitor may be designed to have a sequence containing from 1 to 5 (e.g., 1, 2, 3, or 4) mismatches relative to the fully complementary (mature) miR-92 sequence. In certain embodiments, such antisense sequences may be incorporated into shRNAs or other RNA structures containing stem and loop portions, for example.

In some embodiments, the entire sequence of the oligonucleotide inhibitor of miR-19 is fully complementary to a mature sequence of human miR-92a-3p. In various embodiments, the mature sequence of human miR-92a-3p to which the sequence of the oligonucleotide inhibitor of the present invention is partially, substantially, or fully complementary to includes nucleotides 1-22 or nucleotides 2-17 from the 5′ end of SEQ ID NO: 13. In one embodiment, the mature sequence of human miR-92a-3p to which the sequence of the oligonucleotide inhibitor of the present invention is partially, substantially, or fully complementary to includes nucleotides 2-17 from the 5′ end of SEQ ID NO: 13.

In the context of the present invention, the term “oligonucleotide inhibitor”, “antimiR”, “antagonist”, “antisense oligonucleotide or ASO”, “oligomer”, “anti-microRNA oligonucleotide or AMO”, or “mixmer” is used broadly and encompasses an oligomer comprising ribonucleotides, deoxyribonucleotides, modified ribonucleotides, modified deoxyribonucleotides or a combination thereof, that inhibits the activity or function of the target microRNA (miRNA) by fully or partially hybridizing to the miRNA thereby repressing the function or activity of the target miRNA.

The term “about” as used herein is meant to encompass variations of +/−10% and more preferably +/−5%, as such variations are appropriate for practicing the present invention.

Generally, the length of the oligonucleotide inhibitors of the present invention (e.g., oligonucleotide inhibitors of miR-19 or miR-92) can be such that the oligonucleotide reduces target miRNA (e.g., miR-19 or miR-92) activity or function. The oligonucleotide inhibitors of miR-19 and/or miR-92 as provided herein can be from 8 to 20 nucleotides in length, from 15 to 50 nucleotides in length, from 18 to 50 nucleotides in length, from 10 to 18 nucleotides in length, or from 11 to 16 nucleotides in length. The oligonucleotide inhibitor of miR-19 or miR-92 can, in some embodiments, be about 8, about 9, about 10, about 11, about 12, about 13, about 14, about 15, about 16, about 17, or about 18 nucleotides in length. In one embodiment, the present invention provides an oligonucleotide inhibitor of miR-19 or miR-92 that has a length of 11 to 16 nucleotides. In various embodiments, the oligonucleotide inhibitor targeting miR-19 or miR-92 is 11, 12, 13, 14, 15, or 16 nucleotides in length. In one embodiment, the oligonucleotide inhibitor of miR-19 or miR-92 has a length of 12 nucleotides. In some embodiments, the oligonucleotide inhibitor of miR-19 or miR-92 is at least 16 nucleotides in length.

The oligonucleotide inhibitors of the present invention (e.g., oligonucleotide inhibitors of miR-19 and/or miR-92) can comprise one or more locked nucleic acid (LNAs) residues, or “locked nucleotides.” The oligonucleotide inhibitors of the present invention can contain one or more locked nucleic acid (LNAs) residues, or “locked nucleotides.” LNAs are described, for example, in U.S. Pat. Nos. 6,268,490, 6,316,198, 6,403,566, 6,770,748, 6,998,484, 6,670,461, and 7,034,133, all of which are hereby incorporated by reference in their entireties. LNAs are modified nucleotides or ribonucleotides that contain an extra bridge between the 2′ and 4′ carbons of the ribose sugar moiety resulting in a “locked” conformation, and/or bicyclic structure. In one embodiment, the oligonucleotide comprises or contains one or more LNAs having the structure shown by structure A below. Alternatively or in addition, the oligonucleotide may comprise or contain one or more LNAs having the structure shown by structure B below. Alternatively or in addition, the oligonucleotide can comprise or contain one or more LNAs having the structure shown by structure C below.

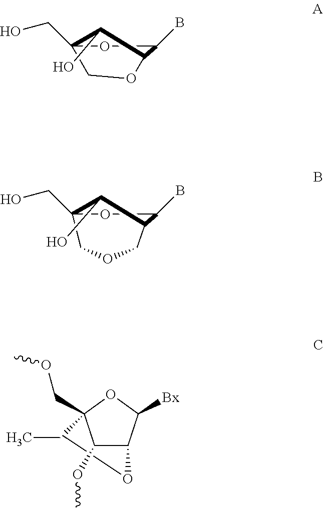

When referring to substituting a DNA or RNA nucleotide by its corresponding locked nucleotide in the context of the present invention, the term “corresponding locked nucleotide” is intended to mean that the DNA/RNA nucleotide has been replaced by a locked nucleotide containing the same naturally-occurring nitrogenous base as the DNA/RNA nucleotide that it has replaced or the same nitrogenous base that is chemically modified. For example, the corresponding locked nucleotide of a DNA nucleotide containing the nitrogenous base C may contain the same nitrogenous base C or the same nitrogenous base C that is chemically modified, such as 5-methylcytosine.

The term “non-locked nucleotide” refers to a nucleotide different from a locked-nucleotide, i.e. the term “non-locked nucleotide” includes a DNA nucleotide, an RNA nucleotide as well as a modified nucleotide where a base and/or sugar is modified except that the modification is not a locked modification.

Other suitable locked nucleotides that can be incorporated in the oligonucleotides of the present invention include those described in U.S. Pat. Nos. 6,403,566 and 6,833,361, both of which are hereby incorporated by reference in their entireties.

In exemplary embodiments, the locked nucleotides have a 2′ to 4′ methylene bridge, as shown in structure A, for example. In other embodiments, the bridge comprises a methylene or ethylene group, which may be substituted, and which may or may not have an ether linkage at the 2′ position.

The oligonucleotide inhibitors of the present invention (e.g., oligonucleotide inhibitors of miR-19 and/or miR-92) as provided herein can generally contain at least about 1, at least about 2, at least about 3, at least about 4, at least about 5, at least about 7, or at least about 9 LNAs. In some embodiments, the oligonucleotide inhibitors of the present invention (e.g., oligonucleotide inhibitors of miR-19 and/or miR-92) comprise a mix of LNA and non-locked nucleotides. For example, the oligonucleotide inhibitors of the present invention (e.g., oligonucleotide inhibitors of miR-19 and/or miR-92) may contain at least five or at least seven or at least nine locked nucleotides, and at least one non-locked nucleotide. Generally, the number and position of LNAs is such that the oligonucleotide inhibitors of the present invention (e.g., oligonucleotide inhibitors of miR-19 and/or miR-92) reduce mRNA or miRNA function or activity. In certain embodiments, the oligonucleotide does not contain a stretch of nucleotides with more than three contiguous LNAs. For example, the oligonucleotide comprises no more than three contiguous LNAs. In these or other embodiments, the oligonucleotide inhibitors of the present invention (e.g., oligonucleotide inhibitors of miR-19 and/or miR-92) can comprise a region or sequence that is substantially or completely complementary to a miRNA seed region (i.e., miR-19 seed region or miR-92 seed region), in which the region or sequence comprises at least two, at least three, at least four, or at least five locked nucleotides. In yet another embodiment, the oligonucleotide inhibitors of the present invention (e.g., oligonucleotide inhibitors of miR-19 and/or miR-92) can comprise a LNA at the 5′ end of the sequence, a LNA at the 3′ end of the sequence, or both a LNA at the 5′ end and 3′ end. For example, the oligonucleotide inhibitors of the present invention (e.g., miR-19 or miR-92) can comprise a sequence of nucleotides in which the sequence comprises at least five LNAs, a LNA at the 5′ end of the sequence, a LNA at the 3′ end of the sequence, or any combination thereof. In one embodiment, the oligonucleotide inhibitor comprises a sequence of nucleotides in which the sequence comprises at least five LNAs, a LNA at the 5′ end of the sequence, a LNA at the 3′ end of the sequence, or any combination thereof, wherein three or fewer of the nucleotides are contiguous LNAs.

In certain embodiments, the oligonucleotide inhibitors of the present invention (e.g., oligonucleotide inhibitors of miR-19 and/or miR-92) contain at least 1, at least 2, at least 3, at least 4, or at least 5 DNA nucleotides. In one embodiment, the oligonucleotide inhibitor comprises at least one LNA, wherein each non-locked nucleotide in the oligonucleotide inhibitor is a DNA nucleotide. In one embodiment, the oligonucleotide inhibitor comprises at least two LNAs, wherein each non-locked nucleotide in the oligonucleotide inhibitor is a DNA nucleotide. In one embodiment, at least the second nucleotide from the 5′ end of the oligonucleotide inhibitor is a DNA nucleotide. In one embodiment, at least 1, at least 2, at least 3, at least 4, or at least 5 DNA nucleotides in an oligonucleotide as provided herein contains a nitrogenous base that is chemically modified. In one embodiment, the second nucleotide from the 5′ end of an oligonucleotide inhibitor as provided herein contains a nitrogenous base that is chemically modified. The chemically modified nitrogenous base can be 5-methylcytosine. In one embodiment, the second nucleotide from the 5′ end is a 5-methylcytosine. In one embodiment, an oligonucleotide inhibitor as provided herein comprises a 5-methylcytosine at each LNA that is a cytosine.

In some embodiments, for non-locked nucleotides in oligonucleotide inhibitors of the present invention, the nucleotide may contain a 2′ modification with respect to a 2′ hydroxyl. For example, the 2′ modification may be 2′ deoxy. Incorporation of 2′-modified nucleotides in antisense oligonucleotides of the present invention may increase resistance of the oligonucleotides to nucleases. Incorporation of 2′-modified nucleotides in antisense oligonucleotides may increase their thermal stability with complementary RNA. Incorporation of 2′-modified nucleotides in antisense oligonucleotides may increase both resistance of the oligonucleotides to nucleases and their thermal stability with complementary RNA. Various modifications at the 2′ positions may be independently selected from those that provide increased nuclease sensitivity, without compromising molecular interactions with the RNA target or cellular machinery. Such modifications may be selected on the basis of their increased potency in vitro, ex vivo or in vivo. Exemplary methods for determining increased potency (e.g., IC50) for miR-19 and/or miR-92 inhibition are described herein, including, but not limited to, the dual luciferase assay and in vivo miRNA expression or target de-repression.

In some embodiments, the 2′ modification may be independently selected from O-alkyl (which may be substituted), halo, and deoxy (H). Substantially all, or all, nucleotide 2′ positions of the non-locked nucleotides may be modified in certain embodiments, e.g., as independently selected from O-alkyl (e.g., O-methyl), halo (e.g., fluoro), deoxy (H), and amino. For example, the 2′ modifications may each be independently selected from O-methyl (OMe) and fluoro (F). In exemplary embodiments, purine nucleotides each have a 2′ OMe and pyrimidine nucleotides each have a 2′-F. In certain embodiments, from one to about five 2′ positions, or from about one to about three 2′ positions are left unmodified (e.g., as 2′ hydroxyls).

2′ modifications in accordance with the invention can also include small hydrocarbon substituents. The hydrocarbon substituents include alkyl, alkenyl, alkynyl, and alkoxyalkyl, where the alkyl (including the alkyl portion of alkoxy), alkenyl and alkynyl may be substituted or unsubstituted. The alkyl, alkenyl, and alkynyl may be C1 to C10 alkyl, alkenyl or alkynyl, such as C1, C2, or C3. The hydrocarbon substituents may include one or two or three non-carbon atoms, which may be independently selected from nitrogen (N), oxygen (O), and/or sulfur (S). The 2′ modifications may further include the alkyl, alkenyl, and alkynyl as O-alkyl, O-alkenyl, and O-alkynyl.

Exemplary 2′ modifications in accordance with the invention can include 2′-O-alkyl (C1-3 alkyl, such as 2′OMe or 2′OEt), 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) substitutions.

In certain embodiments, an oligonucleotide inhibitor provided herein contains at least one 2′-halo modification (e.g., in place of a 2′ hydroxyl), such as 2′-fluoro, 2′-chloro, 2′-bromo, and 2′-iodo. In some embodiments, the 2′ halo modification is fluoro. The oligonucleotide inhibitor may contain from 1 to about 5 2′-halo modifications (e.g., fluoro), or from 1 to about 3 2′-halo modifications (e.g., fluoro). In some embodiments, the oligonucleotide inhibitor contains all 2′-fluoro nucleotides at non-locked positions, or 2′-fluoro on all non-locked pyrimidine nucleotides. In certain embodiments, the 2′-fluoro groups are independently di-, tri-, or un-methylated.

The oligonucleotide inhibitor as provided herein may have one or more 2′-deoxy modifications (e.g., H for 2′ hydroxyl), and in some embodiments, contains from 2 to about 10 2′-deoxy modifications at non-locked positions, or contains 2′ deoxy at all non-locked positions.

In some embodiments, an oligonucleotide inhibitor provided herein contains 2′ positions modified as 2′OMe in non-locked positions. Alternatively, non-locked purine nucleotides can be modified at the 2′ position as 2′OMe, with non-locked pyrimidine nucleotides modified at the 2′ position as 2′-fluoro.

In exemplary embodiments, an oligonucleotide inhibitor provided herein contains 2′ positions modified as 2′OMe in non-locked positions. Alternatively, non-locked purine nucleotides can be modified at the 2′ position as 2′ OMe, with non-locked pyrimidine nucleotides modified at the 2′ position as 2′-fluoro.

In certain embodiments, an oligonucleotide inhibitor provided herein further comprises at least one terminal modification or “cap.” The cap may be a 5′ and/or a 3′-cap structure. The terms “cap” or “end-cap” include chemical modifications at either terminus of the oligonucleotide (with respect to terminal ribonucleotides), and includes modifications at the linkage between the last two nucleotides on the 5′ end and the last two nucleotides on the 3′ end. The cap structure as described herein may increase resistance of the oligonucleotide to exonucleases without compromising molecular interactions with the miRNA target (i.e. miR-19) or cellular machinery. Such modifications may be selected on the basis of their increased potency in vitro or in vivo. The cap can be present at the 5′-terminus (5′-cap) or at the 3′-terminus (3′-cap) or can be present on both ends. In certain embodiments, the 5′- and/or 3′-cap is independently selected from phosphorothioate monophosphate, abasic residue (moiety), phosphorothioate linkage, 4′-thio nucleotide, carbocyclic nucleotide, phosphorodithioate linkage, inverted nucleotide or inverted abasic moiety (2′-3′ or 3′-3′), phosphorodithioate monophosphate, and methylphosphonate moiety. The phosphorothioate or phosphorodithioate linkage(s), when part of a cap structure, are generally positioned between the two terminal nucleotides on the 5′ end and the two terminal nucleotides on the 3′ end.

In certain embodiments, an oligonucleotide inhibitor provided herein has at least one terminal phosphorothioate monophosphate. The phosphorothioate monophosphate may support a higher potency by inhibiting the action of exonucleases. The phosphorothioate monophosphate may be at the 5′ and/or 3′ end of the oligonucleotide. A phosphorothioate monophosphate is defined by the following structures, where B is base, and R is a 2′ modification as described above:

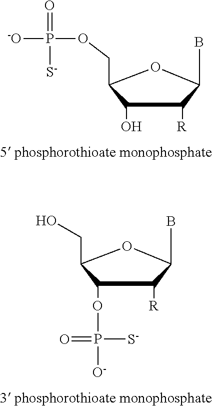

Where the cap structure can support the chemistry of a locked nucleotide, the cap structure may incorporate a LNA as described herein.

Phosphorothioate linkages may be present in some embodiments of oligonucleotide inhibitors provided herein, such as between the last two nucleotides on the 5′ and the 3′ end (e.g., as part of a cap structure), or as alternating with phosphodiester bonds. In these or other embodiments, the oligonucleotide inhibitor may contain at least one terminal abasic residue at either or both the 5′ and 3′ ends. An abasic moiety does not contain a commonly recognized purine or pyrimidine nucleotide base, such as adenosine, guanine, cytosine, uracil or thymine. Thus, such abasic moieties lack a nucleotide base or have other non-nucleotide base chemical groups at the 1′ position. For example, the abasic nucleotide may be a reverse abasic nucleotide, e.g., where a reverse abasic phosphoramidite is coupled via a 5′ amidite (instead of 3′ amidite) resulting in a 5′-5′ phosphate bond. The structure of a reverse abasic nucleoside for the 5′ and the 3′ end of a polynucleotide is shown below.

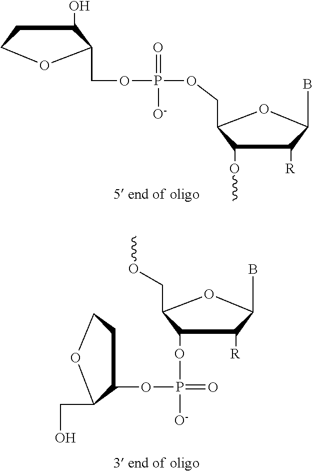

An oligonucleotide inhibitor provided herein may contain one or more phosphorothioate linkages. Phosphorothioate linkages can be used to render oligonucleotides more resistant to nuclease cleavage. For example, the polynucleotide may be partially phosphorothioate-linked, for example, phosphorothioate linkages may alternate with phosphodiester linkages. In certain embodiments, however, the oligonucleotide is fully phosphorothioate-linked. In other embodiments, the oligonucleotide has from one to five or one to three phosphate linkages.

In some embodiments, the nucleotide has one or more carboxamido-modified bases as described in PCT/US11/59588, which is hereby incorporated by reference, including with respect to all exemplary pyrimidine carboxamido modifications disclosed therein with heterocyclic substituents.

The synthesis of oligonucleotides, including modified polynucleotides, by solid phase synthesis is well known and is reviewed in Caruthers et al., Nucleic Acids Symp. Ser. 7:215-23 (1980).

Oligonucleotide inhibitors of the present invention may include modified nucleotides that have a base modification or substitution. The natural or unmodified bases in RNA are the purine bases adenine (A) and guanine (G), and the pyrimidine bases cytosine (C) and uracil (U) (DNA has thymine (T)). Modified bases, also referred to as heterocyclic base moieties, include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (including 5-bromo, 5-trifluoromethyl and other 5-substituted uracils and cytosines), 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. In certain embodiments, oligonucleotide inhibitors targeting miR-19 or miR-92 comprise one or more BSN modifications (i.e., LNAs) in combination with a base modification (e.g. 5-methyl cytidine).

Oligonucleotide inhibitors of the present invention may include nucleotides with modified sugar moieties. Representative modified sugars include carbocyclic or acyclic sugars, sugars having substituent groups at one or more of their 2′, 3′ or 4′ positions and sugars having substituents in place of one or more hydrogen atoms of the sugar. In certain embodiments, the sugar is modified by having a substituent group at the 2′ position. In additional embodiments, the sugar is modified by having a substituent group at the 3′ position. In other embodiments, the sugar is modified by having a substituent group at the 4′ position. It is also contemplated that a sugar may have a modification at more than one of those positions, or that an oligonucleotide inhibitor may have one or more nucleotides with a sugar modification at one position and also one or more nucleotides with a sugar modification at a different position.

Other modifications of oligonucleotide inhibitors to enhance stability and improve efficacy, such as those described in U.S. Pat. No. 6,838,283, which is herein incorporated by reference in its entirety, are known in the art and are suitable for use in the methods of the invention. For instance, to facilitate in vivo delivery and stability, the oligonucleotide inhibitor can be linked to a steroid, such as cholesterol moiety, a vitamin, a fatty acid, a carbohydrate or glycoside, a peptide, or other small molecule ligand at its 3′ end.

In one embodiment, a miR-19 inhibitor of the present invention comprises a sequence selected from Table 1 or a sequence that is at least partially or fully complementary to miR-19 (e.g., miR-19a and/or miR-19b) as provided herein. The miR-19 inhibitor can comprise at least one non-locked nucleotide that is 2′-deoxy, 2′ O-alkyl or 2′ halo modified. In some embodiments, the oligonucleotide comprises at least one LNA that has a 2′ to 4′ methylene bridge. In some embodiments, the oligonucleotide has a 5′ cap structure, 3′ cap structure, or 5′ and 3′ cap structure. In yet other embodiments, the oligonucleotide comprises a pendent lipophilic group. In some embodiments, the miR-19 inhibitor is an oligonucleotide comprising a sequence of 16 nucleotides, wherein the sequence is complementary to miR-19 and comprises no more than three contiguous LNAs, wherein from the 5′ end to the 3′ end, positions 1, 5, 6, 8, 10, 11, 13, 15 and 16 of the sequence are LNAs. In one embodiment, from the 5′ end to the 3′ end, the sequence further comprises a deoxyribonucleic acid (DNA) nucleotide at the second nucleotide position. In yet another embodiment, the oligonucleotide comprises one or more phosphorothioate linkages. In another embodiment, the oligonucleotide is fully phosphorothioate-linked.

In another embodiment, a miR-92 inhibitor of the present invention comprises a sequence selected from Table 2, or a sequence at least partially or fully complementary to miR-92 as provided herein. The miR-92 inhibitor can comprise at least one non-locked nucleotide that is 2′-Deoxy, 2′ O-alkyl or 2′ halo modified. In some embodiments, the miR-92 inhibitor comprises at least one LNA that has a 2′ to 4′ methylene bridge. In some embodiments, the miR-92 inhibitor has a 5′ cap structure, 3′ cap structure, or 5′ and 3′ cap structure. In yet other embodiments, the miR-92 inhibitor comprises a pendent lipophilic group. In some embodiments, the miR-92 inhibitor is an oligonucleotide comprising a sequence of 16 nucleotides, wherein the sequence is complementary to miR-92 and comprises no more than three contiguous LNAs, wherein from the 5′ end to the 3′ end, positions 1, 6, 10, 11, 13 and 16 of the sequence are LNAs. In some embodiments, position 2 from the 5′ end of the oligonucleotide comprising a sequence of 16 nucleotides is a deoxyribonucleic acid (DNA) nucleotide that is 5-methylcytosine. In some embodiments, the miR-92 inhibitor is an oligonucleotide comprising a sequence of 16 nucleotides, wherein the sequence is complementary to miR-92 and comprises no more than three contiguous LNAs, wherein from the 5′ end to the 3′ end, positions 1, 3, 6, 8, 10, 11, 13, 14 and 16 of the sequence are LNAs. In some embodiments, the miR-92 inhibitor is an oligonucleotide comprising a sequence of 16 nucleotides, wherein the sequence is complementary to miR-92 and comprises no more than three contiguous LNAs, wherein from the 5′ end to the 3′ end, positions 1, 5, 6, 8, 10, 11, 13, 15 and 16 of the sequence are LNAs. In some embodiments, the miR-92 inhibitor is an oligonucleotide comprising a sequence of 16 nucleotides, wherein the sequence is complementary to miR-92 and comprises no more than three contiguous LNAs, wherein from the 5′ end to the 3′ end, positions 1, 3, 6, 9, 10, 11, 13, 14 and 16 of the sequence are LNAs. In some embodiments, the oligonucleotide comprises one or more phosphorothioate linkages. In some embodiments, the oligonucleotide is fully phosphorothioate-linked.

As provided herein, an oligonucleotide inhibitor of miR-19 of the present invention can be used alone or in combination with an oligonucleotide inhibitor of miR-92. In one embodiment, the miR-19 inhibitor is selected from Table 1, while the miR-92 inhibitor is selected from Table 2. In Tables 1 and 2, the “+” or “1” indicates the nucleotide is a LNA; “d” indicates the nucleotide is a DNA; “s” indicates a phophorothioate linkage between the two nucelotides; and “mdC” indicates the nucleotide is a 5-methyl cytosine DNA:

TABLE 1
MiR-19 Inhibitors

| SEQ ID | | |
|---|---|---|
| NO. | Alias | Sequence (5′ to 3′) |
| SEQ ID | 19b_LNA_DNA_PS_16 | +TTGC+A+TG+GA+T+TT+ |
| NO: 11 | | GC+A+C |
| | | lTs; dTs; dGs; dCs; lAs; lTs; dGs; |
| | | lGs; dAs; lTs; lTs; dTs; lGs; dCs; |
| | | lAs; lC |
| SEQ ID | 19a_LNA_DNA_PS_16 | +TTGC+A+TA+GA+T+TT+ |
| NO: 12 | | GC+A+C |
| | | lTs; dTs; dGs; dCs; lAs; lTs; dAs; |
| | | lGs; dAs; lTs; lTs; dTs; lGs; dCs; |
| | | lAs; lC |

TABLE 2
MiR-92 Inhibitors

| SEQ | | Sequence (5′ to 3′) |
|---|---|---|
| ID NO. | Alias | (second line of sequence is with linkages notation) |
| SEQ | 92a_LNA_16_PS | +CC+GGG+AC+AA+G+TG+C+AA+T |
| ID NO: | | lCs; dCs; lGs; dGs; dGs; lAs; dCs; lAs; dAs; lGs; lTs; dGs; lCs; lAs; dAs; lT |
| 22 | | |
| SEQ | 92a_LNA_16_1 | +CCGG+G+AC+AA+G+TG+CA+A+T |
| ID NO: | | lCs; dCs; dGs; dGs; lGs; lAs; dCs; lAs; dAs; lGs; lTs; dGs; lCs; dAs; lAs; lT |
| 23 | | |
| SEQ | 92a_LNA_16_4 | +CC+GGG+ACA+A+G+TG+C+AA+T |
| ID | | lCs; dCs; lGs; dGs; dGs; lAs; dCs; dAs; lAs; lGs; lTs; dGs; lCs; lAs; dAs; lT |
| NO: 24 | | |
| SEQ | 92a_Tiny_LNA | lAs; lGs; lTs; lGs; lCs; lAs; lAs; lT; |
| ID NO: | | +A+G+T+G+C+A+A+T |
| 25 | | |
| SEQ | 92a_LNA_16_2 | lCs; dCs; lGs; lGs; dGs; dAs; lCs; lAs; dAs; lGs; dTs; lGs; dCs; lAs; dAs; lT |
| ID NO: | | +CC+G+GGA+C+AA+GT+GC+AA+T |
| 26 | | |
| SEQ | 92a_LNA_16_3 | lCs; dCs; dGs; lGs; lGs; dAs; lCs; dAs; lAs; lGs; dTs; lGs; dCs; lAs; dAs; lT |
| ID NO: | | +CCG+G+GA+CA+A+GT+GC+AA+T |
| 27 | | |
| SEQ | 92a_LNA_16_5 | lCs; dCs; lGs; dGs; lGs; dAs; lCs; dAs; lAs; dGs; lTs; dGs; lCs; dAs; lAs; lT |
| ID NO: | | +CC+GG+GA+CA+AG+TG+CA+A+T |
| 28 | | |
| SEQ | 92a_LNA_16_6 | lCs; lCs; dGs; lGs; dGs; lAs; dCs; lAs; dAs; lGs; dTs; lGs; dCs; lAs; dAs; lT |
| ID NO: | | +C+CG+GG+AC+AA+GT+GC+AA+T |
| 29 | | |
| SEQ | 92a_LNA_16_7 | lCs; dCs; dGs; dGs; lGs; dAs; dCs; lAs; dAs; dGs; lTs; lGs; lCs; lAs; lAs; lT |
| ID NO: | | +CCGG+GAC+AAG+T+G+C+A+A+T |
| 30 | | |
| SEQ | 92a_LNA_16_8 | lCs; dCs; dGs; lGs; dGs; lAs; dCs; lAs; dAs; lGs; lTs; dGs; lCs; lAs; dAs; lT |
| ID NO: | | +CCG+GG+AC+AA+G+TG+C+AA+T |
| 31 | | |
| SEQ | 92a_LNA_16_9 | lCs; dCs; lGs; dGs; lGs; dAs; dCs; lAs; dAs; lGs; dTs; lGs; lCs; lAs; dAs; lT |
| ID NO: | | +CC+GG+GAC+AA+GT+G+C+AA+T |
| 32 | | |
| SEQ | 92a_LNA_16_10 | lCs; dCs; lGs; dGs; dGs; lAs; dCs; lAs; dAs; lGs; lTs; dGs; dCs; lAs; lAs; lT |
| ID NO: | | +CC+GGG+AC+AA+G+TGC+A+A+T |
| 33 | | |
| SEQ | 92a_LNA_16_11 | lCs; lCs; lGs; dGs; dGs; lAs; dCs; lAs; dAs; dGs; lTs; dGs; dCs; lAs; lAs; lT |
| ID NO: | | +C+C+GGG+AC+AAG+TGC+A+A+T |
| 34 | | |
| SEQ | 92a_LNA_16_12 | lCs; lCs; lGs; dGs; lGs; dAs; dCs; lAs; dAs; dGs; lTs; dGs; dCs; lAs; lAs; lT |
| ID NO: | | +C+C+GG+GAC+AAG+TGC+A+A+T |
| 35 | | |
| SEQ | 92a_LNA_16_13 | lCs; lCs; lGs; dGs; dGs; lAs; dCs; lAs; dAs; dGs; lTs; dGs; lCs; lAs; dAs; lT |
| ID NO: | | +C+C+GGG+AC+AAG+TG+C+AA+T |
| 36 | | |
| SEQ | 92a_LNA_16_14 | lCs; dCs; lGs; dGs; lGs; dAs; lCs; dAs; dAs; lGs; lTs; dGs; lCs; dAs; lAs; lT |
| ID NO: | | +CC+GG+GA+CAA+G+TG+CA+A+T |
| 37 | | |
| SEQ | 92a_LNA_16_15 | lCs; lCs; dGs; dGs; lGs; dAs; lCs; dAs; lAs; lGs; dTs; lGs; lCs; dAs; dAs; lT |
| ID NO: | | +C+CGG+GA+CA+A+GT+G+CAA+T |
| 38 | | |
| SEQ | 92a_LNA_16_16 | lCs; dCs; lGs; dGs; dGs; lAs; dCs; lAs; lAs; dGs; lTs; dGs; lCs; lAs; dAs; lT |
| ID NO: | | +CC+GGG+AC+A+AG+TG+C+AA+T |
| 39 | | |
| SEQ | 92a_LNA_16_17 | lCs; dCs; lGs; dGs; dGs; lAs; dCs; lAs; dAs; lGs; dTs; lGs; lCs; lAs; dAs; lT |
| ID NO: | | +CC+GGG+AC+AA+GT+G+C+AA+T |
| 40 | | |
| SEQ | 92a_LNA_16_18 | lCs; dCs; lGs; dGs; lGs; dAs; lCs; dAs; dAs; lGs; lTs; dGs; dCs; lAs; lAs; lT |
| ID NO: | | +CC+GG+GA+CAA+G+TGC+A+A+T |
| 41 | | |
| SEQ | 92a_LNA_16_19 | lCs; dCs; lGs; dGs; dGs; lAs; dCs; lAs; dAs; dGs; lTs; dGs; lCs; lAs; lAs; lT |
| ID NO: | | +CC+GGG+AC+AAG+TG+C+A+A+T |
| 42 | | |
| SEQ | 92a_LNA_16_20 | lCs; dCs; lGs; dGs; lGs; dAs; dCs; lAs; dAs; lGs; lTs; dGs; lCs; lAs; dAs; lT |
| ID NO: | | +CC+GG+GAC+AA+G+TG+C+AA+T |
| 43 | | |
| SEQ | 92a_LNA_16_21 | lCs; lCs; dGs; dGs; lGs; dAs; lCs; lAs; dAs; lGs; dTs; dGs; lCs; dAs; lAs; lT |
| ID NO: | | +C+CGG+GA+C+AA+GTG+CA+A+T |
| 44 | | |
| SEQ | 92a_LNA_16_22 | lCs; dCs; lGs; lGs; dGs; dAs; lCs; dAs; lAs; lGs; dTs; lGs; dCs; lAs; dAs; lT |
| ID NO: | | +CC+G+GGA+CA+A+GT+GC+AA+T |
| 45 | | |
| SEQ | 92a_LNA_16_23 | lCs; dCs; dGs; dGs; lGs; lAs; lCs; dAs; dAs; lGs; lTs; dGs; lCs; dAs; lAs; lT |
| ID NO: | | +CCGG+G+A+CAA+G+TG+CA+A+T |
| 46 | | |
| SEQ | 92a_LNA_16_24 | lCs; dCs; dGs; lGs; lGs; dAs; lCs; dAs; dAs; lGs; dTs; lGs; lCs; lAs; dAs; lT |
| ID NO: | | +CCG+G+GA+CAA+GT+G+C+AA+T |
| 47 | | |
| SEQ | 92a_LNA_16_25 | lCs; dCs; dGs; lGs; lGs; dAs; lCs; lAs; dAs; lGs; dTs; lGs; dCs; lAs; dAs; lT |
| ID NO: | | +CCG+G+GA+C+AA+GT+GC+AA+T |
| 48 | | |
| SEQ | 92a_LNA_15_1 | lCs; dGs; dGs; dGs; lAs; dCs; lAs; dAs; lGs; lTs; dGs; lCs; lAs; dAs; lT |
| ID NO: | | +CGGG+AC+AA+G+TG+C+AA+T |
| 49 | | |
| SEQ | 92a_LNA_15_2 | lCs; dGs; dGs; dGs; lAs; dCs; lAs; dAs; lGs; lTs; dGs; lCs; dAs; lAs; lT |
| ID NO: | | +CGGG+AC+AA+G+TG+CA+A+T |
| 50 | | |
| SEQ | 92a_LNA_15_3 | lCs; dGs; lGs; dGs; dAs; lCs; lAs; dAs; lGs; dTs; lGs; dCs; lAs; dAs; lT |
| ID NO: | | +CG+GGA+C+AA+GT+GC+AA+T |
| 51 | | |
| SEQ | 92a_LNA_15_4 | lCs; dGs; dGs; lGs; dAs; lCs; dAs; lAs; lGs; dTs; lGs; dCs; lAs; dAs; lT |
| ID NO: | | +CGG+GA+CA+A+GT+GC+AA+T |
| 52 | | |
| SEQ | 92a_LNA_15_5 | lCs; dGs; dGs; dGs; lAs; dCs; dAs; lAs; lGs; lTs; dGs; lCs; lAs; dAs; lT |
| ID NO: | | +CGGG+ACA+A+G+TG+C+AA+T |
| 53 | | |
| SEQ | 92a_LNA_15_6 | lCs; lGs; dGs; lGs; dAs; lCs; dAs; lAs; dGs; lTs; dGs; lCs; dAs; dAs; lT |
| ID NO: | | +C+GG+GA+CA+AG+TG+CAA+T |
| 54 | | |
| SEQ | 92a_LNA_15_7 | lCs; dGs; lGs; dGs; lAs; dCs; lAs; dAs; lGs; dTs; lGs; dCs; lAs; dAs; lT |
| ID NO: | | +CG+GG+AC+AA+GT+GC+AA+T |
| 55 | | |
| SEQ | 92a_LNA_15_8 | lCs; dGs; dGs; lGs; dAs; dCs; lAs; dAs; dGs; dTs; lGs; lCs; lAs; lAs; lT |
| ID NO: | | +CGG+GAC+AAGT+G+C+A+A+T |
| 56 | | |
| SEQ | 92a_LNA_15_9 | lCs; lGs; dGs; dGs; lAs; dCs; lAs; dAs; lGs; dTs; dGs; dCs; lAs; lAs; lT |
| ID NO: | | +C+GGG+AC+AA+GTGC+A+A+T |
| 57 | | |
| SEQ | 92a_LNA_15_10 | lCs; lGs; dGs; dGs; lAs; dCs; lAs; dAs; dGs; lTs; dGs; dCs; lAs; lAs; lT |
| ID NO: | | +C+GGG+AC+AAG+TGC+A+A+T |
| 58 | | |
| SEQ | 92a_LNA_15_11 | lCs; lGs; dGs; lGs; dAs; dCs; lAs; dAs; dGs; lTs; dGs; dCs; lAs; lAs; lT |
| ID NO: | | +C+GG+GAC+AAG+TGC+A+A+T |
| 59 | | |
| SEQ | 92a_LNA_15_12 | lCs; lGs; dGs; dGs; lAs; dCs; lAs; dAs; dGs; lTs; dGs; lCs; lAs; dAs; lT |
| ID NO: | | +C+GGG+AC+AAG+TG+C+AA+T |
| 60 | | |
| SEQ | 92a_LNA_15_13 | lCs; lGs; dGs; lGs; dAs; lCs; dAs; dAs; lGs; dTs; dGs; lCs; dAs; lAs; lT |
| ID NO: | | +C+GG+GA+CAA+GTG+CA+A+T |
| 61 | | |
| SEQ | 92a_LNA_15_14 | lCs; dGs; dGs; lGs; dAs; lCs; dAs; lAs; lGs; dTs; lGs; lCs; dAs; dAs; lT |
| ID NO: | | +CGG+GA+CA+A+GT+G+CAA+T |
| 62 | | |
| SEQ | 92a_LNA_15_15 | lCs; lGs; dGs; dGs; lAs; dCs; dAs; lAs; dGs; lTs; dGs; lCs; lAs; dAs; lT |
| ID NO: | | +C+GGG+ACA+AG+TG+C+AA+T |
| 63 | | |
| SEQ | 92a_LNA_15_16 | lCs; lGs; dGs; dGs; lAs; dCs; lAs; dAs; lGs; dTs; lGs; dCs; lAs; dAs; lT |
| ID NO: | | +C+GGG+AC+AA+GT+GC+AA+T |
| 64 | | |
| SEQ | 92a_LNA_15_17 | lCs; lGs; dGs; dGs; dAs; lCs; dAs; dAs; lGs; lTs; dGs; dCs; lAs; lAs; lT |
| ID NO: | | +C+GGGA+CAA+G+TGC+A+A+T |
| 65 | | |
| SEQ | 92a_LNA_15_18 | lCs; lGs; dGs; dGs; lAs; dCs; lAs; dAs; dGs; lTs; dGs; lCs; dAs; lAs; lT |
| ID NO: | | +C+GGG+AC+AAG+TG+CA+A+T |
| 66 | | |
| SEQ | 92a_LNA_15_19 | lCs; lGs; dGs; lGs; dAs; dCs; lAs; dAs; lGs; dTs; dGs; lCs; lAs; dAs; lT |
| ID NO: | | +C+GG+GAC+AA+GTG+C+AA+T |
| 67 | | |
| SEQ | 92a_LNA_15_20 | lCs; dGs; dGs; lGs; dAs; lCs; lAs; dAs; lGs; dTs; dGs; lCs; dAs; lAs; lT |
| ID NO: | | +CGG+GA+C+AA+GTG+CA+A+T |
| 68 | | |
| SEQ | 92a_LNA_15_21 | lCs; dGs; lGs; dGs; dAs; lCs; dAs; lAs; lGs; dTs; lGs; dCs; lAs; dAs; lT |
| ID NO: | | +CG+GGA+CA+A+GT+GC+AA+T |
| 69 | | |
| SEQ | 92a_LNA_15_22 | lCs; dGs; dGs; lGs; lAs; lCs; dAs; dAs; lGs; dTs; dGs; lCs; dAs; lAs; lT |
| ID NO: | | +CGG+G+A+CAA+GTG+CA+A+T |
| 70 | | |
| SEQ | 92a_LNA_15_23 | lCs; dGs; dGs; lGs; dAs; lCs; dAs; dAs; lGs; dTs; lGs; lCs; lAs; dAs; lT |
| ID NO: | | +CGG+GA+CAA+GT+G+C+AA+T |
| 71 | | |
| SEQ | 92a_LNA_15_24 | lCs; dGs; dGs; lGs; dAs; lCs; lAs; dAs; lGs; dTs; lGs; dCs; lAs; dAs; lT |
| ID NO: | | +CGG+GA+C+AA+GT+GC+AA+T |
| 72 | | |
| SEQ | | lCs; dCs; lGs; lGs; lGs; lAs; dCs; lAs; dAs; lGs; dTs; dGs; lCs; dAs; dAs; lT |
| ID NO: | | +CC+G+G+G+AC+AA+GTG+CAA+T |
| 73 | | |
| SEQ | | lCs; dCs; lGs; lGs; dGs; lAs; dCs; lAs; lAs; lGs; dTs; dGs; lCs; dAs; lAs; dT |
| ID NO: | | +CC+G+GG+AC+A+A+GTG+CA+AT |
| 74 | | |
| SEQ | | lCs; dCs; lGs; lGs; lGs; lAs; dCs; lAs; dAs; lGs; dTs; dGs; lCs; dAs; lAs; dT |
| ID NO: | | +CC+G+G+G+AC+AA+GTG+CA+AT |
| 75 | | |
| SEQ | | lCs; dCs; dGs; dGs; lGs; lAs; dCs; dAs; lAs; lGs; lTs; dGs; lCs; dAs; lAs; lT |
| ID NO: | | +CCGG+G+ACA+A+G+TG+CA+A+T |
| 76 | | |
| SEQ | | lCs; dCs; dGs; dGs; lGs; lAs; dCs; lAs; dAs; lGs; lTs; dGs; lCs; lAs; dAs; lT |
| ID NO: | | +CCGG+G+AC+AA+G+TG+C+AA+T |
| 77 | | |
| SEQ | | lCs; mdCs; dGs; dGs; lGs; lAs; dCs; lAs; dAs; lGs; lTs; dGs; lCs; dAs; lAs; |
| ID NO: | | lT |
| 78 | | +CCGG+G+AC+AA+G+TG+CA+A+T |
| SEQ | | lCs; mdCs; lGs; dGs; dGs; lAs; dCs; dAs; lAs; lGs; lTs; dGs; lCs; lAs; dAs; |
| ID NO: | | lT |
| 79 | | +CC+GGG+ACA+A+G+TG+C+AA+T |
| SEQ | | lCs; mdCs; lGs; dGs; dGs; lAs; dCs; lAs; dAs; lGs; lTs; dGs; lCs; lAs; dAs; |
| ID NO: | | lT |
| 80 | | +CC+GGG+AC+AA+G+TG+C+AA+T |
| SEQ | 92a_LNA_14_1 | lGs; dGs; dGs; lAs; dCs; lAs; dAs; lGs; lTs; dGs; lCs; lAs; dAs; lT |
| ID NO: | | +GGG+AC+AA+G+TG+C+AA+T |
| 81 | | |
| SEQ | 92a_LNA_14_2 | lGs; dGs; dGs; lAs; dCs; lAs; dAs; lGs; lTs; dGs; lCs; dAs; lAs; lT |
| ID NO: | | +GGG+AC+AA+G+TG+CA+A+T |
| 82 | | |
| SEQ | 92a_LNA_14_3 | lGs; lGs; dGs; dAs; lCs; lAs; dAs; lGs; dTs; lGs; dCs; lAs; dAs; lT |
| ID NO: | | +G+GGA+C+AA+GT+GC+AA+T |
| 83 | | |
| SEQ | 92a_LNA_14_4 | lGs; dGs; lGs; dAs; lCs; dAs; lAs; lGs; dTs; lGs; dCs; lAs; dAs; lT |
| ID NO: | | +GG+GA+CA+A+GT+GC+AA+T |
| 84 | | |
| SEQ | 92a_LNA_14_5 | lGs; dGs; dGs; lAs; dCs; dAs; lAs; lGs; lTs; dGs; lCs; lAs; dAs; lT |
| ID NO: | | +GGG+ACA+A+G+TG+C+AA+T |
| 85 | | |
| SEQ | 92a_LNA_14_6 | lGs; dGs; lGs; dAs; lCs; dAs; lAs; dGs; lTs; dGs; lCs; dAs; dAs; lT |
| ID NO: | | +GG+GA+CA+AG+TG+CAA+T |
| 86 | | |
| SEQ | 92a_LNA_14_7 | lGs; lGs; dGs; lAs; dCs; lAs; dAs; lGs; dTs; lGs; dCs; lAs; dAs; lT |
| ID NO: | | +G+GG+AC+AA+GT+GC+AA+T |
| 87 | | |
| SEQ | 92a_LNA_14_8 | lGs; dGs; lGs; dAs; dCs; lAs; dAs; dGs; dTs; lGs; lCs; lAs; lAs; lT |
| ID NO: | | +GG+GAC+AAGT+G+C+A+A+T |
| 88 | | |
| SEQ | 92a_LNA_14_9 | lGs; dGs; dGs; lAs; dCs; lAs; dAs; lGs; dTs; dGs; dCs; lAs; lAs; lT |
| ID NO: | | +GGG+AC+AA+GTGC+A+A+T |
| 89 | | |
| SEQ | 92a_LNA_14_10 | lGs; dGs; dGs; lAs; dCs; lAs; dAs; dGs; lTs; dGs; dCs; lAs; lAs; lT |
| ID NO: | | +GGG+AC+AAG+TGC+A+A+T |
| 90 | | |
| SEQ | 92a_LNA_14_11 | lGs; dGs; lGs; dAs; dCs; lAs; dAs; dGs; lTs; dGs; dCs; lAs; lAs; lT |
| ID NO: | | +GG+GAC+AAG+TGC+A+A+T |
| 91 | | |
| SEQ | 92a_LNA_14_12 | lGs; dGs; dGs; lAs; dCs; lAs; dAs; dGs; lTs; dGs; lCs; lAs; dAs; lT |
| ID NO: | | +GGG+AC+AAG+TG+C+AA+T |
| 92 | | |
| SEQ | 92a_LNA_14_13 | lGs; dGs; lGs; dAs; lCs; dAs; dAs; lGs; dTs; dGs; lCs; dAs; lAs; lT |
| ID NO: | | +GG+GA+CAA+GTG+CA+A+T |
| 93 | | |
| SEQ | 92a_LNA_14_14 | lGs; dGs; lGs; dAs; lCs; dAs; lAs; lGs; dTs; lGs; lCs; dAs; dAs; lT |
| ID NO: | | +GG+GA+CA+A+GT+G+CAA+T |
| 94 | | |
| SEQ | 92a_LNA_14_15 | lGs; dGs; dGs; lAs; dCs; dAs; lAs; dGs; lTs; dGs; lCs; lAs; dAs; lT |
| ID NO: | | +GGG+ACA+AG+TG+C+AA+T |
| 95 | | |
| SEQ | 92a_LNA_14_16 | lGs; dGs; dGs; lAs; dCs; lAs; dAs; lGs; dTs; lGs; dCs; lAs; dAs; lT |
| ID NO: | | +GGG+AC+AA+GT+GC+AA+T |
| 96 | | |
| SEQ | 92a_LNA_14_17 | lGs; dGs; dGs; dAs; lCs; dAs; dAs; lGs; lTs; dGs; dCs; lAs; lAs; lT |
| ID NO: | | +GGGA+CAA+G+TGC+A+A+T |
| 97 | | |
| SEQ | 92a_LNA_14_18 | lGs; dGs; dGs; lAs; dCs; lAs; dAs; dGs; lTs; dGs; lCs; dAs; lAs; lT |
| ID NO: | | +GGG+AC+AAG+TG+CA+A+T |
| 98 | | |
| SEQ | 92a_LNA_14_19 | lGs; dGs; lGs; dAs; dCs; lAs; dAs; lGs; dTs; dGs; lCs; lAs; dAs; lT |
| ID NO: | | +GG+GAC+AA+GTG+C+AA+T |
| 99 | | |
| SEQ | 92a_LNA_14_20 | lGs; dGs; lGs; dAs; lCs; lAs; dAs; lGs; dTs; dGs; lCs; dAs; lAs; lT |
| ID NO: | | +GG+GA+C+AA+GTG+CA+A+T |
| 100 | | |
| SEQ | 92a_LNA_14_21 | lGs; lGs; dGs; dAs; lCs; dAs; lAs; lGs; dTs; lGs; dCs; lAs; dAs; lT |
| ID NO: | | +G+GGA+CA+A+GT+GC+AA+T |
| 101 | | |
| SEQ | 92a_LNA_14_22 | lGs; dGs; lGs; lAs; lCs; dAs; dAs; lGs; dTs; dGs; lCs; dAs; lAs; lT |
| ID NO: | | +GG+G+A+CAA+GTG+CA+A+T |
| 102 | | |
| SEQ | 92a_LNA_14_23 | lGs; dGs; lGs; dAs; lCs; dAs; dAs; lGs; dTs; lGs; lCs; lAs; dAs; lT |
| ID NO: | | +GG+GA+CAA+GT+G+C+AA+T |
| 103 | | |
| SEQ | 92a_LNA_14_24 | lGs; dGs; lGs; dAs; lCs; lAs; dAs; lGs; dTs; lGs; dCs; lAs; dAs; lT |
| ID NO: | | +GG+GA+C+AA+GT+GC+AA+T |
| 104 | | |
| SEQ | 92a_LNA_13_1 | lGs; dGs; lAs; dCs; lAs; dAs; lGs; lTs; dGs; lCs; lAs; dAs; lT |
| ID NO: | | +GG+AC+AA+G+TG+C+AA+T |
| 105 | | |
| SEQ | 92a_LNA_13_2 | lGs; dGs; lAs; dCs; lAs; dAs; lGs; lTs; dGs; lCs; dAs; lAs; lT |
| ID NO: | | +GG+AC+AA+G+TG+CA+A+T |
| 106 | | |
| SEQ | 92a_LNA_13_3 | lGs; dGs; dAs; lCs; lAs; dAs; lGs; dTs; lGs; dCs; lAs; dAs; lT |
| ID NO: | | +GGA+C+AA+GT+GC+AA+T |
| 107 | | |
| SEQ | 92a_LNA_13_4 | lGs; lGs; dAs; lCs; dAs; lAs; lGs; dTs; lGs; dCs; lAs; dAs; lT |
| ID NO: | | +G+GA+CA+A+GT+GC+AA+T |
| 108 | | |
| SEQ | 92a_LNA_13_5 | lGs; dGs; lAs; dCs; dAs; lAs; lGs; lTs; dGs; lCs; lAs; dAs; lT |
| ID NO: | | +GG+ACA+A+G+TG+C+AA+T |
| 109 | | |
| SEQ | 92a_LNA_13_6 | lGs; lGs; dAs; lCs; dAs; lAs; dGs; lTs; dGs; lCs; dAs; dAs; lT |
| ID NO: | | +G+GA+CA+AG+TG+CAA+T |
| 110 | | |
| SEQ | 92a_LNA_13_7 | lGs; dGs; lAs; dCs; lAs; dAs; lGs; dTs; lGs; dCs; lAs; dAs; lT |
| ID NO: | | +GG+AC+AA+GT+GC+AA+T |
| 111 | | |
| SEQ | 92a_LNA_13_8 | lGs; lGs; dAs; dCs; lAs; dAs; dGs; dTs; lGs; lCs; lAs; lAs; lT |
| ID NO: | | +G+GAC+AAGT+G+C+A+A+T |
| 112 | | |
| SEQ | 92a_LNA_13_9 | lGs; dGs; lAs; dCs; lAs; dAs; lGs; dTs; dGs; dCs; lAs; lAs; lT |
| ID NO: | | +GG+AC+AA+GTGC+A+A+T |
| 113 | | |
| SEQ | 92a_LNA_13_10 | lGs; dGs; lAs; dCs; lAs; dAs; dGs; lTs; dGs; dCs; lAs; lAs; lT |
| ID NO: | | +GG+AC+AAG+TGC+A+A+T |
| 114 | | |
| SEQ | 92a_LNA_13_11 | lGs; lGs; dAs; dCs; lAs; dAs; dGs; lTs; dGs; dCs; lAs; lAs; lT |
| ID NO: | | +G+GAC+AAG+TGC+A+A+T |
| 115 | | |
| SEQ | 92a_LNA_13_12 | lGs; dGs; lAs; dCs; lAs; dAs; dGs; lTs; dGs; lCs; lAs; dAs; lT |
| ID NO: | | +GG+AC+AAG+TG+C+AA+T |
| 116 | | |
| SEQ | 92a_LNA_13_13 | lGs; lGs; dAs; lCs; dAs; dAs; lGs; dTs; dGs; lCs; dAs; lAs; lT |
| ID NO: | | +G+GA+CAA+GTG+CA+A+T |
| 117 | | |
| SEQ | 92a_LNA_13_14 | lGs; lGs; dAs; lCs; dAs; lAs; lGs; dTs; lGs; lCs; dAs; dAs; lT |
| ID NO: | | +G+GA+CA+A+GT+G+CAA+T |
| 118 | | |
| SEQ | 92a_LNA_13_15 | lGs; dGs; lAs; dCs; dAs; lAs; dGs; lTs; dGs; lCs; lAs; dAs; lT |
| ID NO: | | +GG+ACA+AG+TG+C+AA+T |
| 119 | | |
| SEQ | 92a_LNA_13_16 | lGs; dGs; lAs; dCs; lAs; dAs; lGs; dTs; lGs; dCs; lAs; dAs; lT |
| ID NO: | | +GG+AC+AA+GT+GC+AA+T |
| 120 | | |
| SEQ | 92a_LNA_13_17 | lGs; dGs; dAs; lCs; dAs; dAs; lGs; lTs; dGs; dCs; lAs; lAs; lT |
| ID NO: | | +GGA+CAA+G+TGC+A+A+T |
| 121 | | |
| SEQ | 92a_LNA_13_18 | lGs; dGs; lAs; dCs; lAs; dAs; dGs; lTs; dGs; lCs; dAs; lAs; lT |
| ID NO: | | +GG+AC+AAG+TG+CA+A+T |
| 122 | | |
| SEQ | 92a_LNA_13_19 | lGs; lGs; dAs; dCs; lAs; dAs; lGs; dTs; dGs; lCs; lAs; dAs; lT |
| ID NO: | | +G+GAC+AA+GTG+C+AA+T |
| 123 | | |
| SEQ | 92a_LNA_13_20 | lGs; lGs; dAs; lCs; lAs; dAs; lGs; dTs; dGs; lCs; dAs; lAs; lT |
| ID NO: | | +G+GA+C+AA+GTG+CA+A+T |
| 124 | | |
| SEQ | 92a_LNA_13_21 | lGs; dGs; dAs; lCs; dAs; lAs; lGs; dTs; lGs; dCs; lAs; dAs; lT |
| ID NO: | | +GGA+CA+A+GT+GC+AA+T |
| 125 | | |
| SEQ | 92a_LNA_13_22 | lGs; lGs; lAs; lCs; dAs; dAs; lGs; dTs; dGs; lCs; dAs; lAs; lT |
| ID NO: | | +G+G+A+CAA+GTG+CA+A+T |
| 126 | | |
| SEQ | 92a_LNA_13_23 | lGs; lGs; dAs; lCs; dAs; dAs; lGs; dTs; lGs; lCs; lAs; dAs; lT |
| ID NO: | | +G+GA+CAA+GT+G+C+AA+T |
| 127 | | |
| SEQ | 92a_LNA_13_24 | lGs; lGs; dAs; lCs; lAs; dAs; lGs; dTs; lGs; dCs; lAs; dAs; lT |
| ID NO: | | +G+GA+C+AA+GT+GC+AA+T |
| 128 | | |
| SEQ | 92a_LNA_12_1 | lGs; lAs; dCs; lAs; dAs; lGs; lTs; dGs; lCs; lAs; dAs; lT |
| ID NO: | | +G+AC+AA+G+TG+C+AA+T |
| 129 | | |
| SEQ | 92a_LNA_12_2 | lGs; lAs; dCs; lAs; dAs; lGs; lTs; dGs; lCs; dAs; lAs; lT |
| ID NO: | | +G+AC+AA+G+TG+CA+A+T |
| 130 | | |
| SEQ | 92a_LNA_12_3 | lGs; dAs; lCs; lAs; dAs; lGs; dTs; lGs; dCs; lAs; dAs; lT |
| ID NO: | | +GA+C+AA+GT+GC+AA+T |
| 131 | | |
| SEQ | 92a_LNA_12_4 | lGs; dAs; lCs; dAs; lAs; lGs; dTs; lGs; dCs; lAs; dAs; lT |
| ID NO: | | +GA+CA+A+GT+GC+AA+T |
| 132 | | |
| SEQ | 92a_LNA_12_5 | lGs; lAs; dCs; dAs; lAs; lGs; lTs; dGs; lCs; lAs; dAs; lT |
| ID NO: | | +G+ACA+A+G+TG+C+AA+T |
| 133 | | |
| SEQ | 92a_LNA_12_6 | lGs; dAs; lCs; dAs; lAs; dGs; lTs; dGs; lCs; dAs; dAs; lT |
| ID NO: | | +GA+CA+AG+TG+CAA+T |
| 134 | | |
| SEQ | 92a_LNA_12_7 | lGs; lAs; dCs; lAs; dAs; lGs; dTs; lGs; dCs; lAs; dAs; lT |
| ID NO: | | +G+AC+AA+GT+GC+AA+T |
| 135 | | |
| SEQ | 92a_LNA_12_8 | lGs; dAs; dCs; lAs; dAs; dGs; dTs; lGs; lCs; lAs; lAs; lT |
| ID NO: | | +GAC+AAGT+G+C+A+A+T |
| 136 | | |
| SEQ | 92a_LNA_12_9 | lGs; lAs; dCs; lAs; dAs; lGs; dTs; dGs; dCs; lAs; lAs; lT |
| ID NO: | | +G+AC+AA+GTGC+A+A+T |
| 137 | | |
| SEQ | 92a_LNA_12_10 | lGs; lAs; dCs; lAs; dAs; dGs; lTs; dGs; dCs; lAs; lAs; lT |
| ID NO: | | +G+AC+AAG+TGC+A+A+T |
| 138 | | |
| SEQ | 92a_LNA_12_11 | lGs; dAs; dCs; lAs; dAs; dGs; lTs; dGs; dCs; lAs; lAs; lT |
| ID NO: | | +GAC+AAG+TGC+A+A+T |
| 139 | | |
| SEQ | 92a_LNA_12_12 | lGs; lAs; dCs; lAs; dAs; dGs; lTs; dGs; lCs; lAs; dAs; lT |
| ID NO: | | +G+AC+AAG+TG+C+AA+T |
| 140 | | |
| SEQ | 92a_LNA_12_13 | lGs; dAs; lCs; dAs; dAs; lGs; dTs; dGs; lCs; dAs; lAs; lT |
| ID NO: | | +GA+CAA+GTG+CA+A+T |
| 141 | | |
| SEQ | 92a_LNA_12_14 | lGs; dAs; lCs; dAs; lAs; lGs; dTs; lGs; lCs; dAs; dAs; lT |
| ID NO: | | +GA+CA+A+GT+G+CAA+T |
| 142 | | |
| SEQ | 92a_LNA_12_15 | lGs; lAs; dCs; dAs; lAs; dGs; lTs; dGs; lCs; lAs; dAs; lT |
| ID NO: | | +G+ACA+AG+TG+C+AA+T |
| 143 | | |
| SEQ | 92a_LNA_12_16 | lGs; lAs; dCs; lAs; dAs; lGs; dTs; lGs; dCs; lAs; dAs; lT |
| ID NO: | | +G+AC+AA+GT+GC+AA+T |
| 144 | | |
| SEQ | 92a_LNA_12_17 | lGs; dAs; lCs; dAs; dAs; lGs; lTs; dGs; dCs; lAs; lAs; lT |
| ID NO: | | +GA+CAA+G+TGC+A+A+T |
| 145 | | |
| SEQ | 92a_LNA_12_18 | lGs; lAs; dCs; lAs; dAs; dGs; lTs; dGs; lCs; dAs; lAs; lT |
| ID NO: | | +G+AC+AAG+TG+CA+A+T |
| 146 | | |
| SEQ | 92a_LNA_12_19 | lGs; dAs; dCs; lAs; dAs; lGs; dTs; dGs; lCs; lAs; dAs; lT |
| ID NO: | | +GAC+AA+GTG+C+AA+T |
| 147 | | |
| SEQ | 92a_LNA_12_20 | lGs; dAs; lCs; lAs; dAs; lGs; dTs; dGs; lCs; dAs; lAs; lT |
| ID NO: | | +GA+C+AA+GTG+CA+A+T |
| 148 | | |
| SEQ | 92a_LNA_12_21 | lGs; dAs; lCs; dAs; lAs; lGs; dTs; lGs; dCs; lAs; dAs; lT |
| ID NO: | | +GA+CA+A+GT+GC+AA+T |
| 149 | | |
| SEQ | 92a_LNA_12_22 | lGs; lAs; lCs; dAs; dAs; lGs; dTs; dGs; lCs; dAs; lAs; lT |
| ID NO: | | +G+A+CAA+GTG+CA+A+T |
| 150 | | |
| SEQ | 92a_LNA_12_23 | lGs; dAs; lCs; dAs; dAs; lGs; dTs; lGs; lCs; lAs; dAs; lT |
| ID NO: | | +GA+CAA+GT+G+C+AA+T |
| 151 | | |
| SEQ | 92a_LNA_12_24 | lGs; dAs; lCs; lAs; dAs; lGs; dTs; lGs; dCs; lAs; dAs; lT |
| ID NO: | | +GA+C+AA+GT+GC+AA+T |
| 152 | | |
| SEQ | | lCs; dCs; lGs; dGs; dGs; lAs; dCs; lAs; dAs; lGs; lTs; dGs; lC; lA; dA; lT |
| ID NO: | | +CC+GGG+AC+AA+G+TG+C+AA+T |
| 153 | | |
| SEQ | | lCs; dCs; lGs; dGs; dGs; lAs; dCs; lAs; dAs; lGs; lTs; dG; lC; lA; dA; lT |
| ID NO: | | +CC+GGG+AC+AA+G+TG+C+AA+T |
| 154 | | |
| SEQ | | lCs; dCs; lGs; dGs; dGs; lAs; dCs; lAs; dA; lG; lT; dG; lC; lA; dA; lT |
| ID NO: | | +CC+GGG+AC+AA+G+TG+C+AA+T |
| 155 | | |
| SEQ | | lCs; dC; lGs; dG; dGs; lA; dCs; lA; dAs; lG; lTs; dG; lCs; lA; dAs; lT |
| ID NO: | | +CC+GGG+AC+AA+G+TG+C+AA+T |
| 156 | | |
| SEQ | | lCs; dC; lG; dGs; dG; lA; dCs; lA; dA; lGs; lT; dG; lCs; lA; dA; lT |
| ID NO: | | +CC+GGG+AC+AA+G+TG+C+AA+T |
| 157 | | |
| SEQ | | lC; dC; lG; dG; dG; lA; dC; lA; dA; lG; lT; dG; lC; lA; dA; lT |
| ID NO: | | +CC+GGG+AC+AA+G+TG+C+AA+T |
| 158 | | |
| SEQ | | lCs; mdCs; lGs; dGs; dGs; lAs; dCs; lAs; dAs; lGs; lTs; dGs; lC; lA; dA; lT |
| ID NO: | | +CC+GGG+AC+AA+G+TG+C+AA+T |
| 159 | | |
| SEQ | | lCs; mdCs; lGs; dGs; dGs; lAs; dCs; lAs; dAs; lGs; lTs; dG; lC; lA; dA; lT |
| ID NO: | | +CC+GGG+AC+AA+G+TG+C+AA+T |
| 160 | | |
| SEQ | | lCs; mdCs; lGs; dGs; dGs; lAs; dCs; lAs; dA; lG; lT; dG; lC; lA; dA; lT |
| ID NO: | | +CC+GGG+AC+AA+G+TG+C+AA+T |
| 161 | | |
| SEQ | | lCs; mdC; lGs; dG; dGs; lA; dCs; lA; dAs; lG; lTs; dG; lCs; lA; dAs; lT |
| ID NO: | | +CC+GGG+AC+AA+G+TG+C+AA+T |
| 162 | | |
| SEQ | | lCs; mdC; lG; dGs; dG; lA; dCs; lA; dA; lGs; lT; dG; lCs; lA; dA; lT |
| ID NO: | | +CC+GGG+AC+AA+G+TG+C+AA+T |
| 163 | | |
| SEQ | | lC; mdC; lG; dG; dG; lA; dC; lA; dA; lG; lT; dG; lC; lA; dA; lT |
| ID NO: | | +CC+GGG+AC+AA+G+TG+C+AA+T |
| 164 | | |
| SEQ | | lCs.dmCs.dGs.dGs.lGs.lAs.dCs.lAs.dAs.lGs.lTs.dGs.lC.dA.lA.lT |
| ID NO: | | +CCGG+G+AC+AA+G+TG+CA+A+T |
| 165 | | |
| SEQ | | lCs.dmCs.dGs.dGs.lGs.lAs.dCs.lAs.dAs.lGs.lTs.dG.lC.dA.lA.lT |
| ID NO: | | +CCGG+G+AC+AA+G+TG+CA+A+T |
| 166 | | |
| SEQ | | lCs.dmCs.dGs.dGs.lGs.lAs.dCs.lAs.dA.lG.lT.dG.lC.dA.lA.lT |
| ID NO: | | +CCGG+G+AC+AA+G+TG+CA+A+T |
| 167 | | |
| SEQ | | lCs.dmC.dGs.dG.lGs.lA.dCs.lA.dAs.lG.lTs.dG.lCs.dA.lAs.lT |
| ID NO: | | +CCGG+G+AC+AA+G+TG+CA+A+T |
| 168 | | |
| SEQ | | lCs.dmC.dG.dGs.lG.lA.dCs.lA.dA.lGs.lT.dG.lCs.dA.lA.lT |
| ID NO: | | +CCGG+G+AC+AA+G+TG+CA+A+T |
| 169 | | |
| SEQ | | lC.dmC.dG.dG.lG.lA.dC.lA.dA.lG.lT.dG.lC.dA.lA.lT |
| ID NO: | | +CCGG+G+AC+AA+G+TG+CA+A+T |
| 170 | | |
| SEQ | | lCs.dmCs.lGs.dGs.dGs.lAs.dCs.dAs.lAs.lGs.lTs.dGs.lC.lA.dA.lT |
| ID NO: | | +CC+GGG+ACA+A+G+TG+C+AA+T |
| 171 | | |
| SEQ | | lCs.dmCs.lGs.dGs.dGs.lAs.dCs.dAs.lAs.lGs.lTs.dG.lC.lA.dA.lT |
| ID NO: | | +CC+GGG+ACA+A+G+TG+C+AA+T |
| 172 | | |
| SEQ | | lCs.dmCs.lGs.dGs.dGs.lAs.dCs.dAs.lA.lG.lT.dG.lC.lA.dA.lT |
| ID NO: | | +CC+GGG+ACA+A+G+TG+C+AA+T |
| 173 | | |
| SEQ | | lCs.dmC.lGs.dG.dGs.lA.dCs.dA.lAs.lG.lTs.dG.lCs.lA.dAs.lT |
| ID NO: | | +CC+GGG+ACA+A+G+TG+C+AA+T |
| 174 | | |
| SEQ | | lCs.dmC.lG.dGs.dG.lA.dCs.dA.lA.lGs.lT.dG.lCs.lA.dA.lT |
| ID NO: | | +CC+GGG+ACA+A+G+TG+C+AA+T |
| 175 | | |
| SEQ | | lC.dmC.lG.dG.dG.lA.dC.dA.lA.lG.lT.dG.lC.lA.dA.lT |
| ID NO: | | +CC+GGG+ACA+A+G+TG+C+AA+T |
| 176 | | |
| SEQ | | lCs; dCs; dGs; dGs; lGs; lAs; dCs; dAs; lAs; lGs; lTs; dGs; lCs; dAs; lAs; lT |
| ID NO: | | +CCGG+G+ACA+A+G+TG+CA+A+T |
| 177 | | |
| SEQ | | lCs; dCs; dGs; dGs; lGs; lAs; dCs; lAs; dAs; lGs; lTs; dGs; lCs; lAs; dAs; lT |
| ID NO: | | +CCGG+G+AC+AA+G+TG+C+AA+T |
| 178 | | |
| SEQ | | lCs; dCs; dGs; lGs; lGs; dAs; lCs; dAs; lAs; lGs; dTs; lGs; dCs; lAs; dAs; lT |
| ID NO: | | +CCG+G+GA+CA+A+GT+GC+AA+T |
| 179 | | |

As described herein, administration to a subject of an oligonucleotide inhibitor of a target miRNA (e.g., miR-19 or miR-92) of the present invention reduces or inhibits the activity or function of the target miRNA (e.g., miR-19 or miR-92) in cells of the subject. In some embodiments, the cell is a cardiac or muscle cell. In some embodiments, the cell is a fibrocyte, fibroblast, keratinocyte or endothelial cell. In yet other embodiments, the cell is in vivo or ex vivo. In some embodiments, certain oligonucleotide inhibitors of a target miRNA (e.g., miR-19 or miR-92) of the present invention may show a greater inhibition of the activity or function of the target miRNA (e.g., miR-19 or miR-92) in cells as compared to other miRNA inhibitors of the target miRNA (e.g., miR-19 or miR-92). The term “other miRNA inhibitors” can include nucleic acid inhibitors such as antisense oligonucleotides, antimiRs, antagomiRs, mixmers, gapmers, aptamers, ribozymes, small interfering RNAs, or small hairpin RNAs; antibodies or antigen binding fragments thereof; and/or drugs, which inhibit the function or activity of the target miRNA (e.g., miR-19 or miR-92). It is possible that a particular oligonucleotide inhibitor of a target miRNA of the present invention may show a greater inhibition of the target miRNA (e.g., miR-19 or miR-92) in cells (e.g., muscle cells, cardiac cells, endothelial cells, fibrocytes, fibroblasts, or keratinocytes) compared to other oligonucleotide inhibitors of the target miRNA (e.g., miR-19 or miR-92) of the present invention. The term “greater” as used herein refers to quantitatively more or statistically significantly more. For example, one oligonucleotide inhibitor of miR-19 of the present invention may show higher efficacy as compared to another oligonucleotide inhibitor of miR-19 as measured by the amount of de-repression of a miR-19 target such as frizzled-4 (FZD4) or low-density lipoprotein receptor-related protein 6 (LRP6).

The activity of an oligonucleotide inhibitor of a target miRNA of the present invention in reducing the function or activity of the target miRNA (e.g., miR-19 or miR-92) may be determined in vitro and/or in vivo. For example, when inhibition of miRNA (e.g., miR-19 or miR92) activity is determined in vitro, the activity may be determined using a dual luciferase assay. The dual luciferase assay can be any dual luciferase assay known in the art. The dual luciferase assay can be a commercially available dual luciferase assay. The dual luciferase assay, as exemplified by the commercially available product PsiCHECK™ (Promega), can involve placement of the miR recognition site in the 3′ UTR of a gene for a detectable protein (e.g., renilla luciferase). For example, for assessment of miR-19 inhibitor activity, the construct can be co-expressed with miR-19, such that inhibitor activity can be determined by change in signal. A second gene encoding a detectable protein (e.g., firefly luciferase) can be included on the same plasmid, and the ratio of signals can be determined as an indication of the antimiR (e.g., anti-miR-19) activity of a candidate oligonucleotide. In some embodiments, an oligonucleotide inhibitor of the present invention significantly inhibits such activity, as determined in the dual luciferase activity, at a concentration of about 50 nM or less, or in other embodiments, 40 nM or less, 20 nM or less, or 10 nM or less. For example, for miR-19, the oligonucleotide inhibitor of miR-19 may have an IC50 for inhibition of miR-19 activity of about 50 nM or less, 40 nM or less, 30 nM or less, or 20 nM or less, as determined in the dual luciferase assay.

Alternatively, or in addition, the activity of the oligonucleotide inhibitor of a target miRNA of the present invention in reducing the function or activity of the target miRNA (e.g., miR-19 or miR-92) may be determined in a suitable animal model. Here inhibition (e.g., by at least 50%) of the target miRNA function can be observed at an oligonucleotide inhibitor dose, such as a dose of 50 mg/kg or less, 25 mg/kg or less, 10 mg/kg or less or 5 mg/kg or less. The animal model can be a rodent model (e.g., mouse or rat model). In some embodiments, the activity of the oligonucleotide is determined in an animal model, such as described in WO 2008/016924, which descriptions are hereby incorporated by reference. For example, the oligonucleotide inhibitor may exhibit at least 50% inhibition of the target miRNA, such as a dose of 50 mg/kg or less, 25 mg/kg or less, such as 10 mg/kg or less or 5 mg/kg or less. In such embodiments, the oligonucleotide inhibitor may be dosed, delivered or administered to mice intravenously or subcutaneously or delivered locally such as local injection into muscle, and the oligonucleotide may be formulated in saline. In some embodiments, the oligonucleotide inhibitor(s) may be dosed to mice topically or intradermally (i.e., intradermal injection), such as to a wound (e.g., to the wound margin or wound bed).

In one embodiment, the animal model is a suitable mouse or rat model for diabetes. In one embodiment, the mouse model is a genetically type II diabetic mice such as db/db mice (Jackson Cat #000642 BKS.Cg Dock(Hom) 7m+/+Leprdb/j). In one embodiment, the model uses full thickness cutaneous excisional punch biopsy. In other embodiments, the model utilizes an incision, scald or burn. In such embodiments, the oligonucleotide inhibitor(s) may be dosed to mice intravenously or subcutaneously, or delivered locally such as local injection or topical application to a wound (e.g., the wound margin or wound bed).

In these or other embodiments, the oligonucleotide inhibitors of the present invention can be stable after administration, being detectable in the circulation and/or target organ for at least three weeks, at least four weeks, at least five weeks, or at least six weeks, or more, following administration. Thus, the oligonucleotide inhibitors provided herein (e.g., miR-19 or miR-92) may provide for less frequent administration, lower doses, and/or longer duration of therapeutic effect as compared to other miRNA inhibitors of the target miRNA (e.g., miR-19 or miR-92) as described herein.

The oligonucleotide inhibitors of the present invention may be incorporated within a variety of macromolecular assemblies or compositions alone or in combination. Such complexes for delivery may include a variety of liposomes, nanoparticles, and micelles, formulated for delivery to a patient. The complexes may include one or more fusogenic or lipophilic molecules to initiate cellular membrane penetration. Such molecules are described, for example, in U.S. Pat. No. 7,404,969 and U.S. Pat. No. 7,202,227, which are hereby incorporated by reference in their entireties. Alternatively, the oligonucleotide inhibitors of the present invention may further comprise a pendant lipophilic group to aid cellular delivery, such as those described in WO 2010/129672, which is hereby incorporated by reference.

As previously described herein, compositions of the present invention may employ or comprise a plurality of therapeutic oligonucleotides, including at least one described herein. For example, the composition or formulation may employ or comprise one or all of the miR-19 inhibitors described herein in combination with one or more of the miR-92 inhibitors described herein. In another embodiment of the present invention, a composition of the present invention may comprise a plurality of therapeutic oligonucleotides in combination with one or more other therapeutic modalities. Further to this embodiment, the plurality of therapeutic oligonucleotides can be an oligonucleotide of miR-19 as provided herein in combination with an oligonucleotide inhibitor of miR-92 as provided herein. The other therapeutic modalities can be a pro-angiogenic factor or growth factor. The growth factor can be platelet derived growth factor (PDGF) and/or vascular endothelial growth factor (VEGF). Examples of combination therapies can include any of the foregoing.

Combinations of the oligonucleotide inhibitors provided herein and/or other therapeutic modalities may be achieved with a single composition or pharmacological formulation that includes each agent, or with distinct compositions or formulations each containing at least one agent. The distinct compositions or formulations may be administered simultaneously. Alternatively, the distinct compositions or formulations may be administered sequentially, which can be separated by an interval. For example, a composition using a miR-19 inhibitor may precede or follow administration of the other agent(s) by an interval. The interval can range from seconds, minutes, hours, days, weeks, to months. In some embodiments, a miR-19 inhibitor as provided herein and another agent (e.g., miR-92 inhibitor and/or growth factor such as VEGF or PDGF) are applied separately to the cell in a timeframe or interval configured to permit the other agent (e.g., miR-92 inhibitor and/or growth factor such as VEGF or PDGF) and the miR-19 inhibitor to exert a combined effect on the cell. The combined effect can be advantageous. The combined effect can be advantageous over an effect caused by the other agent (e.g., miR-92 and/or growth factor such as VEGF or PDGF) or the miR-19 inhibitor alone. The miR-19 inhibitor can be an oligonucleotide as provided herein. In such instances, it is contemplated that one would typically contact the cell with the agents within about 12-24 hours of each other, within about 6-12 hours of each other, or with a delay time of only about 12 hours. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In one embodiment, more than one administration of the miR-19 inhibitor or the other agent(s) (e.g., miR-92 inhibitor as provided herein or growth factor such as VEGF or PDGF) can be desired. In this regard, various combinations may be employed. By way of illustration, where the miR-19 inhibitor is “A” and the other agent is “B,” the following permutations based on 3 and 4 total administrations are provided as examples: A/B/A, B/A/B, B/B/A, A/A/B, B/A/A, A/B/B, B/B/B/A, B/B/A/B, A/A/B/B, A/B/A/B, A/B/B/A, B/B/A/A, B/A/B/A, B/A/A/B, B/B/B/A, A/A/A/B, B/A/A/A, A/B/A/A, A/A/B/A, A/B/B/B, B/A/B/B, B/B/A/B. Other combinations are likewise contemplated. Specific examples of other agents and therapies are provided herein. In some embodiments, the other agent is a miR-92 inhibitor as provided herein (e.g., miR-92 inhibitors listed in Table 2).

In one embodiment, a ratio of an amount of a miR-19 inhibitor as provided herein to an amount of another agent (e.g., miR-92 inhibitor as provided herein) in a composition or administered in combination in a method provided herein is from about 99:1, 90:1, 80:1, 70:1, 60:1, 50:1, 40:1, 30:1, 20:1 10:1, 5:1, 3:1, 2:1, 1:1, 1:2, 1:3, 1:5, 1:10, 1:20, 1:30, 1:40, 1:50, 1:60, 1:70, 1:80, 1:90 or 1:99. In one embodiment, a ratio of an amount of a miR-19 inhibitor as provided herein to an amount of another agent (e.g., miR-92 inhibitor as provided herein) in a composition or administered in combination in a method provided herein is 1:1. The ratio can be a mole ratio or molar ratio.

In one embodiment, an amount of a miR-19 inhibitor as provided herein in a composition or administered in a method provided herein is 100-fold, 75-fold, 50-fold, 25-fold, 10-fold, 5-fold, 3-fold, or 2 fold more than or less than an amount of another agent (e.g., miR-92 inhibitor as provided herein) in said composition or administered in combination in said method. In one embodiment, the miR-19 inhibitor as provided herein is administered in an equal amount to the other agent (e.g., miR-92 inhibitor as provided herein).

Also provided herein is an agonist of miR-19 (e.g, miR-19a or miR-19b). In one embodiment, the agonist of miR-19 can be an agent distinct from miR-19 that acts to increase, supplement, or replace the function of miR-19. An agonist of miR-19 can be an oligonucleotide comprising a mature miR-19 sequence. In some embodiments, the oligonucleotide comprises the sequence of the pri-miRNA or pre-miRNA sequence for miR-19. The oligonucleotide comprising the mature miR-19, pre-miR-19, or pri-miR-19 sequence can be single stranded or double stranded. In one embodiment, the miR-19 agonist can be about 15 to about 50 nucleotides in length, about 18 to about 30 nucleotides in length, about 20 to about 25 nucleotides in length, or about 10 to about 14 nucleotides in length. The miR-19 agonist can be at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the mature, pri-miRNA or pre-miRNA sequence of miR-19. The miR-19 agonist that is an oligonucleotide can contain one or more chemical modifications, such as locked nucleic acids, peptide nucleic acids, sugar modifications, such as 2′-O-alkyl (e.g. 2′-O-methyl, 2′-O-methoxyethyl), 2′-fluoro, and 4′ thio modifications, and backbone modifications, such as one or more phosphorothioate, morpholino, or phosphonocarboxylate linkages. In one embodiment, the oligonucleotide that is a miR-19 agonist comprises a miR-19 sequence that is conjugated to cholesterol. The oligonucleotide that is a miR-19 agonist can be a miR-19a, miR-19b or miR-19a/b mimic. In one embodiment, the miR-19 agonist is a miR-19b mimic. In one embodiment, the miR-19b mimic comprises the sequence:

Second/sense/passenger strand
(SEQ ID NO: 180)
5′ mUmCrArGmUmUmUrArGmCmUmUrGrGrAmUmUmUrGrGrAmC
rAChol6-3′
and
First/antisense/guide strand
(SEQ ID NO: 181)
5′ rUrGfUrGfCrArArAfUfCfCrAfUrGfCrArArArAfCfUrG
rAsrUsrU-3′,

in which the abbreviations are defined in Table 3.

TABLE 3
Definitions of Abbreviations

| Nucleotide unit or | | Nucleotide unit or | |
|---|---|---|---|
| modification | Abbreviation | modification | Abbreviation |
| ribo A | rA | ribo A P═S | rAs |
| ribo G | rG | ribo G P═S | rGs |
| ribo C | rC | ribo C P═S | rCs |
| ribo U | rU | ribo U P═S | rUs |
| O-methyl A | mA | O-methyl A P═S | mAs |
| O-methyl G | mG | O-methyl G P═S | mGs |
| O-methyl C | mC | O-methyl C P═S | mCs |
| O-methyl U | mU | O-methyl U P═S | mUs |
| fluoro C | fC | fluoro C P═S | fCs |
| fluoro U | fU | fluoro U P═S | fUs |
| deoxy A | dA | deoxy A P═S | dAs |
| deoxy G | dG | deoxy G P═S | dGs |
| deoxy C | dC | deoxy C P═S | dCs |
| deoxy T | dT | deoxy T P═S | dTs |
| monophosphate | p | | |
| Cholesterol conjugate | Chol6/C6 chol | | |
| with a 6 carbon linker | | | |

A microRNA mimetic or mimic compound according to the invention comprises a first strand and a second strand, wherein the first strand comprises a mature microRNA sequence and the second strand comprises a sequence that is substantially complementary to the first strand and has at least one modified nucleotide. Throughout the disclosure, the term “microRNA mimetic compound” may be used interchangeably with the terms “promiR-19,” “miR-19 agonist,” “miR-19,” “microRNA agonist,” “microRNA mimic,” “miRNA mimic,” or “miR-19 mimic;” the term “first strand” may be used interchangeably with the terms “antisense strand” or “guide strand”; the term “second strand” may be used interchangeably with the term “sense strand” or “passenger strand.” The sequences of the mimics and/or inhibitors can be either ribonucleic acid sequences or deoxyribonucleic acid sequences or a combination of the two (i.e. a nucleic acid comprising both ribonucleotides and deoxyribonucelotides). It is understood that a nucleic acid comprising any one of the sequences described herein will have a thymidine base in place of the uridine base for DNA sequences and a uridine base in place of a thymidine base for RNA sequences.

The present invention further provides pharmaceutical compositions comprising an oligonucleotide or oligonucleotides (e.g., oligonucleotide inhibitors of miR-19 and/or miR-92) disclosed herein. Where clinical applications are contemplated, pharmaceutical compositions can be prepared in a form appropriate for the intended application. Generally, this can entail preparing compositions that are essentially free of pyrogens, as well as other impurities that could be harmful to humans or animals.

In one embodiment, the pharmaceutical composition comprises an effective dose of a miR-19 inhibitor or an effective dose of a miR-19 inhibitor and an effective dose of a miR-92 inhibitor and a pharmaceutically acceptable carrier. The miR-19 inhibitor can be an oligonucleotide that can have a sequence selected from Table 1. The miR-92 inhibitor can be an oligonucleotide that can have a sequence selected from Table 2.

In some embodiments, an “effective dose” is an amount sufficient to effect a beneficial or desired clinical result. An “effective dose” can be an amount sufficient or required to substantially reduce, eliminate or ameliorate a symptom or symptoms of a disease and/or condition. This can be relative to an untreated subject. An “effective dose” can be an amount sufficient or required to slow, stabilize, prevent, or reduce the severity of a pathology in a subject. This can be relative to an untreated subject. An effective dose of an oligonucleotide disclosed herein may be from about 0.001 mg/kg to about 100 mg/kg, about 0.01 mg/kg to about 10 mg/kg, about 0.1 mg/kg to about 10 mg/kg, about 1 mg/kg to about 10 mg/kg, about 2.5 mg/kg to about 50 mg/kg, or about 5 mg/kg to about 25 mg/kg. In some embodiments, an effective dose is an amount of oligonucleotide applied to a wound area. In some embodiments, an effective dose is about 0.01 mg/cm² wound area to about 50 mg/cm² wound area mg/cm² wound area, about 0.02 mg/cm² wound area to about 20 mg/cm² wound area, about 0.1 mg/cm² wound area to about 10 mg/cm² wound area, about 1 mg/cm² wound area to about 10 mg/cm² wound area, about 2.5 mg/cm² wound area to about 50 mg/cm² wound area, or about 5 mg/cm² wound area to about 25 mg/cm² wound area, or about 0.05 to about 25 mg/cm² wound area. The precise determination of what would be considered an effective dose may be based on factors individual to each patient, including their size, age, and nature of the oligonucleotide (e.g. melting temperature, LNA content, etc.). Therefore, dosages can be readily ascertained by those of ordinary skill in the art from this disclosure and the knowledge in the art.

In some embodiments, the methods comprise administering an effective dose of the pharmaceutical composition 1, 2, 3, 4, 5, or 6 times a day. In some embodiments, administration is 1, 2, 3, 4, or 5 times a week. In other embodiments, administration is biweekly or monthly. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

In some embodiments, a composition comprising an oligonucleotide inhibitor provided herein (e.g., miR-19 inhibitor alone or in combination with a miR-92 inhibitor) is suitable for topical application, such as administration at a wound margin or wound bed. In some embodiments, the composition comprises water, saline, PBS or other aqueous solution. In some embodiments, the composition is the form of a lotion, cream, ointment, gel or hydrogel. In some embodiments, the composition suitable for topical application comprises macromolecule complexes, nanocapsules, microspheres, beads, or a lipid-based system (e.g., oil-in-water emulsions, micelles, mixed micelles, and liposomes) as a delivery vehicle. In yet another embodiment, the miR-19 inhibitor (alone or in combination with, for example a miR-92 inhibitor) is in the form of a dry powder or incorporated into a wound dressing.

Colloidal dispersion systems, such as macromolecule complexes, nanocapsules, microspheres, beads, and lipid-based systems including oil-in-water emulsions, micelles, mixed micelles, and liposomes, may be used as delivery vehicles for the oligonucleotide inhibitors of the present invention. Commercially available fat emulsions that are suitable for delivering the nucleic acids of the invention to cardiac and skeletal muscle tissues include Intralipid™ Liposyn™, Liposyn™ II, Liposyn™ III, Nutrilipid, and other similar lipid emulsions. A preferred colloidal system for use as a delivery vehicle in vivo is a liposome (i.e., an artificial membrane vesicle). The preparation and use of such systems is well known in the art. Exemplary formulations are also disclosed in U.S. Pat. Nos. 5,981,505; 6,217,900 6,383,512; 5,783,565; 7,202,227; 6,379,965; 6,127,170; 5,837,533; 6,747,014; and WO03/093449, all of which are hereby incorporated by reference in their entireties.

In certain embodiments, liposomes used for delivery are amphoteric liposomes such SMARTICLES® (Marina Biotech, Inc.) which are described in detail in U.S. Pre-grant Publication No. 20110076322. The surface charge on the SMARTICLES® is fully reversible which make them particularly suitable for the delivery of nucleic acids. SMARTICLES® can be delivered via injection, remain stable, and aggregate free and cross cell membranes to deliver the nucleic acids.

An oligonucleotide provided herein (e.g., oligonucleotide inhibitor of miR-19, miR-19 agonist, or oligonucleotide inhibitor of miR-92) can be expressed in vivo from a vector and/or operably linked to a promoter as known in the art and/or described herein. For example, any of the oligonucleotide inhibitors as provided herein (e.g., miR-19 inhibitor and/or miR-92 inhibitor) can be delivered to the target cell by delivering to the cell an expression vector encoding the oligonucleotide inhibitor as provided herein (e.g., miR-19 inhibitor and/or miR-92 inhibitor). A “vector” is a composition of matter which can be used to deliver a nucleic acid of interest to the interior of a cell. The vector can be any vector known in the art and/or described herein. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, and the like. In one particular embodiment, the viral vector is a lentiviral vector or an adenoviral vector. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In one embodiment, an expression vector for expressing an oligonucleotide inhibitor as provided herein (e.g., miR-19 inhibitor and/or miR-92 inhibitor) comprises a promoter operably linked to a polynucleotide sequence encoding the oligonucleotide inhibitor. The phrase “operably linked” or “under transcriptional control” as used herein means that the promoter is in the correct location and orientation in relation to a polynucleotide to control the initiation of transcription by RNA polymerase and expression of the polynucleotide.

As used herein, a “promoter” refers to a DNA sequence recognized by the synthetic machinery of the cell, or introduced synthetic machinery, required to initiate the specific transcription of a gene. Suitable promoters include, but are not limited to RNA pol I, pol II, pol III, and viral promoters (e.g. human cytomegalovirus (CMV) immediate early gene promoter, the SV40 early promoter, and the Rous sarcoma virus long terminal repeat). In some cases, the promoter may be an inducible promoter. Inducible promoters are known in the art and include, but are not limited to, tetracycline promoter, metallothionein IIA promoter, heat shock promoter, steroid/thyroid hormone/retinoic acid response elements, the adenovirus late promoter, and the inducible mouse mammary tumor virus LTR.

In one embodiment, a single expression vector may encode a miR-19 inhibitor and a miR-92 inhibitor. Here, the miR-19 inhibitor may be driven by a first promoter and the miR-92 inhibitor may driven by a second promoter or the expression vector may comprise a single promoter to control both miRNA inhibitors. In another embodiment, a first expression vector may encode a miR-19 inhibitor, wherein the miR-19 inhibitor is operably linked to a first promoter and a second expression vector may encode a miR-92 inhibitor, wherein the miR-92 inhibitor is operably linked to a second promoter. In any of the above embodiments, a promoter may be an inducible promoter as provided herein. Other combinations of inducible and constitutive promoters for controlling the expression of the miR-19 and miR-92 inhibitors are also contemplated. For instance, a miR-19 inhibitor may be expressed from a vector using a constitutive promoter, while a miR-92 inhibitor may be expressed from a vector using an inducible promoter.

In another embodiment of the invention, a single nucleic acid molecule may be used to inhibit both miR-19 and miR-92 simultaneously. For instance, a single nucleic acid may contain a sequence that is substantially, partially or fully complementary to a mature miR-19 (e.g., miR-19a or miR-19b) sequence (e.g. SEQ ID NO: 3) and a sequence that is substantially, partially or fully complementary to a mature miR-92 sequence (e.g. SEQ ID NO: 13). The single nucleic acid molecule may further comprise a linker between the miR-19 (e.g., miR-19a or miR-19b) and miR-92 targeting sequences. For instance, the single nucleic acid molecule may contain a linker comprising about 1 to about 200 nucleotides, more preferably about 5 to about 100 nucleotides, most preferably about 10 to about 50 nucleotides between the miR-19 (e.g., miR-19a or miR-19b) and miR-92 targeting sequences. In some embodiments, the linker between the miR-19 and miR-92 sequences may be a cleavable linker. The cleavable linker may be a cleavable linker as disclosed in WO2013040429, the contents of which are herein incorporated by reference in their entirety.

In this embodiment, the cleavable linker is a nuclease-cleavable oligonucleotide linker. In some embodiments, the nuclease-cleavable linker contains one or more phosphodiester bonds in the oligonucleotide backbone. For example, the linker may contain a single phosphodiester bridge or 2, 3, 4, 5, 6, 7 or more phosphodiester linkages, for example as a string of 1-10 deoxynucleotides, e.g., dT, or ribonucleotides, e.g., rU, in the case of RNA linkers. In the case of dT or other DNA nucleotides dN in the linker, in certain embodiments the cleavable linker contains one or more phosphodiester linkages. In other embodiments, in the case of rU or other RNA nucleotides rN, the cleavable linker may consist of phosphorothioate linkages only. In contrast to phosphorothioate-linked deoxynucleotides, which are only cleaved slowly by nucleases (thus termed “noncleavable”), phosphorothioate-linked rU undergoes relatively rapid cleavage by ribonucleases and therefore is considered cleavable herein. It is also possible to combine dN and rN into the linker region, which are connected by phosphodiester or phosphorothioate linkages. In other embodiments, the linker can also contain chemically modified nucleotides, which are still cleavable by nucleases, such as, e.g., 2′-O-modified analogs. In particular, 2′-O-methyl or 2′-fluoro nucleotides can be combined with each other or with dN or rN nucleotides. Generally, in the case of nucleotide linkers, the linker is a part of the multimer that is usually not complementary to a target, although it could be. This is because the linker is generally cleaved prior to targeting oligonucleotides action on the target, and therefore, the linker identity with respect to a target is inconsequential. Accordingly, in some embodiments, a linker is an (oligo)nucleotide linker that is not complementary to any of the targets against which the targeting oligonucleotides (e.g., miR-19 and miR-92 targeting sequences) are designed. The cleavable linker can be designed so as to undergo a chemical or enzymatic cleavage reaction. Chemical reactions involve, for example, cleavage in acidic environment (e.g., endosomes), reductive cleavage (e.g., cytosolic cleavage) or oxidative cleavage (e.g., in liver microsomes). The cleavage reaction can also be initiated by a rearrangement reaction. Enzymatic reactions can include reactions mediated by nucleases, peptidases, proteases, phosphatases, oxidases, reductases, etc. For example, a linker can be pH-sensitive, cathepsin-sensitive, or predominantly cleaved in endosomes and/or cytosol. In some embodiments, the cleavable linker is organ- or tissue-specific, for example, liver-specific, kidney-specific, intestine-specific, etc.

Methods of delivering expression constructs and nucleic acids to cells are known in the art and can include, for example, calcium phosphate co-precipitation, electroporation, microinjection, DEAE-dextran, lipofection, transfection employing polyamine transfection reagents, cell sonication, gene bombardment using high velocity microprojectiles, and receptor-mediated transfection.

One will generally desire to employ appropriate salts and buffers to render delivery vehicles stable and allow for uptake by target cells. Pharmaceutical compositions of the present invention can comprise an effective amount of the delivery vehicle comprising the inhibitor polynucleotides (e.g. liposomes or other complexes or expression vectors) dissolved or dispersed in a pharmaceutically acceptable carrier or aqueous medium. The phrases “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the oligonucleotides of the compositions.

The compositions comprising active compounds of the present invention may include classic pharmaceutical preparations known in the art. Administration of these compositions according to the present invention may be via any common route so long as the target tissue is available via that route. This includes oral, nasal, or buccal. Alternatively, administration may be topical or be by intradermal, subcutaneous, intramuscular, intraperitoneal, intraarterial, or intravenous injection. In some embodiments, the pharmaceutical composition is directly injected into lung or cardiac tissue. In another embodiment, compositions comprising oligonucleotide inhibitors as described herein (e.g., oligonucleotide inhibitors of miR-19 and/or miR-92) may be formulated in the form suitable for a topical application such as a cream, ointment, paste, lotion, or gel. In some embodiments, the pharmaceutical composition is directly injected into the wound area. In some embodiments, the pharmaceutical composition is topically applied to the wound area.

Pharmaceutical compositions comprising oligonucleotide inhibitors as described herein may also be administered by catheter systems or systems that isolate coronary/pulmonary circulation for delivering therapeutic agents to the heart and lungs. Various catheter systems for delivering therapeutic agents to the heart and coronary vasculature are known in the art. Some non-limiting examples of catheter-based delivery methods or coronary isolation methods suitable for use in the present invention are disclosed in U.S. Pat. No. 6,416,510; U.S. Pat. No. 6,716,196; U.S. Pat. No. 6,953,466, WO 2005/082440, WO 2006/089340, U.S. Patent Publication No. 2007/0203445, U.S. Patent Publication No. 2006/0148742, and U.S. Patent Publication No. 2007/0060907, which are all herein incorporated by reference in their entireties. Such compositions can be administered as pharmaceutically acceptable compositions as described herein.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use, catheter delivery, or inhalational delivery can include, for example, sterile aqueous solutions or dispersions and sterile powders for the extemporaneous preparation of sterile injectable solutions or dispersions (e.g. aerosols, nebulizer solutions). Generally, these preparations can be sterile and fluid to the extent that easy injectability or aerosolization/nebulization exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, a composition comprising a miR-19 inhibitor or a miR-19 inhibitor and a miR-92 inhibitor is suitable for topical application, such as administration at a wound margin or wound bed. In some embodiments, the composition comprises water, saline, PBS or other aqueous solution. In some embodiments, the miR-19 inhibitor or the miR-19 inhibitor and the miR-92 inhibitor is in a lotion, cream, ointment, gel or hydrogel. In some embodiments, the composition suitable for topical application comprises macromolecule complexes, nanocapsules, microspheres, beads, or a lipid-based system (e.g., oil-in-water emulsions, micelles, mixed micelles, and liposomes) as a delivery vehicle. In yet another embodiment, the miR-19 inhibitor or the miR-19 inhibitor and the miR-92 inhibitor is in the form of a dry powder or incorporated into a wound dressing.

Sterile injectable solutions may be prepared by incorporating the active compounds in an appropriate amount into a solvent along with any other ingredients (for example as enumerated above) as desired, followed by filtered sterilization. The appropriate amount can be an amount above a desired amount in the final preparation in order to account for loss or degradation of the active compound during preparation. The desired amount can be a dose as provided herein. The dose can be an effective dose or a fraction thereof. Generally, dispersions can be prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof. In some embodiments, sterile powders can be administered directly to the subject (i.e. without reconstitution in a diluent), for example, through an insufflator or inhalation device.

In some embodiments, administration of a miR-19 inhibitor alone or in combination with a miR-92 inhibitor is by subcutaneous or intradermal injection, such as to a wound (e.g., a chronic wound, diabetic foot ulcer, venous stasis leg ulcer or pressure sore). Administration may be at the site of a wound, such as to the wound margin or wound bed.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids), or from organic acids (e.g., acetic, oxalic, tartaric, mandelic, and the like). Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like).

Upon formulation, solutions can be preferably administered in a manner compatible with the dosage formulation and in such amount as is therapeutically effective. The formulations may easily be administered in a variety of dosage forms such as injectable solutions, drug release capsules, unit dose inhalers, and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous, intraarterial, and intraperitoneal administration. Preferably, sterile aqueous media can be employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration can, in any event, determine the appropriate dose for the individual subject. Moreover, for human administration, preparations should meet sterility, pyrogenicity, general safety and purity standards as required by the FDA Office of Biologics standards.

Also provided herein is a method for treating, ameliorating, or preventing the progression of a condition in a subject comprising administering a pharmaceutical composition comprising an inhibitor or a combination of inhibitors as disclosed herein. The method generally comprises administering the inhibitor or composition comprising the same to a subject. The term “subject” or “patient” refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In other embodiments, the subject is a human. The subject may have a condition associated with, mediated by, or resulting from, expression of miR-19 (e.g., miR-19a and/or miR-19b), or miR-19 (e.g., miR-19a and/or miR-19b) and miR-92.

In one embodiment, a method of promoting angiogenesis in a subject comprises administering to the subject a miR-19 inhibitor alone or in combination with a miR-92 inhibitor. In one embodiment, the miR-19 inhibitor is an oligonucleotide, such as is selected from Table 1. In one embodiment, the miR-92 inhibitor is an oligonucleotide, such as is selected from Table 2. In some embodiments, the subject suffers from ischemia, myocardial infarction, chronic ischemic heart disease, peripheral or coronary artery occlusion, ischemic infarction, stroke, atherosclerosis, acute coronary syndrome, coronary artery disease, carotid artery disease, diabetes, chronic wound(s), or peripheral artery disease.

In another embodiment, a method of treating or preventing ischemia, myocardial infarction, chronic ischemic heart disease, peripheral or coronary artery occlusion, ischemic infarction, stroke, atherosclerosis, acute coronary syndrome, coronary artery disease, carotid artery disease, or peripheral artery disease in a subject comprises administering to the subject a miR-19 inhibitor alone or in combination with a miR-92 inhibitor. In one embodiment, the miR-19 inhibitor is an oligonucleotide, such as is selected from Table 1. In one embodiment, the miR-92 inhibitor is an oligonucleotide, such as is selected from Table 2.

In one embodiment of the present invention, the method of promoting angiogenesis in a subject in need thereof comprises administering to the subject a miR-19 inhibitor, such as a miR-19 inhibitor as described herein, and another agent that promotes angiogenesis. In one embodiment of the present invention, a method of treating or preventing peripheral artery disease in a subject in need thereof comprises administering to the subject a miR-19 inhibitor, such as a miR-19 inhibitor as described herein. In some embodiments, the method further comprises administering another agent with the miR-19 inhibitor. The other agent may be an inhibitor of miR-92 (e.g., an miR-92 inhibitor listed in Table 2). In some embodiments, the other agent may promote angiogenesis or be an agent used for treating atherosclerosis or peripheral artery disease. The other agent may be a phophodiesterase type 3 inhibitor (such as cilostazol), a statin, an antiplatelet, L-carnitine, propionyl-L-carnitine, pentoxifylline, or naftidrofuryl. The method of treating or preventing peripheral artery disease in a subject in need thereof may also comprise administering antimiR-19 to the subject, in which the subject is also receiving, or will be receiving gene therapy (e.g., with a proangiogenic factor, such as VEGF, FGF, HIF-1α, HGF, or Del-1), cell therapy, and/or antiplatelet therapy. In some embodiments, the method comprises administering a miR-19 inhibitor and an antimicrobial to the subject.

In one embodiment, a method of promoting wound healing in a subject in need thereof comprises administering to the subject a miR-19 inhibitor, such as an antimiR-19 as described herein (e.g., miR-19 inhibitors listed in Table 1). In one embodiment, the subject has diabetes. In some embodiments, the subject has a chronic wound, diabetic foot ulcer, venous stasis leg ulcer or pressure sore. In some embodiments, healing of a chronic wound, diabetic foot ulcer, venous stasis leg ulcer or pressure sore is promoted by administration of a miR-19 inhibitor. In another embodiment, the subject has peripheral vascular disease (e.g., peripheral artery disease). In some embodiments, the method further comprises administering another agent with an antimiR-19. The other agent may be an agent used for treating peripheral vascular disease (e.g., peripheral artery disease), such as described above. In some embodiments, the other agent promotes wound healing or is used to treat diabetes. The other agent may be a pro-angiogenic factor. In some embodiments, the other agent is a growth factor, such as VEGF or PDGF. In some embodiments, the other agent promotes VEGF expression or activity or PDGF expression or activity. In some embodiments, the other agent is an allogeneic skin substitute or biologic dressing, (e.g., Dermagraft® or Apligraf®, available from Organogenesis, Canton, Mass.) or a platelet derived growth factor (PDGF) gel, such as becaplermin (Buchberger et al. Experimental and Clinical Endocrinology and Diabetes 119:472-479 (2011)). In some embodiments, the other agent is a debridement agent or antimicrobial agent. In some embodiments, the other agent comprises an inhibitor of a miRNA located in the miR-17-92 cluster. In one embodiment, the other agent is an inhibitor of miR-92 (e.g., miR-92 inhibitors listed in Table 2).

In one embodiment, administration of a miR-19 inhibitor provides at least about 5%, 10%, 20%, 30%, 40%, 50%, 60%, 70%, 80%, or 90% improvement in wound re-epithelialization or wound closure as compared to a wound not administered the miR-19 inhibitor or any treatment. In some embodiments, administration of a miR-19 inhibitor provides at least about 5%, 10%, 20%, 30%, 40%, 50%, 60%, 70%, 80%, or 90% more granulation tissue formation or neovascularization as compared to a wound not administered the miR-19 inhibitor or any treatment. In one embodiment, administration of a miR-19 inhibitor in combination with a miR-92 inhibitor provides at least about 5%, 10%, 20%, 30%, 40%, 50%, 60%, 70%, 80%, or 90% improvement in wound re-epithelialization or wound closure as compared to a wound not administered the miR-19 inhibitor in combination with the miR-92 inhibitor or any treatment. In some embodiments, administration of a miR-19 inhibitor in combination with a miR-92 inhibitor provides at least about 5%, 10%, 20%, 30%, 40%, 50%, 60%, 70%, 80%, or 90% more granulation tissue formation or neovascularization as compared to a wound not administered the miR-19 inhibitor in combination with the miR-92 inhibitor or any treatment.

In one embodiment, administration of a miR-19 inhibitor provides at least about 5%, 10%, 20%, 30%, 40%, 50%, 60%, 70%, 80%, or 90% improvement in wound re-epithelialization or wound closure as compared to a wound administered an agent known in the art for treating wounds. In some embodiments, administration of a miR-19 inhibitor provides at least about 5%, 10%, 20%, 30%, 40%, 50%, 60%, 70%, 80%, or 90% more granulation tissue formation or neovascularization as compared to a wound administered an agent known in the art for treating wounds. In one embodiment, administration of a miR-19 inhibitor in combination with a miR-92 inhibitor provides at least about 5%, 10%, 20%, 30%, 40%, 50%, 60%, 70%, 80%, or 90% improvement in wound re-epithelialization or wound closure as compared to a wound administered an agent known in the art for treating wounds. In some embodiments, administration of a miR-19 inhibitor in combination with a miR-92 inhibitor provides at least about 5%, 10%, 20%, 30%, 40%, 50%, 60%, 70%, 80%, or 90% more granulation tissue formation or neovascularization as compared to a wound administered an agent known in the art for treating wounds. The agent can be a growth factor such as for example platelet derived growth factor (PDGF) and/or vascular endothelial growth factor (VEGF).

In one embodiment, administration of a miR-19 inhibitor provided herein in combination with a miR-92 inhibitor provided herein provides at least about 5%, 10%, 20%, 30%, 40%, 50%, 60%, 70%, 80%, or 90% improvement in wound re-epithelialization or wound closure as compared to a wound administered either inhibitor alone. In some embodiments, administration of a miR-19 inhibitor in combination with a miR-92 inhibitor provides at least about 5%, 10%, 20%, 30%, 40%, 50%, 60%, 70%, 80%, or 90% more granulation tissue formation or neovascularization as compared to a wound administered either inhibitor alone.

The present invention is also based, in part, on the discovery of genes significantly regulated by miR-19. Accordingly, another aspect of the present invention is a method for evaluating or monitoring the efficacy of a therapeutic for modulating angiogenesis or wound healing in a subject receiving the therapeutic comprising: obtaining a sample from the subject; measuring the expression of one or more genes that are targets of miR-19 (e.g, miR-19a or miR-19b) in the sample; and comparing the expression of the one or more genes that are targets of miR-19 (e.g, miR-19a or miR-19b) to a pre-determined reference level or level of the one or more genes that are targets of miR-19 (e.g, miR-19a or miR-19b) in a control sample, wherein the comparison is indicative of the efficacy of the therapeutic. The one or more genes that are targets of miR-19 (e.g, miR-19a or miR-19b) can comprise a predicted miR-19 binding site. In some embodiments, the one or more genes that are targets of miR-19 (e.g, miR-19a or miR-19b) is FZD4 or LRP6. In some embodiments, the therapeutic modulates miR-19 function and/or activity. The therapeutic can be a miR-19 antagonist, such as a miR-19 inhibitor selected from Table 1. In other embodiments, the therapeutic is a miR-19 agonist, such as a miR-19 mimic. In some embodiments, the therapeutic further modulates the function and/or activity of another miRNA located in the miR-17-2 cluster. In some embodiments, the therapeutic further modulates the function and/or activity of miR-92. In this embodiment, the therapeutic can further comprise a miR-92 antagonist, such as a miR-92 inhibitor selected from Table 2. In some embodiments, the subject suffers from ischemia, myocardial infarction, chronic ischemic heart disease, peripheral or coronary artery occlusion, ischemic infarction, stroke, atherosclerosis, acute coronary syndrome, coronary artery disease, carotid artery disease, or peripheral vascular disease (e.g., peripheral artery disease) and the therapeutic is a miR-19 antagonist as provided herein used alone or in combination with another agent (e.g., a miR-92 antagonist as provided herein). In some embodiments, the subject suffers from diabetes, a chronic wound, diabetic foot ulcer, venous stasis leg ulcer or pressure sore and the therapeutic is a miR-19 antagonist as provided herein used alone or in combination with another agent (e.g., a miR-92 antagonist as provided herein). In embodiments utilizing a miR-92 antagonist, the method can further comprise measuring the expression of one or more targets of miR-92 and comparing the expression or activity of the one or more genes that are targets of miR-92 to a pre-determined reference level or level of the one or more genes that are targets of miR-92 in a control sample, wherein the comparison is indicative of the efficacy of the therapeutic(s).The one or more targets of miR-92 can be one or more of the targets disclosed in US20160208258, the contents of which are hereby incorporated by reference in their entirety.

In some embodiments, the method of evaluating or monitoring the efficacy of a therapeutic for modulating angiogenesis or wound healing in a subject receiving the therapeutic further comprises performing another diagnostic, assay or test evaluating angiogenesis in a subject. In some embodiments, the additional diagnostic assay or test for evaluating or monitoring the efficacy of a therapeutic for modulating angiogenesis is a walk time test, an ankle-bronchial index (ABI), arteriography or angiography on the subject, or a SPECT analysis.

Another aspect of the present invention is a method for selecting a subject for treatment with a therapeutic that modulates miR-19 function and/or activity comprising: obtaining a sample from the subject; measuring the expression of one or more genes that are targets of miR-19 (e.g, miR-19a or miR-19b) in the sample; and comparing the expression or activity of the one or more genes that are targets of miR-19 (e.g, miR-19a or miR-19b) to a pre-determined reference level or level of the one or more genes that are targets of miR-19 (e.g, miR-19a or miR-19b) in a control sample, wherein the comparison is indicative of whether the subject should be selected for treatment with the therapeutic. The one or more genes that are targets of miR-19 (e.g, miR-19a or miR-19b) can comprise a predicted miR-19 binding site. In some embodiments, the one or more genes that are targets of miR-19 (e.g, miR-19a or miR-19b) is FZD4 or LRP6. The therapeutic can be a miR-19 antagonist, such as a miR-19 inhibitor selected from Table 1. In other embodiments, the therapeutic is a miR-19 agonist, such as a miR-19 mimic. In some embodiments, the therapeutic further modulates the function and/or activity of another miRNA located in the miR-17-2 cluster. In some embodiments, the therapeutic further modulates the function and/or activity of miR-92. In this embodiment, the therapeutic can further comprise a miR-92 antagonist, such as a miR-92 inhibitor selected from Table 2. In some embodiments, the subject suffers from ischemia, myocardial infarction, chronic ischemic heart disease, peripheral or coronary artery occlusion, ischemic infarction, stroke, atherosclerosis, acute coronary syndrome, coronary artery disease, carotid artery disease, or peripheral vascular disease (e.g., peripheral artery disease) and the therapeutic is a miR-19 antagonist as provided herein used alone or in combination with another agent (e.g., a miR-92 antagonist as provided herein). In some embodiments, the subject suffers from diabetes, a chronic wound, diabetic foot ulcer, venous stasis leg ulcer or pressure sore and the therapeutic is a miR-19 antagonist as provided herein used alone or in combination with another agent (e.g., a miR-92 antagonist as provided herein). In embodiments utilizing a miR-92 antagonist, the method can further comprise measuring the expression of one or more targets of miR-92 and comparing the expression or activity of the one or more genes that are targets of miR-92 to a pre-determined reference level or level of the one or more genes that are targets of miR-92 in a control sample, wherein the comparison is indicative of whether the subject should be selected for treatment with the therapeutic(s).The one or more targets of miR-92 can be one or more of the targets disclosed in US20160208258, the contents of which are hereby incorporated by reference in their entirety.

In some embodiments, the method for selecting a subject for treatment with a therapeutic that modulates miR-19 function and/or activity comprises obtaining a sample from a subject treated with the therapeutic. In some embodiments, the subject is not treated with the therapeutic and the sample is treated with the therapeutic. In some embodiments, the subject is treated with the therapeutic and the sample is treated with the therapeutic. In some embodiments, the method further comprises performing another diagnostic, assay or test evaluating angiogenesis or wound healing in a subject. In some embodiments, the additional diagnostic assay or test for evaluating angiogenesis is a walk time test, an ankle-bronchial index (ABI), arteriography or angiography on the subject, or a SPECT analysis.

The walk test can be a non-invasive treadmill test to measure the change in maximum or pain-free walk time in response to therapy. The ankle-bronchial index (ABI) can be a pressure measurement taken at the arm and the ankle, such as measured by ultrasound. The index can then be expressed as a ratio of the blood pressure at the ankle compared to the pressure at the arm. The arteriography can be a contrast dye method to measure blood flow through arteries or veins. The SPECT (Single Photon Emission Computed Tomography) analysis can be performed with a 3-D imaging system using radiation to measure blood flow through capillaries.

Also provided herein is a method for evaluating an agent's ability to promote angiogenesis or wound healing comprising: contacting a cell with the agent; measuring the expression or activity of one or more genes that are targets of miR-19 (e.g, miR-19a or miR-19b) in the cell contacted with the agent; and comparing the expression or activity of the one or more genes to a pre-determined reference level or level of the one or more genes in a control sample, wherein the comparison is indicative of the agent's ability to promote angiogenesis. In some embodiments, the method further comprises determining miR-19 function and/or activity in the cell contacted with the agent. In some embodiments, the cell is a mammalian cell. In some embodiments, the cell is a cardiac or muscle cell. In some embodiments, the cell is involved in wound healing. In some embodiments, the cell is a fibrocyte, fibroblast, keratinocyte or endothelial cell. In yet other embodiments, the cell is in vivo or ex vivo. The agent can comprise an inhibitor of a miRNA located in the miR-17-2 cluster. In some embodiments, the miRNA located in the miR-17-2 cluster is miR-19. In some embodiments, the miRNA located in the miR-17-2 cluster is both miR-19 and miR-92. In some embodiments, the agent comprises an inhibitor of miR-19 (e.g., miR-19 inhibitor selected from Table 1) alone or in combination with an inhibitor of miR-92 (e.g., miR-92 inhibitor selected from Table 2). In embodiments utilizing a inhibitor of miR-92, the method can further comprise measuring the expression of one or more targets of miR-92 and comparing the expression or activity of the one or more genes that are targets of miR-92 to a pre-determined reference level or level of the one or more genes that are targets of miR-92 in a control sample, wherein the comparison is indicative of the agents' ability to promote angiogenesis. The one or more targets of miR-92 can be one or more of the targets disclosed in US20160208258, the contents of which are hereby incorporated by reference in their entirety.

Measuring or detecting the expression of a gene can be performed in any manner known to one skilled in the art and such techniques for measuring or detecting the level of a gene are well known and can be readily employed. Gene expression levels may be determined measuring the mRNA levels of a gene or the protein levels of a protein that the gene encodes. A variety of methods for detecting gene expression have been described and include Western blotting, enzyme linked immunoassay (ELISA), immunocytochemistry, immunohistochemistry, Northern blotting, microarrays, electrochemical methods, bioluminescent, bioluminescent protein reassembly, BRET-based (BRET: bioluminescence resonance energy transfer), RT-PCR, fluorescence correlation spectroscopy and surface-enhanced Raman spectroscopy. Commercially available kits can also be used. The methods for detecting gene expression can include hybridization-based technology platforms and massively-parallel next generation sequencing that allow for detection of multiple gene simultaneously.

In some embodiments, a method for determining the therapeutic efficacy of a therapeutic for treating a condition (e.g., peripheral artery disease or a wound) in a subject comprises selecting a subject for treatment with a therapeutic (e.g., a miR-19 inhibitor alone or in combination with a miR-92 inhibitor), selecting a subject for treatment with a therapeutic (e.g., a miR-19 inhibitor alone or in combination with a miR-92 inhibitor), or evaluating an agent's ability to promote angiogenesis or wound healing; the level of expression and/or activity of one or more genes that are targets of miR-19 (e.g, miR-19a or miR-19b) such as FZD4 or LRP6, is determined.

The gene expression or activity in a sample (e.g. a sample from a subject being administered the therapeutic or a sample from a subject or cell culture, in which the sample is treated with the therapeutic), can be compared to a standard amount or activity of the gene present in a sample from a subject with the condition or in the healthy population, each of which may be referred to as a reference level. In other embodiments, the level of gene expression or activity is compared to level in a control sample (a sample not from a subject with the condition) or compared to the gene expression level or activity in a sample without treatment, (e.g. taken from a subject prior to treatment with a therapeutic or a sample taken from an untreated subject, or a cell culture sample that has not been treated with the therapeutic). Standard levels for a gene can be determined by determining the gene expression level in a sufficiently large number of samples obtained from normal, healthy control subjects to obtain a pre-determined reference or threshold value. As used herein, “reference value” refers to a pre-determined value of the gene expression level or activity ascertained from a known sample.

A standard level of expression or activity can also be determined by determining the gene expression level or activity in a sample prior to treatment with the therapeutic. Further, standard level information and methods for determining standard levels can be obtained from publically available databases, as well as other sources. In some embodiments, a known quantity of another gene that is not normally present in the sample is added to the sample (i.e. the sample is spiked with a known quantity of exogenous mRNA or protein) and the level of one or more genes of interest is calculated based on the known quantity of the spiked mRNA or protein. The comparison of the measured levels of the one or more genes to a reference amount or the level of one or more of the genes in a control sample can be done by any method known to a skilled artisan.

According to the present invention, in some embodiments, a difference (increase or decrease) in the measured level of expression or activity of the gene relative to the level of the gene in the control sample (e.g., sample in patient prior to treatment or an untreated patient) or a predetermined reference value is indicative of the therapeutic efficacy of the therapeutic, a subject's selection for treatment with the therapeutic, or an agent's ability to promote or inhibit angiogenesis.

Sampling methods are well known by those skilled in the art and any applicable techniques for obtaining biological samples of any type are contemplated and can be employed with the methods of the present invention. (See, e.g., Clinical Proteolytics: Methods and Protocols, Vol. 428 in Methods in Molecular Biology, Ed. Antonia Vlahou (2008),) Samples can include any biological sample from which mRNA or protein can be isolated. Such samples can include serum, blood, plasma, whole blood and derivatives thereof, cardiac tissue, muscle, skin, hair, hair follicles, saliva, oral mucous, vaginal mucous, sweat, tears, epithelial tissues, urine, semen, seminal fluid, seminal plasma, prostatic fluid, pre-ejaculatory fluid (Cowper's fluid), excreta, biopsy, ascites, cerebrospinal fluid, lymph, cardiac tissue, as well as other tissue extract samples or biopsies, in some embodiments, the biological sample is plasma or serum.

The biological sample for use in the disclosed methods can be obtained from the subject at any point following the start of the administration of the therapeutic. In some embodiments, the sample is obtained at least 1, 2, 3, or 6 months following the start of the therapeutic intervention. In some embodiments, the sample is obtained least 1, 2, 3, 4, 6 or 8 weeks following the start of the therapeutic intervention. In some embodiments, the sample is obtained at least 1, 2, 3, 4, 5, 6, or 7 days following the start of the therapeutic intervention. In some embodiments, the sample is obtained at least 1 hour, 6 hours, 12 hours, 18 hours or 24 hours after the start of the therapeutic intervention. In other embodiments, the sample is obtained at least one week following the start of the therapeutic intervention.

The methods of the present invention can also include methods for altering the treatment regimen of a therapeutic. Altering the treatment regimen can include but is not limited to changing and/or modifying the type of therapeutic intervention, the dosage at which the therapeutic intervention is administered, the frequency of administration of the therapeutic intervention, the route of administration of the therapeutic intervention, as well as any other parameters that would be well known by a physician to change and/or modify.

In some embodiments, the treatment efficacy can be used to determine whether to continue a therapeutic intervention. In some embodiments the treatment efficacy can be used to determine whether to discontinue a therapeutic intervention. In some embodiments the treatment efficacy can be used to determine whether to modify a therapeutic intervention. In some embodiments the treatment efficacy can be used to determine whether to increase or decrease the dosage of a therapeutic intervention. In some embodiments the treatment efficacy can be used to determine whether to change the dosing frequency of a therapeutic intervention. In some embodiments, the treatment efficacy can be used to determine whether to change the number or the frequency of administration of the therapeutic intervention. In some embodiments, the treatment efficacy can be used to determine whether to change the number of doses per day, per week, times per day. In some embodiments the treatment efficacy can be used to determine whether to change the dosage amount.

This invention is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the invention.

All patent and non-patent documents referenced throughout this disclosure are incorporated by reference herein in their entirety for all purposes.

### EXAMPLES

### Example 1. MiR-19 Antagonism Promotes Collateral Dependent Blood Flow and Ischemic Recovery

The miR 17-92 cluster is important for arteriogenesis and angiogenesis, and miR-19 is a critical regulator. C57/B16J mice (6 months old) were injected subcutaneously with LNA-modified anitmiR-19 or a control antimiR at a dose of 12.5 mg/kg for 3 days prior to surgery then weekly thereafter. The antimiR-19 belongs to a class of oligonucleotides with classical LNA-containing oligonucleotide pharmacokinetic profiles as described in Elmen J. et al. (2008) “Antagonism of microRNA-122 in mice by systemically administered LNA-antimiR leads to up-regulation of a large set of predicted target mRNAs in liver” Nucleic acids research 36(4):1153-1162 and Montgomery et al., (2011) “Therapeutic inhibition of miR-208a improves cardiac function and survival during heart failure” Circulation 124(14):1537-1547, both of which are hereby incorporated by reference in their entireties. The mice were injected for 3 consecutive days and then subjected to hind limb ischemia, followed by a weekly maintenance injection throughout the experiment. In brief, following subcutaneous administration, plasma concentrations for these antimiRs typically achieve peak concentrations between 30 minutes and 1 hour after administration. Plasma clearance is biphasic with a short, initial distribution phase, followed by a longer elimination phase. Oligonucleotide accumulation is highest in the kidney and liver, with significant accumulation also observed in spleen, bone marrow and distal skin (away from the injection site). Terminal elimination half-lives are several weeks, ranging from roughly three to six weeks.

6 month-old male mice were used for all experiments since they have less capacity to completely recover post HLI. HLI was performed as described in Yu, J, et al., (2005) “Endothelial nitric oxide synthase is critical for ischemic remodeling, mural cell recruitment, and blood flow reserve” PNAS 102(31): 10999-11004 and Ackah E, et al., (2005) “Akt1/protein kinase Balpha is critical for ischemic and VEGF-mediated angiogenesis” J Clin Invest 115(8):2119-2127, both of which are hereby incorporated by reference in their entireties.

Perfusion was quantified by measuring gastrochnemius flow pre- and post-surgery, followed by weekly measurements using a deep penetrating laser doppler probe as described in J, et al., (2005) “Endothelial nitric oxide synthase is critical for ischemic remodeling, mural cell recruitment, and blood flow reserve” PNAS 102(31): 10999-11004.

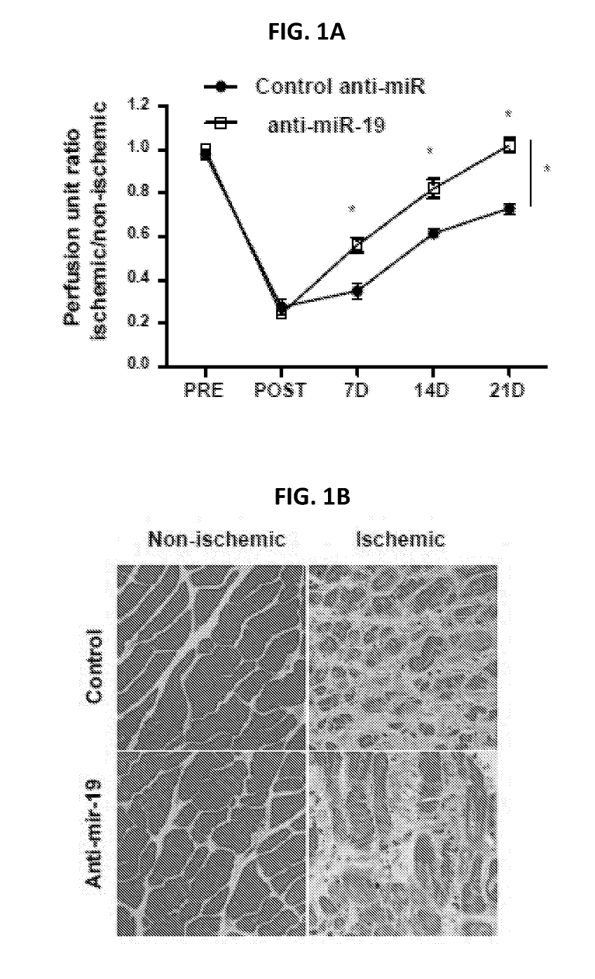

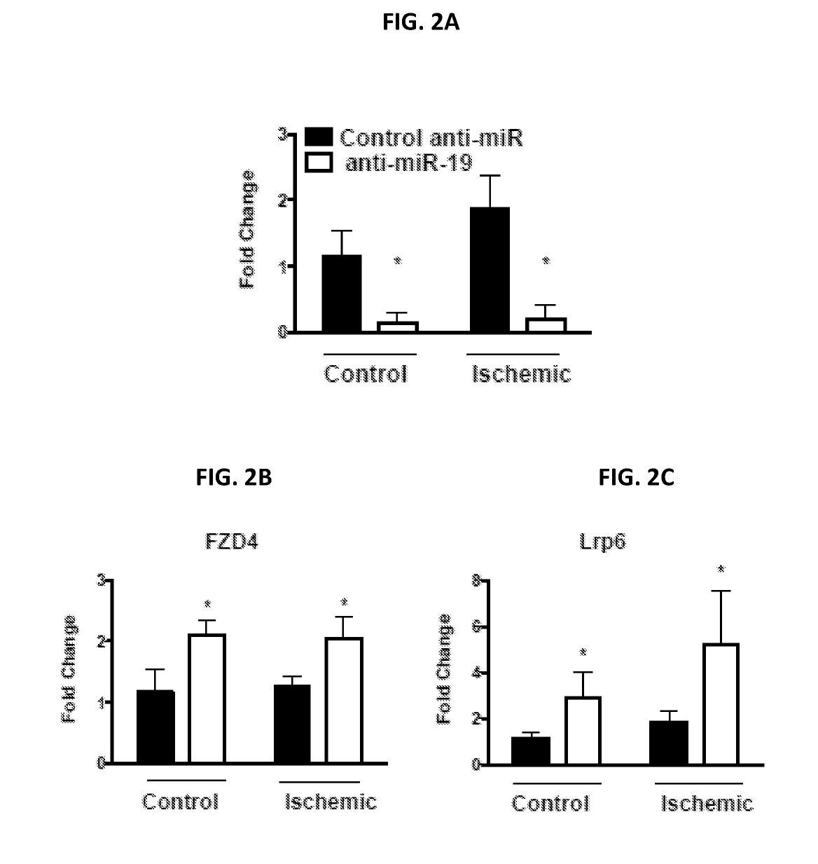

For miRNA detection and analysis of target mRNA (e.g., FZD4 and LRP6) shown in FIG. 1A and FIGS. 2A-C, total RNA from thigh muscle tissue was extracted from the mice injected with antimiR-19 or control as described herein using commercially available RNA extraction kits (e.g., miRNeasy Mini Kit (Qiagen)).

For miRNA detection (as shown in FIG. 1A), the extracted RNA was retro-transcribed using the RT² miRNA First strand kit (Qiagen) and qPCR was performed using the SYBR Green Fluor qPCR Mastermix (Qiagen). Snord66 was used as an internal normalization control. For the detection of Pri miRNA, RNA was reverse transcribed using the High Capacity RNA to cDNA kit (AB Applied Biosystems), and Real Time PCR was done using the Taqman Expression Master Mix (Applied Biosystems). GAPDH was used as an internal normalization control. RNA was extracted from 3-5 biological replicates. Real-time PCR amplification reaction was performed on the iQ5 BioRad, the comparative CT method (Delta Delta Method) was used to analyze the data.

For analysis of target mRNA (e.g., FZD4 and LRP6) in FIG. 2A-C, cDNA was synthesized using iScript Synthesis (BioRad) followed by quantitative analysis using iQ SYBR Green Supermix (BioRad). Sequences of primers include: mGAPDH F′ 5′-AATGTGTCCGTCGTGGATCTGA (SEQ ID NO: 182), mGAPDH R′ 5-AGTGTAGCCCAAGATGCCCTTC (SEQ ID NO: 183), mFZD4 F′ 5′-AGAGAGAAGAGGGGGAATGG (SEQ ID NO: 184) mFZD4 R′ 5′-TGTGTGTGGGCTGAAGTGTT (SEQ ID NO: 185). mLrp6 F′ 5′-TGTGGTAAACCCCGAGAAAG (SEQ ID NO: 186) R′ 5-ATCCTGTTGGCACCTGAGA (SEQ ID NO: 187). GAPDH was used as an internal normalization control.

Additionally, the physiological role of miR-19 in vivo was assessed using an LNA-antimiR approach and HLI in BAT gal mice. Initially, aged BAT gal mice were subjected to HLI as described above and treated with subcutaneous injections (3 days before and 2 days after surgery) of a LNA-antimiR-19. Subsequently, the expression of b-galactosidase was examined in tissue. As seen in FIG. 1B, antimiR-19, but not control, increased reporter gene expression in capillary EC surrounding regenerating muscle fibers in ischemic tissue.

The administration of antimiR-19 improved blood flow recovery in a hindlimb ischemia mouse model compared to control antimiR, which is a model for peripheral artery disease, vascular remodeling and ischemia (FIG. 1A). Treatment with antimiR-19 reduced miR-19 levels in the tissue of the mice (FIG. 2A) and upregulated FRZD4 and LRP6, directs targets for miR-19 that were identified (FIGS. 2B and 2C). More specifically, quantitative reverse transcriptase polymerase chain reaction (qRT-PCR) analysis of thigh muscle tissue confirmed miR-19 repression in animals treated with the LNA modified antimiR-19 (FIG. 1). Sample analysis depicted an overall significant increase in FZD4 and LRP6 mRNA levels in mice treated with LNA miR-19 expression as compared to control LNA-miR treated mice (FIGS. 2B-C). Thus, antimiR-19 may be useful therapy for peripheral arterial disease or myocardial ischemia, where collateral blood supply can be a key determinant of muscle function.

### Example 2. MiR-19 Directly Targets FZD4 and LRP6

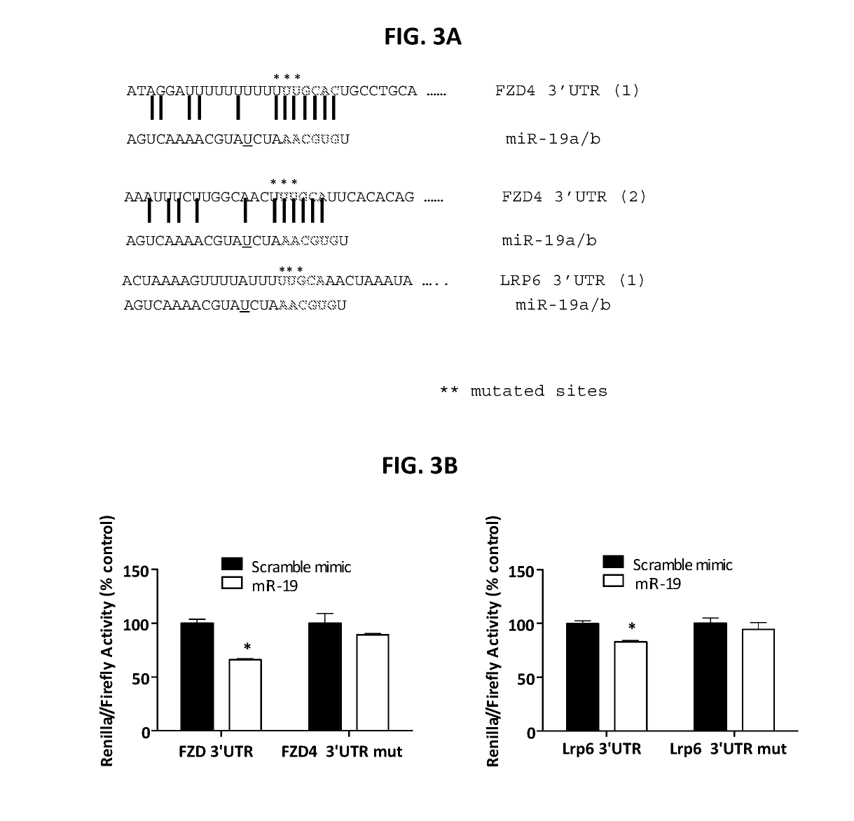

In this example, based on the presence of putative miR-19 predicted binding sites in the 3′ UTR of FZD4 and LRP6 (FIG. 3A), the question of whether miR-19 directly targeted FZD4 and LRP6 was examined. HEK293T cells were transfected with the full length 3′UTR of FZD4 or LRP6 cloned into a bicistronic renilla/firefly luciferase reporter vector and co-transfected with a miR-19 mimic or a negative control mimic and a luciferase reporter assay was performed. Briefly, a 1 kb fragment of the mouse FZD4 3′UTR or a 400 bp fragment of the mouse LRP6 3′UTR were generated by PCR and cloned into the psi-CHECK-2 vector (Promega). HEK293 cells were plated into 24-well plates at 9×10⁴ cells/well 24 hrs before transfection. 50 ng of psi-CHECK-2 reporter plasmid containing the cloned 3′UTR or its mutant and 60 nM miRNA mimic (miR-19; Thermo Scientific Dharmacon) were co transfected using lipofectamine 2000 (Invitrogen) and oligofectamine (Invitrogen), respectively, in triplicate wells. Luciferase assays were performed 48 hrs after transfection using the Dual Luciferase Reporter Assay System (Promega).

As shown in FIG. 3B, miR-19 mimics reduced the levels of the FZD4 and LRP6 3′UTR reporter demonstrating that both these receptors are a targets of miR-19. Mutagenesis of the seed sequences of the predicted miR-19 binding sites as shown in FIG. 3A restored luciferase expression, thus confirming specificity of the interaction between miR-19 and the FZD4 and LRP6 3′UTR. Since miR-19 reduced luciferase activity of the 3′UTR construct in a sequence specific manner, but did not directly reduce LRP6 mRNA levels, it may be likely that miR-19 regulates the translational efficiency of LRP6.

### Example 3. MiR-19 Effects WNT/β Catenin Mediated Gene Expression in Endothelial Cells (EC)

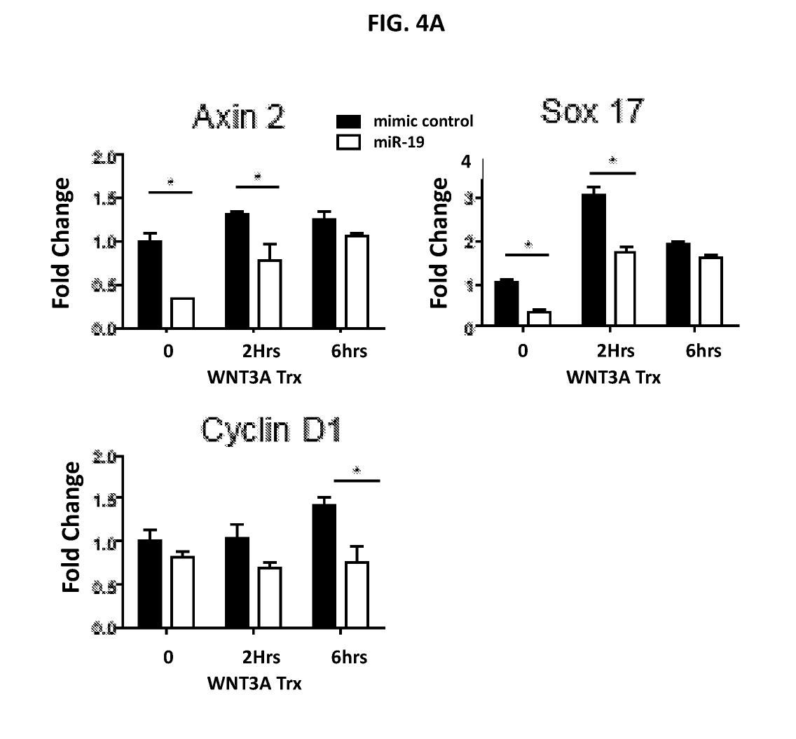

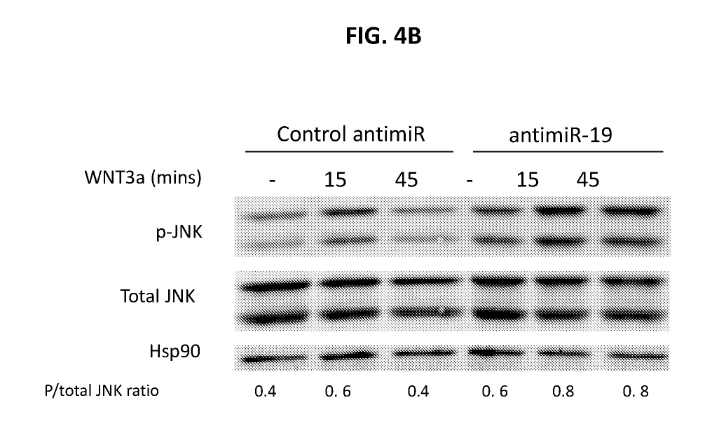

Mouse lung endothelial cells (MLECs) were isolated from mice as described in Lanahan A A, et al. (2010) “VEGF reporter 2 endocytic trafficking regulates arterial morphogenesis” Dev Cell 18(5):713-724, which is hereby incorporated by reference in its entirety. Briefly, lungs were excised from euthanized mice, pulled from 5 mice, minced and digested in freshly prepared 2 mg/ml collagenase in PBS for 45 min at 4° C. The homogenized digest was passed multiple times through a 14-gauge needle and filtered through a 70 μm cell strainer. Cell homogenates were incubated with Dynabeads (Dynal USA) conjugated with anti-mouse PECAM-1 antibody (Pharmingen) followed by cell sorting using a magnetic cell separator. Cells were plated on 0.1% gelatin-coated dishes. When cells reached 70% confluency, a second immune-selection was performed and cells were plated and referred to as passage 0. Cells were propagated in 20% FBS, supplemented with MEM non-essential amino acids (Gibco), gentamicin and amphotericin B, penicillin streptomycin, L-glutamine, endothelial mitogen (Biomed Tech Inc.) and heparin 100 μg/ml (Sigma) in DMEM (Lonza 12-709F). Subsequent in vitro experiments (i.e., FIGS. 4A and 4B) involving endothelial cells (EC) were done using primary EC cultures using cells between passage 0-3.

In FIG. 4A, the mRNA expression of WNT3a transcriptionally regulated genes were analyzed via qRT-PCR. MLECs were transfected with either miR-19 mimic (30 nM; Thermo Scientific Dharmacon) or mimic control (30 nM). 48 hours post-transfection, the MLECs were serum starved in 0.5% fetal bovine serum (FBS) for 3 hours and stimulated with control conditioned media (CM) or 10% WNT3a CM for 2-6 hours. WNT3a conditioned media and control conditioned media was prepared and tested using L WNT3a cells (ATCC CRL-2647) and control L cells (ATCC CRL-2648) as previously described in Wilbert J, et al., (2002) “A transcriptional response to Wnt protein in human embryonic carcinoma cells” BMC Dev Biol 2:8, which is hereby incorporated by reference in its entirety.

As can be seen in FIG. 4A, miR-19 transfected cells resulted in reduced expression of several β-catenin dependent genes in response to WNT3a treatment-including Axin2, Sox17 and Cyclin D1.

In addition, since FZD4 is a component of both the canonical (β-catenin) and non-canonical (planar cell polarity, PCP) pathways, FZD4 coupling to c-Jun NH2-terminal kinase (JNK) was examined. MLECs were plated as described above and subsequently transfected with control or anti-miR-19 (60 nM each) for 48 hours prior to WNT3a stimulation as described above. MLECs were starved for 4 hours then treated with WNT3a conditioned media for 0, 15, or 45 minutes. The conditioned media was prepared as described above. Lysates were prepared as previously described in the art, collected, and run on SDS-PAGE gel and immunoblotted for p-JNK, total JNK, and HSP90. Antibodies used included HSP90 (BD 610419).

As shown in FIG. 4B, treatment of MLECs with antimiR-19 enhanced WNT3a stimulation of p-JNK, implying that miR-19 can also negatively regulate PCP signaling.

Collectively, these data show that miR-19, negatively regulates the WNT signaling, and in turn, regulates aspects of arterial development.

### Example 4: miR-92 Antagonism Promotes Wound Healing in a Diabetic Wound Model and Combination miR-92 and miR-19 Antagonism is Better than miR-92 Antagonism Alone

miR-92 (SEQ ID NO. 22) and miR-19 (SEQ ID NO: 11) antagonists were tested in an in vivo chronic wound model for acceleration of wound healing. Db/db (BKS.Cg Dock(Hom) 7m+/+Leprdb/j) mice develop type II diabetes and wound healing impairments by 6 weeks of age. Age and sex matched adult mice were anesthetized and the dorsum was depilated. Two 6 mm diameter excisional punch wounds were made on their backs equidistant between shoulders and hips, on either side of the spine, and both wounds were covered with a semi-occlusive dressing.

Compounds were applied via intradermal injection at multiple sites around the wound margin at the time of surgery, as well as on post-operative days 2, 4 and 8. Mice administered a vehicle control were used as negative controls.

Animals were sacrificed at day 10 post-surgery. Histology analysis was performed in order to assess the percentage of re-epithelialization, the percentage of granulation tissue ingrowth, and the thickness and cross-sectional area of neo-epithelium and granulation tissue. Histology analysis was performed by fixing one half of each skin wound in 10% neutral buffered formalin for 24 hours and embedding in paraffin according to standard protocols. 4 um tissue sections were deparaffinized and stained with hematoxylin and eosin. Full slide scans were performed at 20× magnification using an Aperio AT2 scanner and images were analyzed for % re-epithelialization, % granulation tissue ingrowth, as well as thickness and cross-sectional area of neo-epithelium and granulation tissue using Aperio ImageScope.

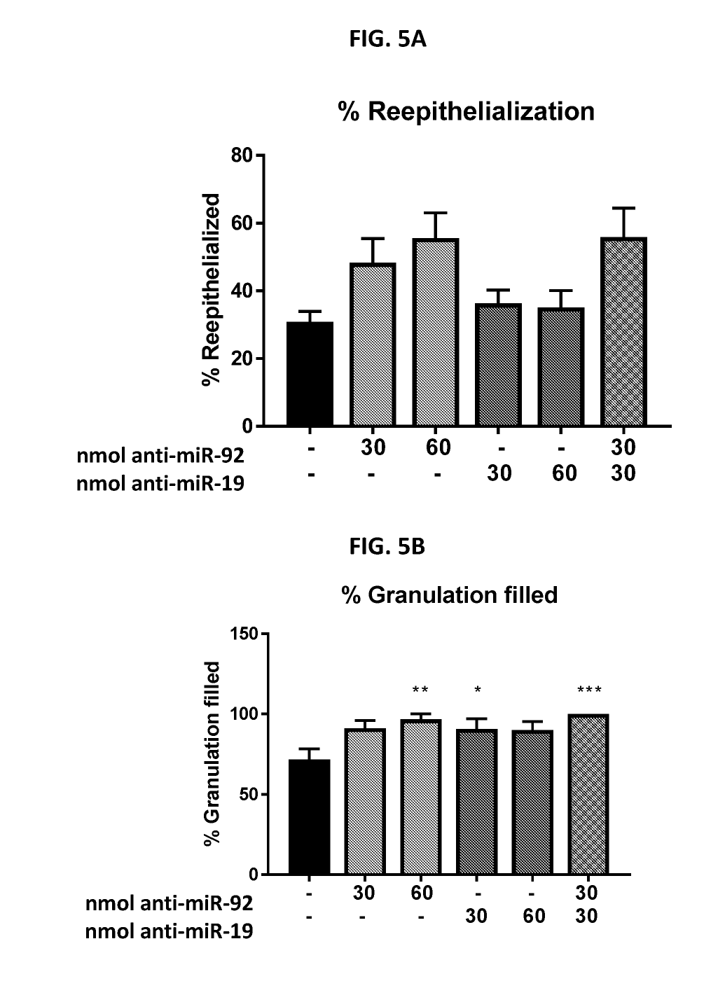

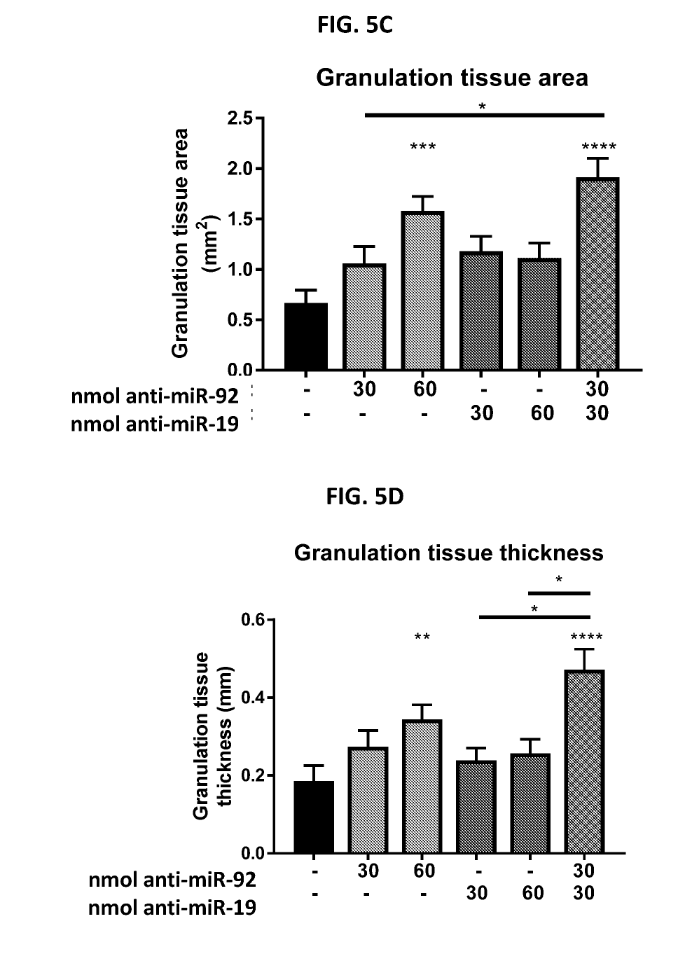

Data from this study are presented in FIG. 5A-D. Data in FIG. 5A illustrated that antimiR-92 as well as antimiR-92 plus antimiR-19 increased wound re-epithelialization as compared to control wounds. The magnitude of change of the combination antimiR-92 and antimiR-19 treatment was equivalent to the high dose (60 nmol) of antimiR-92 alone. Data in FIG. 5B illustrated that both antimiR-92 and antimiR-19 increased the ingrowth of granulation tissue into a skin wound. Data in FIG. 5C and FIG. 5D illustrate that antimiR-92 increased the granulation tissue area and thickness, respectively, in a dose-dependent fashion, that antimiR-19 alone had an effect on granulation tissue area and thickness, and that the combination antimiR-92 and antimiR-19 treatment had a greater effect than the high dose (60 nmol) of antimiR-92.

This study demonstrated that a miR-92 antagonist increased wound healing in a dose-dependent fashion, as measured by an increase in re-epithelialization, granulation tissue ingrowth, granulation tissue area and granulation tissue thickness. These results are consistent with the results presented in US20160208258, the contents of which are hereby incorporated by reference in their entirety. Treatment with a miR-19 antagonist showed improvements in all parameters as compared to control wounds, including granulation tissue ingrowth at the 30 nmol dose. Compared to either oligonucleotide alone, the combination of 30 nmol miR-92 inhibitor and 30 nmol miR-19 inhibitor showed substantial improvements in wound healing. These results illustrate a combinatorial effect of miR-92 and miR-19 antagonism on improving wound healing.

In all examples, where applicable, statistical analysis was performed with Prism 5 Software. Significance was tested by two-tailed unpaired Student's t-test or two-way ANOVA with Bonferroni correction for multiple comparisons when appropriate. All values are expressed as means±SEM.

All publications, patents, and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

1. A method for promoting wound healing in a subject in need thereof, comprising administering an oligonucleotide inhibitor of miR-19 comprising a sequence complementary to miR-19.

2. The method of claim 1, wherein the oligonucleotide inhibitor of miR-19 reduces function or activity of miR-19.

3. The method of claim 1, wherein the oligonucleotide inhibitor of miR-19 is selected from Table 1.

4. The method of any one of claims 1-3, further comprising administering an additional agent for promoting wound healing.

5. The method of claim 4, wherein the additional agent is an oligonucleotide inhibitor of miR-92 comprising a sequence complementary to miR-92.

6. The method of claim 5, wherein the administration of the oligonucleotide inhibitor of miR-92 reduces function or activity of miR-92.

7. The method of claim 5 or 6, wherein the oligonucleotide inhibitor of miR-92 is selected from Table 2.

8. The method of any one of claims 4-7, wherein the oligonucleotide inhibitor of miR-19 and the additional agent are administered sequentially.

9. The method of any one of claims 4-7, wherein the oligonucleotide inhibitor of miR-19 and the additional agent are administered simultaneously.

10. The method of claim 1-9, further comprising adding a growth factor.

11. The method of claim 10, wherein the growth factor is platelet derived growth factor (PDGF) and/or vascular endothelial growth factor (VEGF).

12. The method of any one of claims 1-11, wherein the subject is human.

13. The method of any one of claims 1-12, wherein the subject suffers from diabetes.

14. The method of any one of claims 1-13, wherein the wound healing is for a chronic wound, diabetic foot ulcer, venous stasis leg ulcer or pressure sore.

15. The method of any one of claims 1-3, wherein the administration of the oligonucleotide inhibitor of miR-19 produces an increased rate of re-epithelialization, granulation, and/or neoangiogenesis during wound healing as compared to no treatment.

16. The method of any one of claims 5-9, wherein the administration of the oligonucleotide inhibitor of miR-19 and the oligonucleotide inhibitor of miR-92 produces an increased rate of re-epithelialization, granulation, and/or neoangiogenesis during wound healing as compared to no treatment or treatment with either the oligonucleotide inhibitor of miR-19 or the oligonucleotide inhibitor of miR-92 alone.

17. An oligonucleotide inhibitor comprising a sequence complementary to miR-19, wherein the sequence further comprises one or more locked nucleic acid (LNA) nucleotides and one or more non-locked nucleotides, wherein at least one of the non-locked nucleotides comprises a chemical modification.

18. The oligonucleotide inhibitor of claim 17, wherein the oligonucleotide inhibitor is complementary to miR-19a.

19. The oligonucleotide inhibitor of claim 17, wherein the oligonucleotide inhibitor is complementary to miR-19b.

20. The oligonucleotide inhibitor of any one of claims 17-19, wherein the locked nucleic acid (LNA) nucleotides has a 2′ to 4′ methylene bridge.

21. The oligonucleotide inhibitor of any one of claims 17-20, wherein the chemical modification is a 2′ O-alkyl or 2′ halo modification.

22. The oligonucleotide inhibitor of any one of claims 17-21, wherein the oligonucleotide inhibitor has a 5′ cap structure, 3′ cap structure, or 5′ and 3′ cap structure.

23. The oligonucleotide inhibitor of any one of claims 17-22, further comprising a pendent lipophilic group.

24. The oligonucleotide inhibitor of claim 17, wherein the sequence is selected from Table 1.

25. A pharmaceutical composition comprising the oligonucleotide inhibitor of any one of claims 17-24, or a pharmaceutically-acceptable salt thereof, and a pharmaceutically-acceptable carrier or diluent.

26. The pharmaceutical composition of claim 25, further comprising an oligonucleotide inhibitor of miR-92 comprising a sequence complementary to miR-92.

27. The pharmaceutical composition of claim 26, wherein the sequence is selected from Table 2.

28. The pharmaceutical composition of claim 26 or 27, wherein a molar ratio of an amount of the oligonucleotide inhibitor of miR-19 to an amount of the oligonucleotide inhibitor of miR-92 in the composition is from about 1:99 to about 99:1.

29. The pharmaceutical composition of claim 28, wherein the molar ratio of the oligonucleotide inhibitor of miR-19 to the oligonucleotide inhibitor of miR-92 is about 1:1.

30. A method of treating a wound in a subject in need thereof, comprising administering to the subject the pharmaceutical composition of any one of claims 25-29.

31. The method of claim 30, wherein the wound is a chronic wound, diabetic foot ulcer, venous stasis leg ulcer or pressure sore.

32. A method for evaluating or monitoring the efficacy of a therapeutic for modulating wound healing in a subject receiving the therapeutic comprising:
a) measuring the expression of one or more genes that are targets of miR-19 from a sample from a subject; and
b) comparing the expression of the one or more genes that are targets of miR-19 to a pre-determined reference level or level of the one or more genes that are targets of miR-19 in a control sample, wherein the comparison is indicative of the efficacy of the therapeutic, wherein the therapeutic is an oligonucleotide comprising a sequence selected from Table 1.

33. The method of claim 32, wherein the one or more genes that are targets of miR-19 is frizzled-4 (FZD4) or low-density lipoprotein receptor-related protein 6 (LRP6).

34. The method of claim 32 or 33, wherein the therapeutic modulates miR-19 function and/or activity.

35. The method of any one of claims 32-34, wherein the subject suffers from ischemia, myocardial infarction, chronic ischemic heart disease, peripheral or coronary artery occlusion, ischemic infarction, stroke, atherosclerosis, acute coronary syndrome, coronary artery disease, carotid artery disease, diabetes, chronic wound(s), peripheral vascular disease or peripheral artery disease.

36. The method of any one of claims 32-35, wherein the subject is a human.

37. A method for evaluating an agent's ability to promote angiogenesis or wound healing comprising:
a) contacting a cell with the agent, wherein the agent is an oligonucleotide inhibitor comprising a sequence selected from Table 1;
b) measuring the expression of one or more genes that are targets of miR-19 in the cell contacted with the agent; and
c) comparing the expression of the one or more genes that are targets of miR-19 to a pre-determined reference level or level of the one or more genes that are targets of miR-19 in a control sample, wherein the comparison is indicative of the agent's ability to promote angiogenesis or wound healing.

38. The method of claim 37, wherein the one or more genes that are targets of miR-19 is FZD4 or LRP6.

39. The method of claim 37 or 38, further comprising determining miR-19 function and/or activity in the cell contacted with the agent.

40. The method of any of claims 37-39, wherein the cell is a mammalian cell.

41. The method of claim 40, wherein the cell is a cardiac cell, muscle cell, fibrocyte, fibroblast, keratinocyte or endothelial cell.

42. The method of any one of claims 37-41, wherein the cell is in vitro, in vivo or ex vivo.
